(19) United States
(12) Patent Application Publication
(10) Pub. No.: US 2020/0069592 A1
(43) Pub. Date: Mar. 5, 2020

(54) HSP90 INHIBITOR ORAL FORMULATIONS AND RELATED METHODS

(71) Applicant: Samus Therapeutics, Inc., Topsfield, MA (US)

(72) Inventor: John Amedio, Franklin, MA (US)

(73) Assignee: Samus Therapeutics, Inc., Topsfield, MA (US)

(21) Appl. No.: 16/607,610

(22) PCT Filed: Apr. 24, 2018
(86) PCT No.: PCT/US2018/029157

§ 371 Date: Oct. 23, 2019

Related U.S. Application Data
(60) Provisional application No. 62/627,229, filed on Feb. 7, 2018.
(60) Provisional application No. 62/627,237, filed on Feb. 7, 2018.
(60) Provisional application No. 62/588,893, filed on Nov. 20, 2017.
(60) Provisional application No. 62/588,897, filed on Nov. 20, 2017.
(60) Provisional application No. 62/532,985, filed on Jul. 14, 2017.
(60) Provisional application No. 62/532,987, filed on Jul. 14, 2017.
(60) Provisional application No. 62/489,438, filed on Apr. 24, 2017.
(60) Provisional application No. 62/489,434, filed on Apr. 24, 2017.

(51) Int. Cl. A61K 9/20; A61K 47/10; A61K 31/52; A61K 47/14; A61K 9/00; A61K 9/48

(52) U.S. Cl. CPC A61K 9/2054; A61K 47/10; A61K 31/52; A61K 47/14; A61K 9/4891; A61K 9/0053; A61K 9/2027; A61K 9/4858; A61K 9/4866; A61K 9/2018

(57) ABSTRACT

Provided herein are novel and improved oral formulations for Hsp90 inhibitors.

### RELATED APPLICATIONS

This application claims the benefit under 35 U.S.C. § 119 of U.S. Provisional Application Ser. No. 62/489,438, filed Apr. 24, 2017, U.S. Provisional Application Ser. No. 62/489,434, filed Apr. 24, 2017; U.S. Provisional Application Ser. No. 62/532,985, filed Jul. 14, 2017, U.S. Provisional Application Ser. No. 62/532,987, filed Jul. 14, 2017, U.S. Provisional Application Ser. No. 62/588,893, filed Nov. 20, 2017, U.S. Provisional Application Ser. No. 62/588,897, filed Nov. 20, 2017, U.S. Provisional Application Ser. No. 62/627,229, filed Feb. 7, 2018, and U.S. Provisional Application Ser. No. 62/627,237, filed Feb. 7, 2018, the entire contents of which are incorporated herein by reference.

### BACKGROUND

The Hsp90 family of proteins has four recognized members in mammalian cells: Hsp90-alpha (α) and -beta (β), GRP94 and TRAP-1. Hsp90-alpha and -beta exist in the cytosol and the nucleus in association with many other proteins. The Hsp90 family collectively represents the most abundant cellular chaperones, and it has been proposed to function in several beneficial ways including for example as part of the cellular defense against stress such as exposure heat or other environmental stress. However, it has also been postulated to facilitate the stability and function of mutated proteins such as for example mutated p53. Hsp90 has also been found to work collectively with other heat shock proteins to form an epichaperome. Based on these various functions, Hsp90 and, in some instances, downstream effectors of Hsp90 such as the epichaperome have been identified as viable therapeutic targets for therapeutic agents.

### SUMMARY

This disclosure is premised, in part, on the unexpected finding that certain oral formulations for inhibitors of Hsp90, Hsp90 isoforms and Hsp90 homologs can be administered orally with therapeutic efficacy on par with formulations administered via other routes. Certain oral administration of this inhibitor class can improve the absorption of these agents, thereby increasing their bioavailability and ultimately their therapeutic efficacy. Oral administration may also result in greater patient compliance and/or decreased toxicity, thereby contributing to better outcomes as well.

Provided in one aspect is a minitablet comprising an Hsp90 inhibitor, a binder/diluent, optionally microcrystalline cellulose, a disintegrant, optionally crospovidone, an anti-tack agent/flow aid, optionally colloidal silicon dioxide, and a lubricant, optionally magnesium stearate. The minitablet may be a delayed release minitablet and may further comprise a delayed release coating comprising a delayed release polymer, optionally methacrylic acid copolymer, a plasticizer, optionally triethyl citrate, and anti-tack agent/flow aids, optionally colloidal silicon dioxide and/or talc.

Provided in one aspect is a delayed release capsule (or delayed release capsular formulation) comprising a minitablet comprising an Hsp90 inhibitor, a binder/diluent, optionally microcrystalline cellulose, a disintegrant, optionally crospovidone, an anti-tack agent/flow aid, optionally colloidal silicon dioxide, and a lubricant, optionally magnesium stearate; and a delayed release coating comprising a delayed release polymer, optionally methacrylic acid copolymer, a plasticizer, optionally triethyl citrate, anti-tack agent/flow aids, optionally colloidal silicon dioxide and/or talc, and a capsule, optionally an HMPC capsule. The capsule may comprise a plurality of minitablets.

As used herein, a capsule formulation and a capsular formulation are used interchangeably.

In some embodiments, the foregoing delayed release capsules (or delayed release capsular formulations) may further comprise as a w/w percentage of the total weight of the capsule (or capsular formulation), in the minitablet, about 70-80% Hsp90 inhibitor, about 3-4% binder/diluent, optionally microcrystalline cellulose, about 4-5% disintegrant, optionally crospovidone, about 1-2% anti-tack agent/flow aid, optionally colloidal silicon dioxide, and about 0.1-2% lubricant, optionally magnesium stearate; and in the delayed release coating, about 8-9% delayed release polymer, optionally methacrylic acid copolymer, about 1-2% plasticizer, optionally triethyl citrate, and about 1-2% anti-tack agent/flow aid, optionally colloidal silicon dioxide and/or talc.

In some embodiments, the foregoing delayed release capsules (or delayed release capsular formulations) may further comprise one or more minitablets.

Provided in one aspect is a minitablet comprising an Hsp90 inhibitor, a binder/diluent, optionally microcrystalline cellulose, a disintegrant, optionally crospovidone, an anti-tack agent/flow aid, optionally colloidal silicon dioxide, and a lubricant, optionally magnesium stearate. The minitablet may be an extended release minitablet and may further comprise a delayed release coating comprising a delayed release polymer, optionally methacrylic acid copolymer, a plasticizer, optionally triethyl citrate, anti-tack agent/flow aids, optionally colloidal silicon dioxide and/or talc; and an extended release coating comprising a plasticizer, optionally triethyl citrate, anti-tack agent/flow aids, optionally colloidal silicon dioxide and/or talc, and a rate controlling polymer, optionally ammonio methacrylate copolymer.

Provided in one aspect is an extended release capsule (or extended release capsular formulation) comprising a minitablet core comprising an Hsp90 inhibitor, a binder/diluent, optionally microcrystalline cellulose, a disintegrant, optionally crospovidone, an anti-tack agent/flow aid, optionally colloidal silicon dioxide, and a lubricant, optionally magnesium stearate; a delayed release coating comprising a delayed release polymer, optionally methacrylic acid copolymer, a plasticizer, optionally triethyl citrate, anti-tack agent/flow aids, optionally colloidal silicon dioxide and/or talc; an extended release coating comprising a plasticizer, optionally triethyl citrate, anti-tack agent/flow aids, optionally colloidal silicon dioxide and/or talc, and a rate controlling polymer, optionally ammonio methacrylate copolymer, and a capsule, optionally an HMPC capsule.

In some embodiments, the foregoing delayed extended capsules (or extended release capsular formulations) may further comprise as a w/w percentage of the total weight of the capsule in the minitablet, about 70-80% Hsp90 inhibitor, about 3-4% binder/diluent, optionally microcrystalline cellulose, about 4-5% disintegrant, optionally crospovidone, about 1-2% anti-tack agent/flow aid, optionally colloidal silicon dioxide, and about 0.1-2% lubricant, optionally magnesium stearate; in the delayed release coating, about 7-10% delayed release polymer, optionally methacrylic acid copolymer, about 1-2% plasticizer, optionally triethyl citrate, about 2-4% anti-tack agent/flow aids, optionally colloidal silicon dioxide and/or talc; and in the extended release coating, about 0.5-2% plasticizer, optionally triethyl citrate, about 0.1-1.5% anti-tack agent/flow aids, optionally colloidal silicon dioxide and/or talc, and about 0.01-1% rate controlling polymer, optionally ammonio methacrylate copolymer.

In some embodiments of the foregoing delayed extended capsules (or extended release capsular formulations), the capsule may be a slow release, medium release or fast release capsule.

Provided in one aspect is a capsule (or capsular formulation) comprising an Hsp90 inhibitor, a diluent, optionally microcrystalline cellulose, a disintegrant, optionally croscarmellose sodium, a lubricant, optionally magnesium stearate, and a capsule, optionally a gelatin capsule. In some embodiments, the capsule comprises as a w/w percentage of the total weight of the capsule about 20-30% Hsp90 inhibitor, about 70-80% diluent, optionally microcrystalline cellulose, about 0.1-1% disintegrant, optionally croscarmellose sodium, about 0.1-1% lubricant, optionally magnesium stearate, and a capsule, optionally a gelatin capsule.

Provided in one aspect is a capsule (or capsular formulation) comprising an Hsp90 inhibitor, povidone or povidone derivative, methacrylic acid copolymer, amino methacrylate copolymer hypromellose acetate succinate or hypromellose, microcrystalline cellulose, croscarmellose sodium, magnesium stearate, and a capsule, optionally wherein components of the capsule are prepared using hot melt extrusion. In some embodiments, the capsule (or capsular formulation) comprises, as a w/w percentage of the total weight of the capsule (or capsular formulation), about 5-15% Hsp90 inhibitor, about 20-30% povidone, or povidone derivative, methacrylic acid copolymer, amino methacrylate copolymer hypromellose acetate succinate or hypromellose, about 50-65% microcrystalline cellulose, about 5-15% croscarmellose sodium, and about 0.5-1.5% magnesium stearate.

Provided in one aspect is a capsule (or capsular formulation) comprising an Hsp90 inhibitor, a binder, optionally Gelucire 50/13, a diluent, optionally lactose monohydrate, a disintegrant, optionally croscarmellose sodium, and a capsule, optionally wherein components of the capsule are prepared using hot melt granulation. In some embodiments, the capsule (or capsular formulation) comprises, as a w/w percentage of the total weight of the capsule (or capsular formulation), about 1-44% Hsp90 inhibitor, about 10-30% binder, optionally Gelucire 50/13, about 30-73% diluent, optionally lactose monohydrate, and about 1-10% disintegrant, optionally croscarmellose sodium.

Provided in one aspect is a capsule (or capsular formulation) comprising an Hsp90 inhibitor, and a disintegrant, optionally croscarmellose sodium.

Provided in one aspect is a capsule (or capsular formulation) comprising an Hsp90 inhibitor, and sodium starch glycolate.

Provided in one aspect is a capsule (or capsular formulation) comprising a hot melt micronized Hsp90 inhibitor, and glycerol monostearate.

Provided in one aspect is a capsule (or capsular formulation) comprising a hot melt micronized Hsp90 inhibitor, and Gelucire.

Provided in one aspect is a capsule (or capsular formulation) comprising a hot melt micronized Hsp90 inhibitor, and Vitamin E TPGS.

Provided in one aspect is a capsule (or capsular formulation) comprising a hot melt Hsp90 inhibitor, and glycerol monostearate.

Provided in one aspect is a capsule (or capsular formulation) comprising a hot melt Hsp90 inhibitor, and Gelucire.

Provided in one aspect is a capsule (or capsular formulation) comprising a hot melt Hsp90 inhibitor, and Vitamin E TPGS.

Provided in one aspect is a capsule (or capsular formulation) comprising micronized Hsp90 inhibitor.

Provided in one aspect is a capsule (or capsular formulation) comprising micronized blend of Hsp90 inhibitor.

Provided in one aspect is a spray dry dispersion tablet comprising an Hsp90 inhibitor and one or more excipients as provided in Table 10, and wherein the PVP VA can be substituted with HPMC AS or PVP K30, and wherein Compound 1 can be substituted with another Hsp90 inhibitor. For example, Compound 1 may be without limitation Compound 1a or Compound 2 or Compound 2a. In some embodiments, the ratio of PVP VA to Compound 1 (or without limitation to Compound 1a or Compound 2 or Compound 2a) can be substituted with 1:1 or 2:1.

Provided in one aspect is a tablet comprising an Hsp90 inhibitor, one or more fillers/bulking agents, optionally lactose, microcrystalline cellulose, mannitol, and/or povidone, one or more disintegrants, optionally hydroxypropyl cellulose and/or croscarmellose sodium, an eluant, optionally fumed silica, and one or more lubricants, optionally magnesium stearate and/or sodium stearyl fumarate, optionally wherein the tablet is prepared using a wet granulation-dry blend (WG-DB) method. In some embodiments, the tablet is an immediate release tablet. In some embodiments, the tablet comprises a delayed release coating.

Provided in one aspect is a capsule (or capsular formulation) comprising an Hsp90 inhibitor, cornstarch, microcrystalline cellulose, fumed silicon dioxide, polysorbate 80, gelatin, water, magnesium stearate, and a capsule, optionally wherein components of the capsule are prepared using wet granulation.

Provided in one aspect is an oral disintegrating tablet comprising an Hsp90 inhibitor, a filler or binder, optionally mannitol (e.g., Pearlitol 300DC), sucrose, silicified microcrystalline cellulose (e.g., prosolv HD90), or lactose, a disintegrant, optionally crospovidone (e.g., polyplasdone XL), L-HPC, Pharmaburst, PanExcea, or F-Melt, a lubricant, optionally Pruv or Lubripharm, and/or a glidant, optionally fumed silica, and/or a dispersion agent, optionally calcium silicate.

Provided herein are any of the foregoing minitablets, capsules (or capsular formulations) or tablets comprising an Hsp90 inhibitor having a structure of any one of Formulae I-XIV.

Provided herein are any of the foregoing minitablets, capsules (or capsular formulations) or tablets comprising an Hsp90 inhibitor that is Compound 1. Provided herein are any of the foregoing minitablets, capsules (or capsular formulations) or tablets comprising an Hsp90 inhibitor that is Compound 1a. Provided herein are any of the foregoing minitablets, capsules (or capsular formulations) or tablets comprising an Hsp90 inhibitor that is Compound 2. Provided herein are any of the foregoing minitablets, capsules (or capsular formulations) or tablets comprising an Hsp90 inhibitor that is Compound 2a.

Provided herein are any of the foregoing minitablets, capsules (or capsular formulations) or tablets comprising a dosage strength of the Hsp90 inhibitor in the range of about 0.1 mg to about 500 mg, including but not limited to more specifically a dosage strength that is at least 0.1 mg, at least 0.5 mg, at least 1 mg, at least 5 mg, at least 10 mg, at least 50 mg, or at least 100 mg of the Hsp90 inhibitor, and even more specifically a 0.1 mg, 0.5 mg, 1 mg, 5 mg, 10 mg, 50 mg, or 100 mg dosage strength of the Hsp90 inhibitor.

Provided herein are any of the foregoing minitablets, capsules (or capsular formulations) or tablets in singular form or in a plurality.

Provided herein are any of the foregoing minitablets, capsules (or capsular formulations) or tablets in a plurality in a container.

Provided herein are any of the foregoing minitablets, capsules (or capsular formulations) or tablets provided in a container with a dessicant.

Provided herein is an orally administered formulation, in solution or in suspension form, comprising an Hsp90 inhibitor in methylcellulose in water. The methylcellulose may be about 0.1% to 1%. In some embodiments, it may be about 0.5%.

Provided herein is an orally administered formulation, in solution or in suspension form, comprising an Hsp90 inhibitor in a mixture of polyanionic beta-cyclodextrin derivatives of a sodium sulfonate salt tethered to the lipophilic cavity by a butyl ether group, or sulfobutyl ether (SBE) (commerically available as Captisol®). Such polyanionic beta-cyclodextrin derivatives have the following structure:

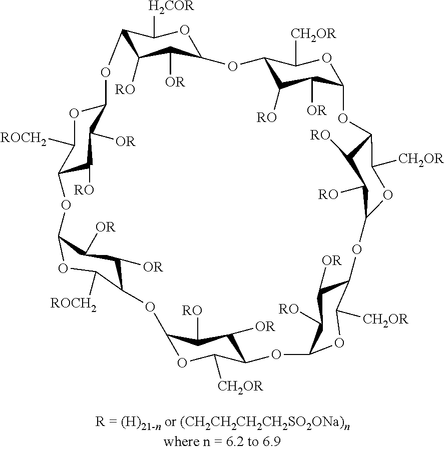

Provided herein is an orally administered formulation, in solution form or in suspension form, comprising an Hsp90 inhibitor, water, a sugar such as sucrose, glycerin, sorbitol, flavoring, buffer(s), and preservative(s). The buffer(s) may be citric acid and sodium phosphate. The preservative(s) may be methylparaben and potassium sorbate.

Provided herein is an orally administered formulation, in solution form or in suspension form, comprising an Hsp90 inhibitor, water, glycerin, sorbitol, sodium saccharin, flavouring, buffer(s), and preservative(s). The buffer(s) may be citric acid and sodium citrate. The preservative(s) may be methylparaben, potassium sorbate, and propylparaben. These may be present in the following w/w percentages: methylparaben (0.03%), potassium sorbate (0.1%), and propylparaben (0.008%). The orally administered formulation may comprise sugar(s).

Provided herein is an orally administered formulation, in solution form or in suspension form, comprising an Hsp90 inhibitor, water, a sugar such as sucrose, glycerin, sorbitol, flavoring, microcrystalline cellulose, car-boxymethylcellulose sodium, carrageenan, calcium sulfate, trisodiumn phosphate, buffer(s), anti-form agent(s) and preservative(s). The buffer(s) may be citric acid and sodium phosphate. The anti-foaming agent(s) may be dimethicone antifoam emulsion. The preservative(s) may be methylparaben and potassium sorbate.

Provided herein is an orally administered formulation, in solution form or in suspension form, comprising an Hsp90 inhibitor, water, microcrystalline cellulose, carboxymethylcellulose sodium, carrageenan, calcium sulfate, trisodium phosphate, buffer(s), anti-foaming agent(s), and preservative(s). The buffer(s) may be citric acid and sodium phosphate. The anti-foaming agent(s) may be dimethicone antifoam emulsion. The preservative(s) may be methylparaben and potassium sorbate. The orally administered formulation may comprise sugar(s).

Provided herein is an orally administered formulation, in solution form or in suspension form, comprising an Hsp90 inhibitor, water, modified food starch(es), sodium citrate, sucralose, buffer(s), anti-foaming agent(s), and preservatives(s). The buffer(s) may be citric acid, sorbic acid, and malic acid. The anti-foaming agent(s) may be simethicone. The preservative(s) may be sodium benzoate (e.g., <0.1% sodium benzoate).

In various embodiments, the orally administered formulations provided herein, including solution or suspension forms thereof, do not contain xanthan gum or other complex carbohydrate.

In various embodiments, the orally administered formulations provided herein, including solution or suspension forms thereof, do not contain sugar(s) such as sucrose, and thus are referred to herein as being “sugar-free”.

The salt to base ratio of the Hsp90 inhibitor may be about 1.14:1, and may range from about 1:5:1 to 1:1. In some embodiments, the Hsp90 inhibitor is Compound 1 in a dihydrochloride (2HCl) form. Other salt forms are contemplated including maleate, malate, oxalate and nitrate salts of the Hsp90 inhibitors provided herein including but not limited to Compound 1, Compound 1a, Compound 2, and Compound 2a.

Thus, some embodiments provide the orally administered formulation, in a solution or suspension form, comprising Compound 1 2HCl (or Compound 1a or Compound 2 or Compound 2a) in 0.5% methylcellulose in water.

In some embodiments, the Hsp90 inhibitor is provided having a mean particle size (or mean particle diameter) ranging from about 2 microns to about 12 microns. In some embodiments, the Hsp90 inhibitor is provided having a mean particle size (or mean particle diameter) ranging from about 5 microns to about 10 microns. Hsp90 inhibitor may also be provided in this mean particle size/diameter range if used for parenteral purposes (e.g., preparation of an intravenous formulation or intraperitoneal formulation, etc.). Such mean particle size/diameter ranges may be obtained by milling (including jet milling) a solid form, including a larger particulate form, of the Hsp90 inhibitor.

Also provided herein are methods for reconstituting an Hsp90 inhibitor provided in a solid or particulate form into an orally administered formulation in either a solution or suspension form. In some embodiments, the Hsp90 inhibitor is combined with a vehicle comprising water, modified food starch(es), sodium citrate, sucralose, buffer(s), anti-foaming agent(s), and preservatives(s). The buffer(s) may be citric acid, sorbic acid, and malic acid. The anti-foaming agent(s) may be simethicone. The preservative(s) may be sodium benzoate (e.g., <0.1% sodium benzoate). The Hsp90 inhibitor may be provided as a particulate form having a particle size distribution (PSD) in the range of about 2 microns to about 12 microns including about 5 microns to about 10 microns. The Hsp90 inhibitor may be prepared having this PSD using milling, such as jet milling. It may be provided separate from or together with the vehicle (e.g., the Hsp90 inhibitor and the vehicle may be provided in separate containers within the same housing, optionally with instructions on how to reconstitute the Hsp90 inhibitor using the vehicle. Reconstitution may be achieved at room temperature or at a higher temperature.

Orally administered formulations of Hsp90 inhibitors, as provided herein, may be used to treat cancer such as but not limited to breast cancer, including triple negative breast cancer, and may be administered 1, 2, 3, 4, 5, 6, or 7 times weekly or more frequently. In some embodiments, the formulation is administered 3 times weekly. Treatment may continue for 1, 2, 3, 4, 5, 6, 7, 8, 9, or 10 weeks or longer, optionally with breaks in between such time periods. For example, it may be administered for a treatment period (e.g., for 1-3 weeks of treatment, including daily treatment or treatment every other day during this period) followed by a period of no treatment (e.g., 1-3 weeks with no treatment), and this may be repeated 1, 2, 3, 4, 5, or more times. In these and other methods provided herein, the Hsp90 orally administered formulations may be solutions or suspensions, and they may include water, modified food starch(es), sodium citrate, sucralose, buffer(s), anti-foaming agent(s), and preservatives(s). The buffer(s) may be citric acid, sorbic acid, and malic acid. The anti-foaming agent(s) may be simethicone. The preservative(s) may be sodium benzoate (e.g., <0.1% sodium benzoate).

Provided herein in one aspect is a method for treating a subject having a condition characterized by abnormal Hsp90 activity, presence of mis-folded proteins, or responsiveness to Hsp90 inhibition, comprising administering one or more of any of the foregoing capsules (or capsular formulations) or tablets or orally administered formulations, in the form of solutions or suspensions, in an effective amount (e.g., a therapeutically effective amount).

In some embodiments, the condition is a cancer, optionally pancreatic or breast cancer (e.g., triple negative breast cancer), melanoma, B cell lymphoma, Hodgkin's lymphoma, or non-Hodgkin's lymphoma.

In some embodiments, the condition is a myeloproliferative neoplasm, optionally myelofibrosis, polycythemia vera (PV) or essential thrombrocythemia (ET).

In some embodiments, the condition is a neurodegenerative disorder, optionally chronic traumatic encephalopathy, Alzheimer's disease, Parkinson disease, ALS, mild or severe traumatic brain injury, blast brain injury, and the like.

In some embodiments, the condition is an inflammatory condition, optionally a cardiovascular disease such as atherosclerosis, or an autoimmune disease.

In some embodiments, the method further comprises administering a secondary therapeutic agent to the subject.

In some embodiments, the capsules (or capsular formulations) or tablets or orally administered formulations such as solutions or suspensions are administered daily, every 2 days, every 3 days, every 4 days, every 5 days, every 6 days, every week, every 2 weeks, every 3 weeks, every 4 weeks, every month, every 2 months, every 3 months, every 4 months, every 6 months, or every year. In some embodiments, the capsules (or capsular formulations) or tablets or orally administered formulations such as solutions or suspensions are administered once a day, twice a day, or thrice a day. In some embodiments, the capsules (or capsular formulations) or tablets or orally administered formulations such as solutions or suspensions are administered every 3 hours, every 4 hours, every 6 hours, every 12 hours, or every 24 hours.

Provided herein in one aspect is a method for treating a subject having a condition characterized by abnormal Hsp90 activity, presence of mis-folded proteins, or responsiveness to Hsp90 inhibition, comprising administering one or more capsules (or capsular formulations) or tablets or orally administered formulations such as solutions or suspensions comprising one or more Hsp90 inhibitors of any one of Formula I-XIV and one or more secondary therapeutic agents in a therapeutically effective amount. In some embodiments, the one or more Hsp90 inhibitors are administered or co-administered with the one or more secondary therapeutic agents.

Other advantages and novel features of the present invention will become apparent from the following detailed description of various non-limiting embodiments of the invention when considered in conjunction with the accompanying Figures. In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

### BRIEF DESCRIPTION OF DRAWINGS

Non-limiting embodiments of the present invention will be described by way of example with reference to the accompanying Figures, which are schematic and are not intended to be drawn to scale.

It is also to be understood that various Figures and exemplifications of this disclosure refer to Compound 1 as the active agent (also referred to herein as the active pharmaceutical ingredient or API). However, the disclosure intends this for illustrative purposes only and it is to be in no way limiting. Any of the Hsp90 inhibitors provided herein, such as but not limited to Compound 2, can be formulated as provided herein.

FIG. 1 is a schematic overview of the manufacturing process for Compound 1 delayed release (DR) capsules comprising minitablets.

FIG. 2 is a schematic overview of the manufacturing process for the Compound 1 dry blend capsule (non-minitablet).

FIG. 3 is a schematic overview of the manufacturing process for the Compound 1 delayed release/extended release (DR/ER) capsules comprising DR/ER minitablets.

FIG. 4 is a schematic of a delayed release/extended release (DR/ER) minitablet construct.

FIG. 5 is a schematic overview of the manufacturing process for micronization of Compound 1 to be used, for example, in hot melt granulation (HMG) capsule.

FIG. 6 is a schematic overview of the manufacturing process for hot melt high shear granulation, milling, and blending of micronized Compound 1 to be used in HMG capsules.

FIG. 7 is a schematic overview of the manufacturing process for milled granulation in-process sampling.

FIG. 8 is a schematic overview of the manufacturing process for capsule filling, dedusting, and 100% weight sorting of HMG capsules.

FIG. 9 is a flowchart of the manufacturing process for Compound 1 spray dry dispersion (SDD) tablets. The left panel illustrates the preparation of the SDD solution. The right panel illustrates the spray drying, oven drying, and in-process testing.

FIGS. 10A and 10B show schematic overviews of the manufacturing process for Compound 1 blend and encapsulation. FIG. 10A illustrates blending and in-process uniformity testing. FIG. 10B illustrates capsule filling, weight checks, dedusting, packaging and labelling of Compound 1 capsules.

FIGS. 11A and 11B show schematic overviews of the manufacturing process for Compound 1 blend and tableting. FIG. 11A (top panel) illustrates the weighing of SDI and excipients, blending/milling/blending, and in-process testing. FIG. 11A (bottom panel) illustrates roller compaction/milling, blending/milling of extra-granular excipients, extra-granular blending, blending with lubricant, and in-process testing. FIG. 11B (top panel) illustrates tablet compression, dedusting, metal detection, and weight sorting, which may be performed in parallel. FIG. 11B (bottom panel) illustrates coating, packaging and labelling.

FIG. 12 shows a schematic overview of the manufacturing process for immediate release (IR) common blend tablets of varying dosage strengths. The top panel illustrates wet granulation, wet milling and drying. The middle panel illustrates dry milling, weighing, extragranular blending, and in-process blend uniformity testing, and the bottom panel illustrates lubricant addition, final blending, milling of the specified amount of API, and allocation of formulation.

FIG. 13 shows a schematic overview of tablet compression and coating for immediate release (IR) tablets. The left panel illustrates tableting, dedusting/metal detection, weight inspection and coating. The right panel illustrates packaging.

FIG. 14 shows a schematic overview of tablet coating for delayed release (DR) tablets.

FIG. 15 shows a schematic overview of the preparation of initial granula in the wet granulation procedure.

FIG. 16 shows a schematic overview of capsule filling.

FIG. 17 shows a schematic illustrating the method of manufacture for 10 mg Compound 1 oral disintegrating tablets (ODT).

FIG. 18 shows a second schematic illustrating the method of manufacture for Compound 1 oral disintegrating tablets (ODT).

FIG. 19 shows the effect of treatment with an Hsp90 inhibitor, administered orally or intraperitoneally, on tumor volume.

FIG. 20 shows the effect of treatment with an Hsp90 inhibitor, administered orally or intraperitoneally, on body weight.

FIG. 21 shows the effect of treatment with an Hsp90 inhibitor, administered orally or intraperitoneally, on tumor volume over 36 days of treatment.

FIG. 22 shows the effect of treatment with an Hsp90 inhibitor, administered orally or intraperitoneally, on body weight over 36 days of treatment.

FIG. 23 shows the effect of treatment with an Hsp90 inhibitor, administered orally or intraperitoneally, on tumor volume over 89 days of treatment.

FIG. 24 shows the effect of treatment with an Hsp90 inhibitor, administered orally or intraperitoneally, on tumor volume during treatment and after treatment has been stopped.

FIG. 25 shows the effect of treatment with an Hsp90 inhibitor, administered orally or intraperitoneally, on body weight during treatment and after treatment has been stopped.

FIG. 26 shows the effect of three jet mill passes (P1, P2 and P3) with 51 mm collection loop on particle size distribution of Compound 2 2HCl.

FIG. 27 shows the effect of one scale up jet mill pass (P1) on particle size distribution of Compound 2 2HCl with 146 mm collection loop.

### DETAILED DESCRIPTION

This disclosure provides oral formulations for Hsp90 inhibitors. Such oral formulations will increase convenience and thus improve patient compliance during a treatment cycle, while having therapeutic efficacy at least on par with parenteral (e.g., intravenous) formulations of Hsp90 inhibitors. In addition, these oral formulations can result in improved absorption and thus bioavailability of Hsp90 inhibitors

### Oral Formulations

Oral formulations of the Hsp90 inhibitors, referred to herein as the active compounds, active ingredients, active pharmaceutical ingredients, APIs, etc., may be solid formulations or liquid formulations. Liquid formulations include but are not limited to solutions, suspensions, and emulsions, and may comprise syrups, elixirs, and the like.

Solid formulations include but are not limited to minitablets, tablets, capsules (or capsular formulations), sublingual tablets, effervescent tablets, chewable tablets, lozenges, chewing gums, wafers, and the like. A variety of manufacturing methods and thus capsule (or capsular formulation) and tablet and other oral forms are contemplated by this disclosure including but not limited to
(1) powder-filled capsules (or capsular formulations) which include
   (a) dry blend capsules,
   (b) hot melt extrusion capsules,
   (c) hot melt granulation capsules, and
   (d) spray dry dispersion (SDD) capsules, and
(2) altered release capsules (or capsular formulations) and tablets which include but are not limited to
   (a) delayed release (DR) capsules optionally comprising minitablets,
   (b) extended release (ER) capsules optionally comprising minitablets,
   (c) controlled release capsules,
   (d) sustained release capsules,
   (e) delayed release (DR) tablets,
   (f) extended release (ER) tablets, and
   (g) controlled release tablets, and
   (h) sustained release capsules,
(3) tablets which include
   (a) dry blend tablets
   (b) hot melt extrusion tablets,
   (c) hot melt granulation tablets,
   (d) spray dry dispersion (SDD) tablets,
   (e) wet granulation—dry blend tablets
   (f) oral disintegrating tablets (ODT), and
   (g) uncoated or coated tablets, including enterically coated tablets.

As used herein, a capsular formulation is a formulation that comprises a capsule. The capsule may or may not comprise minitablets.

The oral formulations provided herein comprise a therapeutically effective amount of one or more active compounds disclosed herein. The term “therapeutically effective amount” refers to an amount of an active compound or a combination of two or more compounds that inhibits, totally or partially, the progression of the condition being treated or alleviates, at least partially, one or more symptoms of the condition. For example, the compounds may be an Hsp90 inhibitor and a second therapeutic agent, and in some embodiments the therapeutically effective amount is the amount of these two classes of agents when used together (including for example the amount of each class of agent). A therapeutically effective amount can also be an amount which is prophylactically effective when given, for example, to a subject at risk of developing the condition or a subject who has been successfully treated but may be at risk of a recurrence. The amount which is therapeutically effective depends on the patient's gender and size, the condition to be treated, the condition's severity, and the result sought. For a given patient, a therapeutically effective amount can be determined by methods known to those in the art.

Dosage strength, as used herein, refers to the amount of active compound in a single dose oral formulation (e.g., a single capsule, or a single tablet, etc.). Dosages may range from about 0.001 to about 1000 mg, including about 0.01 mg to about 1000 mg, including 0.01 mg to about 1000 mg, including about 1 mg to about 1000 mg of Hsp90 inhibitor. Exemplary dosage strengths include at least 0.001, at least 0.005, at least 0.01, at least 0.05, at least 0.1, at least 0.5, at least 1 mg, at least 2 mg, at least 3 mg, at least 4 mg, at least 5 mg, at least 10 mg, at least 15 mg, at least 20 mg, at least 25 mg, at least 30 mg, at least 35 mg, at least 40 mg, at least 45 mg, at least 50 mg, at least 55 mg, at least 60 mg, at least 65 mg, at least 70 mg, at least 75 mg, at least 80 mg, at least 85 mg, at least 90 mg, at least 95 mg, at least 100 mg, at least 125 mg, at least 150 mg, at least 175 mg, at least 200 mg, at least 300 mg, at least 400 mg, at least 500 mg or more of Hsp90 inhibitor. Exemplary dosage strengths include 0.001, 0.005, 0.01, 0.05, 0.1, 0.5, 1 mg, 2 mg, 3 mg, 4 mg, 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 125 mg, 150 mg, 175 mg, 200 mg, 300 mg, 400 mg, 500 mg, or more, of Hsp90 inhibitor, including all doses therebetween as is explicitly recited herein. In some instances, when a large dose is required, several of a smaller dosage form may be administered or a single larger dosage form may be administered.

The oral formulations provided herein (e.g., minitablets, capsules (or capsular formulations) and tablets and orally administered formulations such as solutions or suspensions) may be administered daily, every 2 days, every 3 days, every 4 days, every 5 days, every 6 days, every week, every 2 weeks, every 3 weeks, every 4 weeks, every month, every 2 months, every 3 months, every 4 months, every 6 months, or every year.

The oral formulations provided herein may be administered for a period of time (referred to as a treatment period) followed by a period of time in which the oral formulations are not administered to the subjects (referred to herein as a non-treatment period). The treatment period may be 1, 2, 3, 4, 5, 6 or 7 days and the non-treatment period may be 1, 2, 3, 4, 5, 6, or 7 or more days. Alternatively, the treatment period may be 1, 2, 3 or 4 weeks and the non-treatment period may be 1, 2, 3, 4 or more weeks. The non-treatment period may be as long as or 2, 3, 4, 5, 6, 7, 8, 9 or 10 times as long as the treatment period. The treatment and non-treatment periods may be repeated 1, 2, 3, 4, 5, 6, 7, 8, 9 or 10 or more times. In some embodiments, the treatment period is 1 week and the non-treatment period is 3 weeks, and these are repeated 1, 2, 3, 4, 5, 6, 7, 8, 9 or 10 or more times.

The oral formulations provided herein may be administered once a day, twice a day, or thrice a day. The oral formulations provided herein may be administered every 3 hours, every 4 hours, every 6 hours, every 12 hours, or every 24 hours.

### Hsp90 Inhibitors

For the sake of brevity, the term Hsp90 will be used herein to collectively refer to Hsp90, its isoforms and its homologs such as but not limited to GRP94 and TRAP1. Thus, the Hsp90 inhibitors of this disclosure inhibit Hsp90 and/or Hsp90 isoforms and/or Hsp90 homologs including but not limited to GRP94 and TRAP1. Again for the sake of brevity, inhibitors of Hsp90 (Hsp90-alpa and Hsp90-beta in the cytoplasm), Hsp90 isoforms and Hsp90 homologs, such as but not limited to GRP94 (a form of Hsp90 found in the endoplasmic reticulum) and TRAP1 (a form of Hsp90 found in the mitochondria), are referred to herein collectively as Hsp90 inhibitors.

The disclosure also provides Hsp90 inhibitors that interfere with the formation or stability of the epichaperome, thereby rendering target cells (such as cancer cells) more susceptible to cell death. The ability to target the epichaperome can also result in reduced general toxicity in subjects being treated. Accordingly, the inhibitors of this disclosure may also be referred to as epichaperome inhibitors.

One class of Hsp90 inhibitors of this disclosure are purine-scaffold compound having the general structure of Formula I:

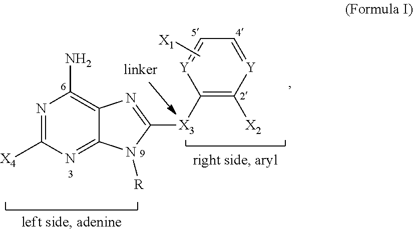

wherein each Y is independently chosen as C, N or O, with the proviso that when Y is O the double bonds are missing or rearranged to retain the aryl nature of the ring, optionally wherein both Y are C or N or O in some instances,

R is hydrogen, a C1 to C10 alkyl, alkenyl, alkynyl, or an alkoxyalkyl group, optionally including heteroatoms such as N or O, or a targeting moiety connected to N9 via a linker,

X4 is hydrogen or halogen, for example F or Cl, or Br;

X3 is CH2, CF2 S, SO, SO2, O, NH, or NR2, wherein R2 is alkyl; and

X2 is halogen, alkyl, alkoxy, halogenated alkoxy, hydroxyalkyl, pyrollyl, optionally substituted aryloxy, alkylamino, dialkylamino, carbamyl, amido, alkylamido dialkylamido, acylamino, alkylsulfonylamido, trihalomethoxy, trihalocarbon, thioalkyl, SO2.alkyl, COO-alkyl, NH2, OH, CN, SO2X5, NO2, NO, C═S R2, NSO2X5, C═OR2, where X5 is F, NH2, alkyl or H, and R2 is alkyl, NH2, NH-alkyl or O-alkyl; and

X1 represents two substituents, which may be the same or different, disposed in the 4′ and 5′ positions on the aryl group, wherein X1 is selected from halogen, alkyl, alkoxy, halogenated alkoxy, hydroxyalkyl, pyrollyl, optionally substituted aryloxy, alkylamino, dialkylamino, carbamyl, amido, alkylamido dialkylamido, acylamino, alkylsulfonylamido, trihalomethoxy, trihalocarbon, thioalkyl, SO2.alkyl, COO-alkyl, NH2, OH, CN, SO2X5, NO2, NO, C═SR2 NSO2X5, C═OR2, where X5 is F, NH2, alkyl or H, and R2 is alkyl, NH2, NH-alkyl or O-alkyl, C1 to C6 alkyl or alkoxy; or wherein X1 has the formula -0-(CH2)n-0-, wherein n is an integer from O to 2, and one of the oxygens is bonded at the 5′-position and the other at the 4′-position of the aryl ring.

The right-side aryl group may be phenyl as shown, or may include one or more heteroatoms. For example, the right-side aryl group may be a nitrogen-containing aromatic heterocycle such as pyrimidine.

In specific preferred embodiments of the composition of the invention, the right side aryl group X1 has the formula -0-(CH2)n-0-, wherein n is an integer from 10 to 2, preferably 1 or 2, and one of the oxygens is bonded at the 5′-position of the aryl ring and the other at the 4′ position. In other specific embodiments of the invention, the substituents X1 comprise alkoxy substituents, for example methoxy or ethoxy, at the 4′ and 5′-positions of the aryl ring.

In specific embodiments of the invention, the substituent X2 is a halogen.

In specific embodiments of the invention, the linker X3 is S. In other specific embodiments of the invention, the linker X3 is CH2.

In specific embodiments of the invention, R is a pent-4-ynyl substituent. In other specific embodiments of the invention, R contains a heteroatom, for example nitrogen. A preferred R group that increases the solubility of the compound relative to an otherwise identical compound in which R is H or pent-4-ynyl is —(CH2Xn-N—R10R11R12, where m is 2 or 3 and where R10.12 are independently selected from hydrogen, methyl, ethyl, ethene, ethyne, propyl, isopropyl, isobutyl, ethoxy, cyclopentyl, an alkyl group forming a 3 or 6-membered ring including the N, or a secondary or tertiary amine forming a 6-membered ring with the nitrogen. In specific examples, R10 and R11 are both methyl, or one of R10 and Rn is methyl and the other is ethyne.

Another class of Hsp90 inhibitors of this disclosure are purine scaffold compounds having the general structure of Formula II:

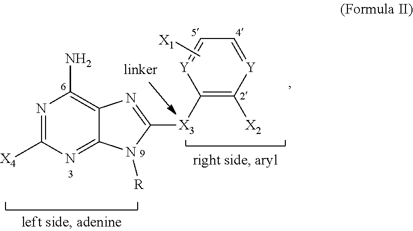

wherein R is hydrogen, a C1 to C10 alkyl, alkenyl, alkynyl, or an alkoxyalkyl group, optionally including heteroatoms such as N or O, optionally connected to the 2′-position to form an 8 to 10 member ring:

wherein the Ys are regarded as Y1 and Y2 that are independently selected as C, N, S or O, with the proviso that when Y1 and/or Y2 is O the double bonds are missing or rearranged to retain the aryl nature of the ring,

X4 is hydrogen, halogen, for example F or Cl, or Br;

X3 is CH2, CF2 S, SO, SO2, O, NH, or NR2, wherein R2 is alkyl; and

X2 is halogen, alkyl, halogenated alkyl, alkoxy, halogenated alkoxy, hydroxyalkyl, pyrollyl, optionally substituted aryloxy, alkylamino, dialkylamino, carbamyl, amido, alkylamido dialkylamido, acylamino, alkylsulfonylamido, trihalomethoxy, trihalocarbon, thioalkyl, SO2 alkyl, COO-alkyl, NH2 OH, or CN or part of a ring formed by R; and

X1 represents one more substituents on the aryl group, with the proviso that X1 represents at least one substituent in the 5′-position said substituent in the 5′-position being selected from the same choices as X2 C1 to C6alkyl or alkoxy; or wherein X1 has the formula —O—(CH2)-O—, wherein n is 1 or 2, and one of the oxygens is bonded at the 5′-position of the aryl ring and the other is bonded to the 4′ position.

The ride-side aryl group may be phenyl, or may include one or more heteroatoms. For example, the right-side aryl group may be a nitrogen-containing aromatic heterocycle such as pyrimidine.

In specific embodiments of the composition of the invention, the right-side aryl group is substituted at the 2′ and 5′ position only. In other embodiment, the right side aryl group is substituted at the 2′, 4′, and 5′ positions. In yet other embodiments, the right side aryl group is substituted at the 4′ and 5′ positions only. As will be appreciated by persons skilled in the art, the numbering is based on the structure as drawn, and variations in the structure such as the insertion of a heteroatom may alter the numbering for purposes of formal nomenclature.

In other specific embodiments of the composition of the invention, the right side aryl group has a substituent at the 2′-position and X1 has the formula -X-Y-Z- with X and Z connected at the 4′ and 5′ positions to the right side aryl, wherein X, Y and Z are independently C, N, S or O, connected by single or double bonds and with appropriate hydrogen, alkyl or other substitution to satisfy valence. In some embodiments, at least one of X, Y and Z is a carbon atom. In one specific embodiment, X1 is -0-(CH2)n-0-, wherein n is 1 or 2, and one of the oxygen atoms is bonded at the 5′-position of the aryl ring and the other at the 4′ position.

In some embodiments, the compound had the structure of Formula III:

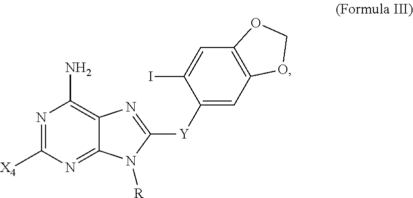

wherein:

Y is —CH2- or S,

X₄ is hydrogen or halogen and

R is an amino alkyl moiety, optionally substituted on the amino nitrogen with one or two carbon-containing substituents selected independently from the group consisting of alkyl, alkenyl and alkynyl substituents, wherein the total number of carbons in the amino alkyl moiety is from 1 to 9, and wherein the compound is optionally in the form of an acid addition salt.

In some embodiments, R is —(CH₂)m-N—R₁₀R₁₁m, where m is 2 or 3, and R₁₀ and R₁₁ are independently selected from hydrogen, methyl, ethyl, ethenyl, ethynyl, propyl, isopropyl, t-butyl and isobutyl. In some embodiments, Y is S.

In some embodiments, R is selected from the group consisting of 2-(methyl, t-butyl amino)ethyl, 2-(methyl, isopropyl amino)ethyl, 2-(ethyl, isopropyl amino)ethyl, 3-(isopropyl amino) propyl, 3-(t-butyl amino) propyl, 2-(isopropyl amino)ethyl, 3-(ethylamino) propyl, and 3-(ethyl, methyl amino) propyl.

In some embodiments, I in the compound is ¹²⁴I, ¹³¹I, or ¹²³I.

In some embodiments, I in the compound is ¹²⁷I (i.e., non-radioactive iodine).

In some embodiments, the compound has the structure:

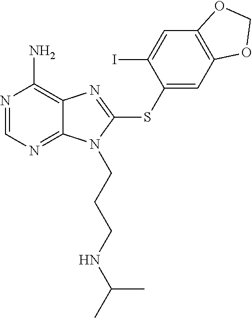

wherein I is ¹²⁷I (referred to herein as Compound 1).

In some embodiments, the compound has the structure:

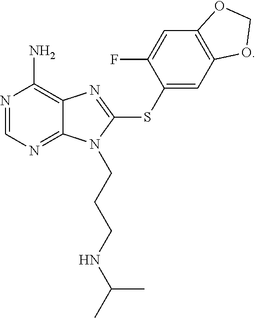 [SMILES: CC(C)NCCCn1c(Sc2cc3c(cc2F)OCO3)nc2c(N)ncnc21]

In some embodiments, the F in the foregoing compound is ¹⁸F, and such compound is referred to herein as Compound 1a.

Another class of Hsp90 inhibitors of this disclosure have the general structure of Formula IV:

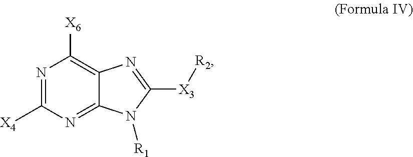

or an acid addition salt thereof,
wherein X₄ is hydrogen or halogen;

X₆ is amino;

X₃ is C, O, N, or S with hydrogens as necessary to satisfy valence, or CF₂, SO, SO₂ or NR₃ where R₃ is alkyl;

R₁ is selected from the group consisting of 3-((2-hydroxyethyl)(isopropyl)amino)propyl, 3-(methyl(prop-2-ynyl)amino)propyl, 3-(allyl(methyl)amino)propyl, 3-(cyclohexyl(2-hydroxyethylamino)propyl, 3-(4-(2-hydroxyethyl)piperazin-1-yl)propyl, 2-(isopropylamino)ethyl, 2-(isobutylamino)ethyl, or 2-(neopentylamino)ethyl, 2-(cyclopropylmethylamino)ethyl, 2-(ethyl(methyl)amino)ethyl, 2-(isobutyl(methyl)amino)ethyl, and 2-(methyl(prop-2-ynyl)amino)ethyl, or an acid addition salt thereof; and

### R₂ is

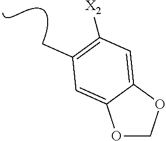

wherein X₂ is halogen.

Another class of Hsp90 inhibitors of this disclosure have the general structure of Formula V:

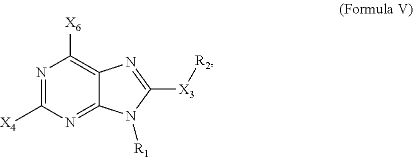

or an acid addition salt thereof,
wherein X₄ is hydrogen or halogen;

X₆ is amino;

X₃ is C, O, N, or S with hydrogens as necessary to satisfy valence, or CF₂, SO, SO₂ or NR₃ where R₃ is alkyl;

R₁ is 2-(isobutylamino)ethyl or 2-(neopentylamino)ethyl, or an acid addition salt thereof; and

### R₂ is

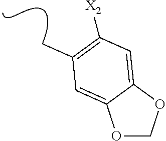

wherein X₂ is halogen.

In some embodiments, R1 is 2-(neopentylamino)ethyl.

In some embodiments, R1 is 2-(isobutylamino)ethyl.

In some embodiments, the compound has the structure:

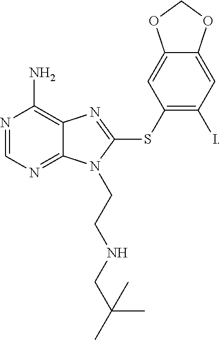 [SMILES: CC(C)(C)CNCCn1c(Sc2cc3c(cc2I)OCO3)nc2c(N)ncnc21]

In some embodiments, I in the foregoing compound is ¹²⁴I, ¹³¹I, or ¹²³I.

In some embodiments, I in the foregoing compound is ¹²⁷I (i.e., non-radioactive iodine), and the compound is referred to as Compound 2.

In some embodiments, the compound has the structure:

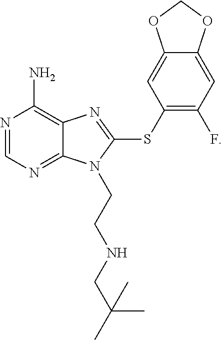 [SMILES: CC(C)(C)CNCCn1c(Sc2cc3c(cc2F)OCO3)nc2c(N)ncnc21]

In some embodiments, F in the foregoing compound is ¹⁸F, and the compound is referred to as Compound 2a.

Another class of Hsp90 inhibitors of this disclosure have the general structure of Formula VI:

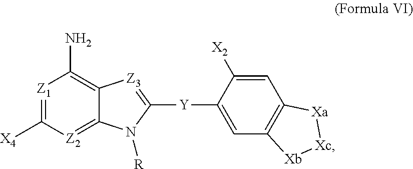

wherein
(a) each of Z1, Z2 and Z3 is independently C or N, with H substituents as needed to satisfy valence;
(b) Xa, Xb and Xc are all carbon (C), connected by two single or one single bond and one double bond,

### (c) Y is —CH2- or —S—;

(d) X4 is hydrogen or halogen; and
(e) X2 and R in combination are selected from the group consisting of:
(i) X2 is halogen and R is primary amino-alkyl, a secondary or tertiary alkyl-amino-alkyl, aryl-alkyl, or a nonaromatic heterocycle-alkyl, wherein the amine's nitrogen and the heterocycle's heteroatom are substituted to satisfy valence, with the proviso that R is not a piperidine moiety; and
(ii) X2 is selected from the group consisting of alkyl, alkenyl, alkynyl, aryl, cycloalkyl, cycloalkenyl, saturated or unsaturated heterocycle, aryl, aryloxy, alkoxy, halogenated alkoxy, alkenyloxy, hydroxyalkyl, amino, alkylamine, dialkylamino, acylarino, carbamyl, amido, dialkylamido, alkylamido, alkylsulfonamido, sulfonarnido, trihalocarbon, -thioalkyl, SO2-alkyl, —COO-alkyl, OH or alkyl-CN, or part of a ring formed by R, and R is a group as listed below in Table A.

Another class of Hsp90 inhibitors of this disclosure have the general structure of Formula Via:

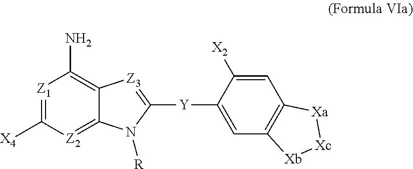

wherein

(a) each of Z1, Z2 and Z3 is independently C or N, with H substituents as needed to satisfy valence;

(b) Xa, Xb and Xc are all carbon, connected by two single or one single bond and one double bond, and wherein

(c) Y is —CH₂— or —S—;

(d) X4 is hydrogen or halogen; and

(e) X₂ and R in combination are selected from the group consisting of:
(i) X₂ is halogen and R is primary amino-alkyl, a secondary or tertiary alkyl-amino-alkyl, aryl-alkyl, or a nonaromatic heterocycle-alkyl, wherein the amine's nitrogen and the heterocycle's heteroatom are substituted to satisfy valence, with the proviso that R is not a piperidino moiety; and
(ii) X₂ is selected from the group consisting of alkyl, alkenyl, alkynyl, aryl, cycloalkyl, cycloalkenyl, saturated or unsaturated heterocycle, aryl, aryloxy, alkoxy, halogenated alkoxy, alkenyloxy, hydroxyalkyl, amino, alkylamino, dialkylamino, acylamino, carbamyl, amido, dialkylamido, alkylamido, alkylsulfonamido, sulfonamido, trihalocarbon, -thioalkyl, S0₂-alkyl, —COO-alkyl, OH or alkyl-CN, or part of a ring formed by R, and

R is a group listed in Table A.

In some embodiments of Formula VIa, X₂ is not halogen.

In some embodiments of Formula VIa, X₂ is alkynyl.

In some embodiments of Formula VIa, the compound is selected from the group consisting of: 8-((6-ethynyl-2,3-dihydro-1H-inden-5-yl)thio)-9-(3-(isopropylamino)propyl)-9H-purin-6-amine; 1-(3-(2-(6-amino-8-(6-ethynyl-2,3-dihydro-1H-inden-5-ylthio)-9H-purin-9-yl)ethyl)piperidin-1-yl)ethanone; 1-(3-(3-(6-amino-8-(6-ethynyl-2,3-dihydro-1H-inden-5-ylthio)-9H-purin-9-yl)propyl)pyrolidin-1-yl)ethanone; 8-((6-ethynyl-2,3-dihydro-1H-inden-5-yl)thio)-9-(2-(neopentylamino)ethyl)-9H-purin-6-amine; 5-(6-amino-8-(6-ethynyl-2,3-dihydro-1H-inden-5-ylthio)-9H-purin-9-yl)pentane-1-sulfonamide; 1-(4-(3-(6-amino-8-(6-ethynyl-2,3-dihydro-1H-inden-5-ylthio)-9H-purin-9-yl)propyl)piperidin-1-yl)ethanone; 9-(3-(tert-butylamino)propyl)-8-(6-ethynyl-2,3-dihydro-1H-inden-5-ylthio)-9H-purin-6-amine; 1-acetyl-3-(3-(6-amino-8-(6-ethynyl-2,3-dihydro-1H-inden-5-ylthib)-9H-purin-9-yl)propyl)imidazolidin-2-one; 8-((6-ethynyl-2,3-dihydro-1H-inden-5-yl)thio)-9-(2-(1-methylpiperidin-2-yl)ethyl)-9H-purin-6-amine; 8-((6-ethynyl-2,3-dihydro-1H-inden-5-yl)thio)-9-(2-(1-methylpiperidin-3-yl)ethyl)-9H-purin-6-amine; 8-((6-ethynyl-2,3-dihydro-1H-inden-5-yl)thio)-9-(2-(1-(methylsulfonyl)piperidin-3-yl)ethyl)-9H-purin-6-amine; 1-(3-(2 6-amino-8-((6-ethynyl-2,3-dihyo\hat{ }o H-inden-5-yl)methyl)-2-fluoro-9H-purin-9-yl)ethyl)piperidin-1-yl)ethanone; 9-(3-(tert-butylamino)propyl)-8-((6-ethynyl-2,3-dihydro-1H-inden-5-yl)methyl)-2-fluoro-9H-purin-6-amine; 6-(6-amino-8-((6-ethynyl-2,3-dihydro-1H-inden-5-yl)methyl)-2-fluoro-9H-purin-9-yl)hexanamide; 1-(3-(6-amino-8-((6-ethynyl-2,3-dihydro-1H-inden-5-yl)methyl)-2-fluoro-9H-purin-9-yl)propyl)pyrrolidin-3-one; 4-(6-amino-8-((6-ethynyl-2)3-dihydro-1H-inden-5-yl)methyl)-2-fluoro-9H-purin-9-yl)butane-1-sulfonamide; 8-((6-ethynyl-2,3-dihydro-1H-inden-5-yl)methyl)-2-fiuoro-9-(3-(isopropylamino)propyl)-9H-purin-6-amine; 8-((6-ethynyl-2,3-dihydro-1H-inden-5-yl)methyl)-2-fluoro-9-(2-(neopentylamino)ethyl)-9H-purin-6-amine; 3-(2-(6-amino-8-((6-ethynyl-2,3-dihydro-1H-inden-5-yl)methyl)-2-fluoro-9H-purin-9-yl)ethyl)piperidine-1-sulfonamide; 8-((6-ethynyl-2,3-dihydro-1H-inden-5-yl)methyl)-2-fluoro-9-(2-(1-methylpiperidin-2-yl)ethyl)-9H-purin-6-amine; and 8-((6-ethynyl-2,3-dihydro-1H-inden-5-yl)methyl)-2-fluoro-9-(2-(1-methylpiperidin-3-yl)ethyl)-9H-purin-6-amine

In some embodiments of Formula VIa, X2 is heteroaryl.

In some embodiments of Formula VIa, the compound is selected from the group consisting of: 8-((6-(furan-2-yl)-2,3-dihydro-1H-inden-5-yl)thio)-9-(3-(isopropylamino)propyl)-9H-purin-6-amine; 9-(3-(isopropylamino)propyl)-8-((6-(oxazol-2-yl)-2,3-dihydro-1H-inden-5-yl)thio)-9H-purin-6-amine; 1-(3-(2-(6-amino-8-(6-(oxazol-2-yl)-2,3-dihydro-1H-inden-5-ylthio)-9H-purin-9-yl)ethyl)piperidin-1-yl)ethanone; 3-(2-(8-(6-(1H-pyrazol-3-yl)-2,3-dihydro-1H-inden-5-ylthio)-6-arrimo-9H-purin-9-yl)ethyl)pipericarbaldehyde; N-(2-((2-(6-amino-8-((6-(oxazol-2-yl)-2,3-dihydro-1H-inden-5-yl)thio)-9H-purin-9-yl)ethyl)amino)ethyl)sulfamide; 3-(2-(6-amino-8-(6-(oxazol-2-yl)-2,3-dihydro-1H-inden-5-ylthio)-9H-purin-9-yl)ethylamino)-N-hydroxypropanamide; 9-(3-(isopropylamino)propyl)-8-((6-(5-methyloxazol-2-yl)-2,3-dihydro-1H-inden-5-yl)thio)-9H-purin-6-amine; 8-((6-(5-methyloxazol-2-yl)-2,3-dihydro-1H-inden-5-yl)thio)-9-(2-(1-(methylsulfonyl)piperidin-3-yl)ethyl)-9H-purin-6-amine; 9-(3-aminopropyl)-8-((6-(5-methyloxazol-2-yl)-2,3-dihydro-1H-inden-5-yl)thio)-9H-purin-6-amine; 9-(3-(tert-bulylamino)propyl)-8-(6-(4-memyltm\hat{ }ol-2-yl)-2,3-dihydro-1H-inden-5-ylthio)-9H-purin-6-amine; 8-((6-(5-methyloxazol-2-yl)-2,3-dihydro-1H-inden-5-yl)thio)-9-(2-(neopentylaniino)ethyl)-9H-purin-6-amine; 1-(6-amino-8-((6-(5-methyloxazol-2-yl)-2,3-dihydro-1H-inden-5-yl)thio)-9H-purin-9-yl)-3-(isopropylamino)propan-2-ol; 1-(2-(4-(6-amino-8-(6-(5-methylfuran-2-yl)-2,3-dihydro-1H-inden-5-ylthio)-9H-purin-9-yl)butyl)pyrrolidin-1-yl)ethanone; 1-(3-(2-(6-amino-8-(6-(5-methyloxazol-2-yl)-2,3-dihydro-1H-inden-5-ylthio)-9H-purin-9-yl)ethyl)piperidin-1-yl)ethanone; 6-(6-amino-8-(6-(oxazol-2-yl)-2,3-dihydro-1H-inden-5-ylthio)-9H-purin-9-yl)hexanamide; 1-(3-(6-amino-8-(6-(4-methyloxa2ol-2-yl)-2,3-dihydro-1H-inden-5-ylthio)-9H-purin-9-yl)propyl)pyrrolidin-1-3-one; 2-fiuoro-9-(3-(1-(methylsulfonyl)pyrrolidin-3-yl)propyl)-8-((6-(oxazol-2-yl)-2,3-dihydro-1H-inden-5-yl)methyl)-9H-purin-6-amine; 1-(3-(2-(6-amino-2-fluoro-8-((6-(4-methylthiazol-2-yl)-2,3-dihydro-1H-inden-5-yl)methyl)-9H-purin-9-yl)ethyl)piperidin-1-yl)ethanone; 9-(3-(tert-butylamino)propyl)-2-fluoro-8-((6-(4-memylthiazol-2-yl)-2,3-dihydro-1H-inden-5-yl)methyl)-9H-purin-6-amine; 8-((6-(1H-pyrazol-3-yl)-2,3-dihydro-1H-inden-5-yl)methyl)-9-(3-(tert-butylarmno)propyl)-2-fluoro-9H-purin-6-arnine; 6-(6-amino-2-fluoro-8-((6-(oxazol-2-yl)-2,3-dihydro-1H-inden-5-yl)methyl)-9H-purin-9-yl)hexanamide; 1-(3-(6-amino-2-fluoro-8-((6-(oxazol-2-yl)-2,3-dihydro-1H-inden-5-yl)methyl)-9H-purin-9-yl)propyl)pyrrolidin-3-one; 5-(6-amino-2-fluoro-8-((6-(oxazol-2-yl)-2,3-dihydro-1H-inden-5-yl)methyl)-9H-purin-9-yl)pentane-1-sulfonamide; 2-fluoro-9-(2-(1-methylpiperidin-2-yl)ethyl)-8-((6-(oxazol-2-yl)-2,3-dihydro-1H-inden-5-yl)methyl)-9H-purin-6-amine; and 2-fiuoro-9-(2-(1-methylpiperidin-3-yl)ethyl)-8-((6-(oxazol-2-yl)-2,3-dihydro-1H-inden-5-yl)methyl)-9H-purin-6-amine.

In some embodiments of Formula VIa, X₂ is iodine.

In some embodiments, the Hsp90 inhibitor is selected from the group consisting of: 1-(6-amino-8-(6-iodo-2,3-dihydro-1H-inden-5-ylthio)-9H-purin-9-yl)-3-(tert-butylamino)propan-2-ol; 8-((6-iodo-2,3-dihydro-1H-inden-5-yl)thio)-9-(2-(isobutylamino)ethyl)-9H-purin-6-amine; 1-(3-(6-amino-8-(6-iodo-2,3-dihydro-1H-inden-5-ylthio)-9H-purm-9-yl)propyl)pyrrolidin-3-one; 1-(3-(3-(6-amino-8-(6-iodo-2,3-dihydro-1H-inden-5-ylthio)-9H-purin-9-yl)propyl)pyrrolidin-1-yl)ethanone; 8-((6-iodo-2,3-dihydro-1H-inden-5-yl)thio)-9-(2-(neopentylamino)ethyl)-9H-purin-6-amine; 8-((6-iodo-2,3-dihydro-1H-inden-5-yl)thio)-9-(3-(isopropylamino)propyl)-9H-purin-6-amine; 9-(3-aminopropyl)-8-((6-iodo-2,3-dihydro-1H-inden-5-yl)thio)-9H-purin-6-amine; 9-(2-aminoethyl)-8-((6-iodo-2,3-dihydro-1H-inden-5-yl)thio)-9H-purin-6-amine; 9-(3-(tert-butylamino)propyl)-8-((6-iodo-2,3-dihydro-1H-inden-5-yl)thio)-9H-purin-6-amine; 5-(6-amino-8-(6-iodo-2,3-dihydro-1H-inden-5-ylthio)-9H-purin-9-yl)-N-methylpentane-1-sulfonamide; 5-(6-amino-8-(6-iodo-2,3-dihydro-1H-inden-5-ylthio)-9H-purin-9-yl)pentane-1-sulfonamide; 1-(3-(6-amino-8-(6-iodo-2,3-dihydro-1H-inden-5-ylthto)-9H-purin-9-yl)propyl)pyrolidin-3-ol; 6-(6-amino-8-(6-iodo-2,3-dihydro-1H-inden-5-ylthio)-9H-purin-9-yl)hexanamide; 8-((6-iodo-2,3-dihydro-1H-inden-5-yl)thio)-9-(2-(1-methylpiperidin-2-yl)ethyl)-9H-purin-6-amine; 8-((6-iodo-2,3-dihydro-1H-inden-5-yl)thio)-9-(2-(1-methylpiperidin-3-yl)ethyl)-9H-purin-6-anine; 8-((6-iodo-2,3-dihydro-1H-inden-5-yl)thio)-9-(2-(1-(methylsulfonyl)piperidin-3-yl)ethyl)-9H-purin-6-amine; 3-(2-(6-amino-8-((6-iodo-2,3-dihydro-1H-inden-5-yl)thio)-9H-purin-9-yl)ethyl)piperidine-1-sulfonamide; 2-fiuoro-8-((6-iodo-2,3-dihydro-1H-inden-5-yl)methyl)-9-(2-(isobutylamino)ethyl)-9H-purin-6-amine; 2-fluoro-8-((6-iodo-2,3-dihydro-1H-inden-5-yl)methyl)-9-(3-(isopropylamino)propyl)-9H-purin-6-amine; 1-(3-(6-amino-2-fluoro-8-((6-iodo-2,3-dihydro-1H-inden-5-yl)methyl)-9H-purin-9-yl)pr0pyl)pyiToli 1-(3-(3-(6-amino-2-fluoro-8-((6-iodo-2,3-dihydro-1H-inden-5-yl)methyl)-9H-purin-9-yl)propyl)pyrrolidin-1-yl)ethanone; 9-(3-(ter-butylamino)propyl)-2-fluoro-8-((6-iodo-2,3-dihydro-H-inden-5-yl)methyl)-9H-purin-6-amine; 5-(6-amino-2-fiuoro-8-((6-iodo-2,3-dihydro-1H-inden-5-yl)methyl)-9H-purin-9-yl)-N-methylpentane-1-sulfonamide; 5-(6-amino-2-fluoro-8-((6-iodo-2,3-dihydro-1H-inden-5-yl)methyl)-9H-purin-9-yl)pentane-1-sulfonamide; 2-fluoro-8-((6-iodo-2,3-dihydro-1H-inden-5-yl)methyl)-9-(2-(1-methylpiperidin-2-yl)ethyl)-9H-purin-6-amine; 2-fluoro-8-((6-iodo-2,3-dihydro-1H-inden-5-yl)methyl)-9-(2-(1-methylpiperidin-3-yl)ethyl)-9H-purin-6-amine; 2-fluoro-8-((6-iodo-2,3-dihydro H-inden-5-yl)methyl)-9-(2-(1-(methylsulfonyl)piperidin-3-yl)ethyl)-9H-purin-6-amine; 3-(2-(6-amino-2-fluoro-8-((6-iodo-2,3-dihydro-1H-inden-5-yl)methyl)-9H-purin-9-yl)ethyl)piperidine-1-sulfonamide; and 9-(3-(tert-butylamino)propyl)-2-fluoro-8-((6-iodo-2,3-dihydro-1H-inden-5-yl)methyl)-9H-purin-6-amine

Another class of Hsp90 inhibitors of this disclosure have the general structure of Formula VII:

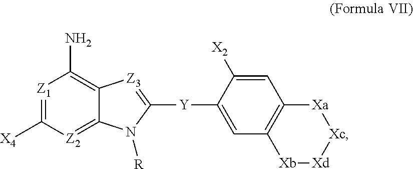

wherein

(a) each of Z1, Z2 and Z3 is independently C or N, with H substituents as needed to satisfy valence;

(b) Xa and Xb are O, and Xc and Xd are CH₂;

(c) Y is —CH₂—, —O— or —S—;

(d) X4 is hydrogen or halogen; and

(e) X₂ and R are a combination selected from:
(i) X₂ is halogen or cyano and R is suitably a primary amino alkyl, a secondary or tertiary alkyl-amino-alkyl, a trialkylammonioalkyl group, an aryl-alkyl, or a nonaromatic heterocycle-alkyl, with the proviso that R does not include a piperidino moiety; and
(ii) X₂ is selected from the group consisting of an aryl, an alkynyl, a cycloalkyl and an cycloalkenyl; and

R is a group listed in Table A.

In some embodiments of Formula VII, X₂ is halogen.

In some embodiments of Formula VII, X₂ is iodine.

In some embodiments, the Hsp90 inhibitor is selected from the group consisting of: 8-((7-iodo-2,3-dihydrobenzo[b][1,4]dioxin-6-yl)thio)-9-(3-(isopropylamino)propyl)-9H-purin-6-amine; 8-((7-iodo-2,3-dihydrobenzo[b][1,4]dioxin-6-yl)thio)-9-(2-(isobutylamino)ethyl)-9H-purin-6-amine; 8-((7-iodo-2,3-dihydrobenzo[b][1,4]dioxin-6-yl)thio)-9-(2-(neopentylann\hat{ }o)emyl)-9H-purm-6-amine; 9-(3-(1H-imidazol-1-yl)propyl)-8-((7-iodo-2,3-dihydrobenzo[b][1,4]dioxin-6-yl)thio)-9H-purin-6-amine; 9-(3-aminopropyl)-8-((7-iodo-2,3-dihydrobenzo[b][1,4]dioxin-6-yl)thio)-9H-purin-6-amine; 9-(2-aminoethyl)-8-((7-iodo-2,3-dihydrobenzo[b][1,4]dioxin-6-yl)thio)-9H-purin-6-amine; 9-(3-(tert-butylarmno)propyl)-8-((7-iodo-2,3-dihydrobenzo[b][1,4]dioxin-6-yl)thio)-9H-purin-6-amine; 1-(6-amino-8-((7-iodo-2,3-dihydrobenzo[b][1,4]dioxin-6-yl)thio)-9H-purin-9-yl)-3-(isopropylamino)propan-2-ol; 5-(6-amino-8-(7-iodo-2,3-dihydrobenzo[b][1,4]dioxin-6-ylthio)-9H-purin-9-yl)pentane-1-sulfonamide; 1-(3-(6-amino-8-(7-iodo-2,3-dihydrobenzo[b][1,4]dioxin-6-ylthio)-9H-purin-9-yl)propyl)pyiTolidin-3-one; 6-(6-amino-8-(7-iodo-2,3-dihydrobenzo[b][1,4]dioxin-6-ylthio)-9H-purin-9-yl)hexanamide; 1-(3-(4-(6-amino-8-(7-iodo-2,3-dihydrobenzo[b][1,4]dioxin-6-ylthio)-9H-purin-9-yl)butyl)pyrrolidin-1-yl)ethanone; and 8-(7-iodo-2,3-dihydrobenzo[b][1,4]dioxin-6-ylthio)-9-(3-(isobutylamino)propyl)-9H-purin-6-amine.

In some embodiments of Formula VII, X₂ is heteroaryl. In some embodiments of Formula VII, X₂ is pyrazole.

In some embodiments, the Hsp90 inhibitor is selected from the group consisting of: 8-((7-(1H-pyrazol-3-yl)-2,3-dihydrobenzo[b][1,4]dioxin-6-yl)thio)-9-(3-(isopropylamino)propyl)-9H-purin-6-amine; 8-((7-(1H-pyrazol-3-yl)-2,3-dihydrobenzo[b][1,4]dioxin-6-yl)thio)-9-(2-(neopentylamino)ethyl)-9H-purin-6-amine; 1-(4-(2-(8-((7-(1H-pyrazol-3-yl)-2,3-dihydrobenzo[b][1,4]dioxin-6-yl)thio)-6-amino-9H-purin-9-yl)ethyl)piperidin-1-yl)ethanone; 8-(7-(1H-pyrazol-3-yl)-2,3-dihydrobenzo[b][1,4]dioxin-6-ylthio)-9-(2-(1-(methylsulfonyl)piperidin-3-yl)ethyl)-9H-purin-6-amine; N-(2-((2-(8-((7-(1H-pyrazol-3-yl)-2,3-dihydrobenzo[b][1,4]dioxin-6-yl)thio)-6-amino-9H-purin-9-yl)ethyl)amino)ethyl) sulfamide; 8-((7-(1H-pyrazol-3-yl)-2,3-dihydrobenzo[b][1,4]dioxin-6-yl)thio)-9-(3-aminopropyl)-9H-purin-6-amine; 8-((7-(1H-pyrazol-3ryl)-2,3-dihydrobenzo[b][1,4]dioxin-6-yl)thio)-9-(3-(tert-butylamino)propyl)-9H-purm-6-amm\hat{ }9-(3-(isopropylamino)propyl)-8-((7-(5-methyl-1H-pyrazol-3-yl)-2,3-dihydrobenzo[b][1,4]dioxin-6-yl)thio)-9H-purin-6-amine; 8-((7-(5-methyl-1H-pyrazol-3-yl)-2,3-dihydrobenzo[b][1,4]dioxin-6-yl)thio)-9-(2-(neopentylamino)ethyl)-9H-purin-6-amine; 1-(8-((7-(1H-pyrazol-3-yl)-2,3-dihydrobenzo[b][1,4]dioxin-6-yl)thio)-6-amino-9H-purin-9-yl)-3-(isopropylamino)propan-2-ol; 5-(8-(7-(1H-pyrazol-3-yl)-2)3-dihydrobenzo[b][1,4]dioxin-6-ylthio)-6-amino-9H-purin-9-yl)pentane-1-sulfonamide; 6-(8-(7-(1H-pyrazol-3-yl)-2,3-dihydrobenzo[b][1,4]dioxin-6-ylthio)-6-amino-9H-purin-9-yl)hexanamide; 1-(3-(8-(7-(1H-pyrazol-3-yl)-2,3-dihydrobenzo[b3][1,4]dioxin-6-ylthio)-6-amino-9H-purin-9-yl)propyl)pyrrolidin-3-one; 8-((7-(1H-pyrazol-3-yl)-2,3-dihydrobenzo[b][1,4]dioxin-6-yl)methyl)-2-fluoro-9-(2-(isobutylarmno)ethyl)-9H-purin-6-amine; 1-(4-(2-(8-((7-(1H-pyrazol-3-yl)-2,3-dihydrobenzo[b][1,4]dioxin-6-yl)methyl)-6-amino-2-fluoro-9H-purin-9-yl)ethyl)piperidin-1-yl)ethanone; 1-(3-(2-(8-((7-(1H-pyrazol-3-yl)-2,3-dihydrobenzo[b][1,4]dioxin-6-yl)methyl)-6-amino-2-fluoro-9H-purin-9-yl)emyl)piperidin-1-yl)ethanone; 8-((7-(1H-pyrazol-3-yl)-2,3-dihydrobenzo[b][1,4]dioxin-6-yl)methyl)-2-fluoro-9-(2-(1-(methylsulfonyl)piperidin-3-yl)ethyl)-9H-purin-6-amine; 1-(3-(8-((7-(1H-pyrazol-3-yl)-2,3-dihydrobenzo[b][1,4]dioxin-6-yl)methyl)-6-amino-2-fluoro-9H-purin-9-yl)propyl)pyrrolidin-3-one; 8-((7-(1H-pyrazol-3-yl)-2,3-dihydrobenzo[b][1,4]dioxin-6-yl)methyl)-9-(3-(tert-butylamino)propyl)-2-fluoro-9H-purin-6-amine; 1-(8-((7-(1H-pyrazol-3-yl)-2,3-dihydrobenzo[b][1,4]dioxin-6-yl)methyl)-6-amino-2-fluoro-9H-purin-9-yl)-3-(tert-butylamino)propan-2-ol; 5-(8-((7-(1H-pyrazol-3-yl)-2,3-dihydrobenzo[b][1,4]dioxin-6-yl)methyl)-6-amino-2-fluoro-9H-purin-9-yl)pentane-1-sulfonamide; 6-(8-((7-(1H-pyrazol-3-yl)-2,3-dihydrobenzo[b][1,4]dioxin-6-yl)methyl)-6-amino-2-fluoro-9H-purin-9-yl)hexanamide; and 8-((7-(1H-pyrazol-3-yl)-2,3-dihydrobenzo[b][1,4]dioxin-6-yl)methyl)-9-(2-aminoethyl)-2-fluoro-9H-purin-6-amine.

In some embodiments of Formula VII, X₂ is a furan.

In some embodiments, the Hsp90 inhibitor is selected from the group consisting of: 8-((7-(furan-2-yl)-2,3-dihydrobenzo[b][1,4]dioxin-6-yl)thio)-9-(3-(isopropylamino)propyl)-9H-purin-6-amine; 9-(3-(isopropylamino)propyl)-8-((7-(5-methylflu-an-2-yl)-2,3-cUhydrobenzo[b][1,4]dioxin-6-yl)thio)-9H-purin-6-amine; 8-((7-(5-methylfuran-2-yl)-2,3-dihydrobenzo[b][1,4]dioxin-6-yl)thio)-9-(2-(neopentylamino)ethyl)-9H-purin-6-amine; 8-((7-(5-(ammomethyl)furan-2-yl)-2,3-dihydrobenzo[b][1,4]dioxin-6-yl)thio)-9-(2-(neopentylamino)ethyl)-9H-purin-6-amine; 8-(7-(5-methylfuran-2-yl)-2,3-dihydrobenzo[b][1,4]dioxin-6-ylthio)-9-(2-(l-(methylsulfonyl)piperidin-3-yl)ethyl)-9H-purin-6-amine; 1-(3-(2-(6-ammo-8-(7-(5-memylfuran-2-yl)-2,3-dihydrobenzo[b][1,4]dioxin-6-ylthio)-9H-purin-9-yl)ethyl)piperidin-1-yl)ethanone; 1-(4-(2-(6-amino-8-((7-(5-methylfuran-2-yl)-2,3-dihydrobenzo[b][1,4]dioxin-6-yl)thio)-9H-purin-9-yl)ethyl)piperidin-1-yl)ethanone; 1-(3-(2-(6-amino-8-(7-(5-(aminomethyl)furan-2-yl)-2,3-dihydrobenzo[b][1,4]dioxin-6-ylthio)-9H-purin-9-yl)ethyl)piperidin-1-yl)ethanone; 5-(6-amino-8-(7-(5-methylraran-2-yl)-2,3-dihydrobenzo[b][1,4]dioxin-6-ylthio)-9H-purin-9-yl)pentane-1-sulfonamide; 1-(3-(6-amino-8-(7-(5-methylfuran-2-yl)-2,3-dihydrobenzo[b][1,4]dioxin-6-ylthio)-9H-purin-9-yl)propyl)pyrrolidin-3-one; 1-(6-amino-8-((7-(5-methylfuran-2-yl)-2,3-dihydrobenzo[b][1,4]dioxin-6-yl)thio)-9H-purin-9-yl)-3-(isopropylamino)propan-2-ol; 9-(3-aminopropyl)-8-(7-(5-methylfuran-2-yl)-2,3-dihydrobenzo[b][1,4]dioxin-6-ylthio)-9H-purin-6-amine; N-(2-((2-(6-amino-8-((7-(furan-2-yl)-2,3-dihydrobenzo[b][1,4]dioxin-6-yl)tWo)-9H-purin-9-yl)ethyl)amino)emyl)sul& 3-((2-(6-amino-8-((7-(furan-2-yl)-2,3-dihydrobenzo[b][1,4]dioxin-6-yl)thio)-9H-purin-9-yl)ethyl)amino)-N-hydroxypropanamide; 9-(3-(tert-butylamino)propyl)-8-(7-(5-methylfuran-2-yl)-2,3-dihydrobenzo[b][1,4]dioxin-6-ylthio)-9H-purin-6-amine; 6-(6-amino-2-fluoro-8-((7-(5-methyloxazol-2-yl)-2,3-Hhydrobenzo[b][1,4]dioxin-6-yl)methyl)-9H-purin-9-yl)hexanamide; 2-fluoro-8-((7-(5-methylfuran-2-yl)-2,3-dihydrobenzo[b][1,4]dioxin-6-yl)methyl)-9-(2-(1-(methylsulfonyl)piperidin-3-yl)ethyl)-9H-purin-6-amine; 1-(3-(2-(6-amino-2-fluoro-8-((7-(5-methylfuran-2-yl)-2,3-dihydrobenzo[b][1,4]dioxin-6-yl)methyl)-9H-purin-9-yl)ethyl)piperidin-1-yl)ethanone; 1-(4-(2-(6-amino-2-fiuoro-8-((7-(5-methylfuran-2-yl)-2,3-dihydrobenzo[b][1,4]dioxin-6-yl)methyl)-9H-purin-9-yl)ethyl)piperidin-1-yl)ethanone; 1-(3-(2-(6-amino-8-((7-(5-(aminomethyl)furan-2-yl)-2,3-dihydrobenzo[b][1,4]dioxin-6-yl)methyl)-2-fluoro-9H-purin-9-yl)ethyl)piperidin-1-yl)ethanone; 2-fluoro-8-((7-(furan-2-yl)-2,3-dihydrobenzo[b][1,4]dioxin-6-yl)methyl)-9-(2-(isobutylamino)ethyl)-9H-purin-6-amine; 2-fluoro-9-(2-(isobutylamino)ethyl)-8-((7-(5-methylfuran-2-yl)-2,3-dihydrobenzo[b][1,4]dioxin-6-yl)methyl)-9H-purin-6-amine 8-((7-(5-(aminomethyl)ftiran-2-yl)-2,3-dihydrobenzo[b][1,4]dioxin-6-yl)methyl)-2-fluoro-9-(2-(isobutylamino)ethyl)-9H-purin-6-amine; 1-(3-(6-amino-2-fluoro-8-((7-(5-methyloxazol-2-yl)-2,3-dihydrobenzo[b][1,4]dioxin-6-yl)methyl)-9H-purin-9-yl)propyl)pyrrolidin-3-one; 2-chloro-8-((7-(5-methylfuran-2-yl)-2,3-dihydrobenzo[b][1,4]dioxin-6-yl)methyl)-9(methylsulfonyl)pyrrolidin-3-yl)ethyl)-9H-purin-6-amine; 9-(3-aminopropyl)-2-fluoro-8-((7-(5-methylfuran-2-yl)-2,3-dihydrobenzo[b][1,4]dioxin-6-yl)methyl)-9H-purin-6-amine; 5-(6-ammo-2-fluoro-8-((7-(5-methylfuran-2-yl)-2,3-dihydrobenzo[b][1,4]dioxin-6-yl)methyl)-9H purin-9-yl)pentane-1-sulfonamide; and 6-(6-amino-2-fluoro-8-((7-(5-methylfuran-2-yl)-2,3-dihydrobenzo[b][1,4]dioxin-6-yl)methyl)-9H-puiin-9-yl)hexanamide.

In some embodiments of Formula VII, X₂ is an oxazole.

In some embodiments, the Hsp90 inhibitor is selected from the group consisting of: 1-(3-(6-amino-8-(7-(oxazol-2-yl)-2,3-dihydrobenzo[b][1,4]dioxin-6-ylthio)-9H-purin-9-yl)propyl)pyrrolidin-3-one; 6-(6-amino-8-(7-(5-methyloxazol-2-yl)-2,3-dihydrobenzo[b][1,4]dioxin-6-ylthio)-9H-purin-9-yl)hexanamide; 8-(7-(5-methyloxazol-2-yl)-2,3-dmydrobenzo[b][1,4]dioxin-6-ylthio)-9-(2-(neopentylamino)ethyl)-9H-purin-6-amine; 1-(3-(2-(6-amino-8-(7-(5-methyloxazol-2-yl)-2,3-dihydrobenzo[b][1,4]dioxin-6-ylthio)-9H-purin-9-yl)ethyl)piperidin-1-yl)ethanone; 1-(4-(2-(6-amino-8-((7-(5-methyloxazol-2-yl)-2,3-dihydrobenzo[b][1,4]dioxin-6-yl)thio)-9H-purin-9-yl)ethyl)piperi 1-yl)ethanone; 8-((7-(5-methyloxazol-2-yl)-2,3-dihydrobenzo[b][1,4]dioxin-6-yl)thio)-9-(2-(1-(methylsulfonyl)piperidin-3-yl)ethyl)-9H-purin-6-amine; 5-(6-amino-8-(7-(5-methyloxazol-2-yl)-2,3-dihydrobenzo[b][1,4]dioxin-6-ylthio)-9H-purin-9-yl)pentane-1-sulfonamide; N-(3-(6-amino-8-((7-(5-methyloxazol-2-yl)-2,3-dihydrobenzo[b][1,4]dioxin-6-yl)thio)-9H-purin-9-yl)propyl)methanesulfonamide; 1-(2-(4-(6-amino-8-(7-(5-methyloxazol-2-yl)-2,3-dihydrobenzo[b][1,4]dioxin-6-ylthio)-9H-purin-9-yl)butyl)pyrrolidin-1-yl)ethanone; 1-(6-amino-8-((7-(5-methyloxazol-2-yl)-2,3-dihydrobenzo[b][1,4]dioxin-6-yl)thio)-9H-purin-9-yl)-3-(isopropylamino)propan-2-ol; 9-(3-(tert-butylamino)propyl)-8-((7-(oxazol-2-yl)-2,3-dihydrobenzo[b][1,4]dioxin-6-yl)thio)-9H-purin-6-amine; 9-(3-aminopropyl)-8-((7-(oxazol-2-yl)-2,3-dihydrobenzol¾][1,4]dioxin-6-yl)thio)-9H-purin-6-amine; 8-((7-(furan-2-yl)-2,3-dihydrobenzo[b][1,4]dioxin-6-yl)thio)-9-(2-(isobutylamino)ethyl)-9H-purin-6-amine; 9-(3-(isopropylamino)propyl)-8-((7-(oxazol-2-yl)-2,3-dihydrobenzo[b][1,4]dioxin-6-yl)thio)-9H-purin-6-amine; 1-(2-(4-(6-amino-8-(7-(5-methyloxazol-2-yl)-2,3-dihy<kobenzo[b][1,4]dioxin-6-yltWo)-9H-purin-9-yl)butyl)pyrrolidm 1-yl)ethanone; 1-(4-(2-(6-amino-8-((7-(5-methyloxazol-2-yl)-2,3-dihydrobenzo[b][1,4]dioxin-6-yl)thio)-9H-purin-9-yl)ethyl)piperidin-1-yl)ethanone; 8-((7-(5-methyloxazol-2-yl)-2,3-dihydrobenzo[b][1,4]dioxin-6-yl)thio)-9-(2-(1-(methylsulfonyl)piperidin-3-yl)ethyl)-9H-purin-6-amine; 2-fluoro-9-(3-(isopropylamino)propyl)-8-((7-(oxazol-2-yl)-2,3-dihydrobenzo[b][1,4]dioxin-6-yl)methyl)-9H-purin-6-amine; 2-fluoro-9-(3-(isopropylamino)propyl)-8-((7-(5-methyloxazol-2-yl)-2,3-dihydrobenzo[b][1,4]dioxin-6-yl)methyl)-9H-purin-6-amine; 9-(3-(tert-butylamino)propyl)-2-fluoro-8-((7-(oxazol-2-yl)-2,3-dihydrobenzo[b][1,4]dioxin-6-yl)methyl)-9H-purin-6-amine; 9-(3-(tert-butylamino)propyl)-2-fluoro-8-((7-(5-methyloxazol-2-yl)-2,3-dihydrobenzo[b][1,4]dioxin-6-yl)meth.yl)-9H-purin-6-amine; 6-(6-amino-2-fluoro-8-((7-(5-methyloxazol-2-yl)-2,3-dihydrobenzo[b][1,4]dioxin-6-yl)methyl)-9H-purin-9-yl)hexanamid\hat{ }5-(6-amino-2-fluoro-8-((7-(5-methyloxazol-2-yl)-2,3-dihydrobenzo[b][1,4]dioxin-6-yl)methyl)-9H-purin-9-yl)pentane-1-sulfonamide; 1-(3-(6-amino-2-fluoro-8-((7-(5-methyloxazol-2-yl)-2,3-dihydrobenzo[b][1,4]dioxin-6-yl)methyl)-9H-piirin-9-yl)propyl)pyrrolidin-3-one; 1-(3-(6-amino-2-fluoro-8-((7-(oxazol-2-yl)-2,3-dihydrobenzo[b][1,4]dioxin-6-yl)methyl)-9H-purin-9-yl)propyl)pyTrolidin-3-one; and 9-(3-aminopropyl)-2-fluoro-8-((7-(5-metl yloxazol-2-yl)-2,3-dihydrobenzo[b][1,4]dioxin-6-yl)methyl)-9H-purin-6-amine.

In some embodiments of Formula VII, X₂ is alkynyl.

In some embodiments, the Hsp90 inhibitor is selected from the group consisting of: 8-((7-ethynyl-2,3-dihydrobenzo[b][1,4]dioxin-6-yl)thio)-9-(3-(isopropylamino)propyl)-9H-purin-6-amine; 3-(3-(6-amino-8-(7-ethynyl-2,3-dihydrobenzo[b][1,4]dioxin-6-ylthio)-9H-purin-9-yl)propyl)pyrrolidine-1-carbaldehyde; 8-((7-ethynyl-2,3-dihydrobenzo[b][1,4]dioxin-6-yl)thio)-9-(2-(neopentylamino)ethyl)-9H-purin-6-amine; 9-(2-aminoethyl)-8-((7-ethynyl-2,3-dihydrobenzo[b][1,4]dioxin-6-yl)thio)-9H-purin-6-amine; 1-(3-(2-(6-amino-8-(7-ethynyl-2,3-dihydrobenzo[b][1,4]dioxin-6-ylthio)-9H-purin-9-yl)ethyl)piperidin-1-yl)ethanone; 8-(7-ethynyl-2,3-dihydrobenzo[b][1,4]dioxin-6-ylthio)-9-(2-(1-(methylsulfonyl)piperidin-3-yl)ethyl)-9H-purin-6-amine; N-(2-((2-(6-amino-8-((7-ethynyl-2,3-dihydrobenzo[b][1,4]dioxin-6-yl)thio)-9H-purin-9-yl)ethyl)amino)ethyl)sulfamide; 9-(3-aminopropyl)-8-((7-ethynyl-2,3-dihydrobenzo[b][1,4]dioxin-6-yl)thio)-9H-purin-6-amine; 6-(6-amino-8-(7-ethynyl-2,3-dihydrobenzo[b][1,4]dioxin-6-ylthio)-9H-purin-9-yl)hexanamide; 5-(6-amino-8-(7-ethynyl-2,3-dihydrobenzo[b][1,4]dioxin-6-ylthio)-9H-purin-9-yl)pentane-1-sulfonamide; 1-(6-amino-8-((7-ethynyl-2,3-dihydrobenzo[b][1,4]dioxin-6-yl)thio)-9H-purin-9-yl)-3-(isopropylamino)propan-2-ol; 9-(3-(tert-butylamino)propyl)-8-(7-ethynyl-2,3-dihydrobenzo[b][1,4]dioxin-6-ylthio)-9H-purin-6-amine; 8-(7-ethynyl-2,3-dihydrobenzo[b]i1,4]dioxin-6-ylthio)-9-(2-(1-methylpiperidin-2-yl)ethyl)-9H-purin-6-amine; 8-(7-ethynyl-2,3-dihydrobenzo[b][1,4]dioxin-6-ylthio)-9-(2-(1-methylpiperidin-3-yl)ethyl)-9H-purin-6-amine; 9-(2-aminoethyl)-8-(7-ethynyl-2,3-dihydrobenzo[b][1,4]dioxin-6-ylthio)-9H-purin-6-amine; 8-((7-ethynyl-2,3-dihydrobenzo[b][1,4]dioxin-6-yl)methyl)-2-fluoro-9-(2-(isobutyl amino)ethyl)-9H-purin-6-amine; 8-((7-ethynyl-2,3-dihydrobenzo[b][1,4]dioxin-6-yl)methyl)-2-fluoro-9-(2-(1-(methylsulfonyl)piperidin-3-yl)ethyl)-9H-purin-6-amine; 1-(3-(2-(6-amino-8-((7-ethynyl-2,3-dihydrobenzo[b][1,4]dioxin-6-yl)methyl)-2-fluoro-9H-purin-9-yl)ethyl)piperidin-1-yl)ethanone; 3-(2-(6-amino-8-((7-ethynyl-2,3-dihydrobenzo[b][1,4]dioxin-6-yl)memyl)-2-fluoro-9H-purin-9-yl)ethyI)piperidine-1-carbaldchyde; 1-(3-(6-amino-8-((7-ethynyl-2,3-dihydrobenzo[b][1,4]dioxin-6-yl)methyl)-2-fluoro-9H-purin-9-yl)propyl)pyrrolidin-3-one; 6-(6-amino-8-((7-ethynyl-2,3-dihydrobenzo[b][1,4]dioxin-6-yl)methyl)-2-fluoro-9H-purin-9-yl)hexanamide; 1-(6-amino-8-((7-ethynyl-2,3-dihydrobenzo[b][1,4]dioxin-6-yl)methyl)-2-flaoro-9H-purin-9-yl)-3-(tert\hat{ } butylamino)propan-2-ol; 5-(6-amino-8-((7-ethynyl-2,3-dihydrobenzo[b][1,4]dioxin-6-yl)methyl)-2-fluoro-9H-purin-9-yl)pentane-1-sulfonamide; 8-((7-ethynyl-2,3-dihydrobenzo[b][1,4]dioxm-6-yl)methyl)-2-fl\hat{ }\hat{ }amine; 9-(3-(tert-butylamino)propyl)-8-((7-ethynyl-2,3-dihydrobenzo[b][1,4]dioxin-6-yl)methyl)-2-fluoro-9H-purin-6-amine; 9-(3-aminopropyl)-8-((7-ethynyl-2,3-dihydrobenzo[b][1,4]dioxin-6-yl)methyl)-2-fluoro-9H-purin-6-amine; 8-((7-ethynyl-2,3-dihydrobenzo[b][1,4]dioxin-6-yl)methyl)-2-fluoro-9-(2-(1-methylpiperidin-2-yl)ethyl)-9H-purin-6-amine; and 8-((7-ethynyl-2,3-dihydrobenzo[b][1,4]dioxin-6-yl)methyl)-2-fluoro-9-(2-(1-methylpiperidin-3-yl)ethyl)-9H-purin-6-amine

Another class of Hsp90 inhibitors of this disclosure have the general structure of Formula VIII:

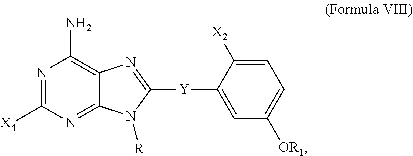

wherein

(a) R₁ is alkyl;

(b) Y is S or CH₂,

(c) X4 is H or halogen,

(d) X₂ is a saturated or unsaturated non-aromatic carbocycle or heterocycle, an aryl, an alkylamino, a dialkylamino, an alkynyl or is part of a ring formed by R; and

(e) R is hydrogen, alkyl, alkenyl, or alkynyl, linear, branched or cyclic, optionally including heteroatoms such as N, S or O, optionally connected to the 2′-position to form an 8 to 10 member ring.

Other classes of Hsp90 inhibitors of this disclosure have the general structure of Formula IX, X or XI:

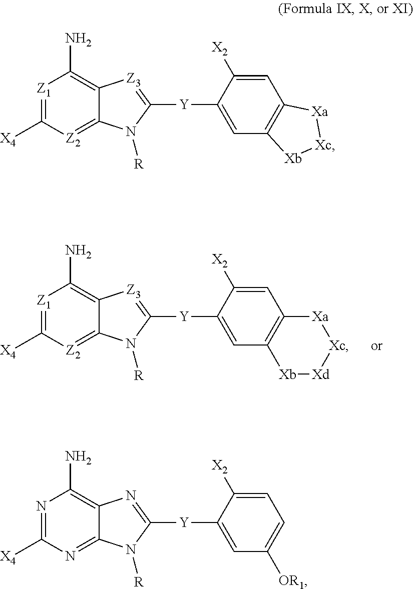

wherein

(a) Y is CH₂, S, O, C=0, C═S, or N;

(b) Xd is H or halogen;

(c) Xa, Xb, Xc and Xd are independently selected from C, O, N, S, carbonyl, and thionyl, connected by single or double bonds with H as needed to satisfy valence,

(d) X₂ is an alkynyl group and

(e) R is a group listed in Table A.

Other classes of Hsp90 inhibitors of this disclosure have the general structure of Formula XII, XIII or XIV:

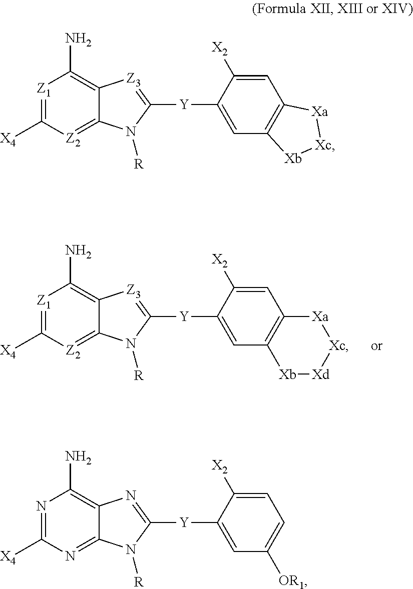

wherein

(a) Y is CH2, S, 0, C=0, OS, or N; (b) X4 is H or halogen;

(c) Xa, Xb, Xc and Xd are independently selected from C, O, N, S, carbonyl, and thionyl, connected by single or double bonds with H as needed to satisfy valence,

(d) X₂ is a furan, thiophene, pyrazole, oxazole or thiazole and

(e) R is a group listed in Table A.

### Table A: R Groups for Formulae VI-XIV

1. R is hydrogen, a C₁ to C₁₀ Alkyl, alkenyl, alkynyl, or an alkoxyalkyl group, optionally including heteroatoms such as N or O, or a targeting moiety connected to N9 via a linker,
2. R is hydrogen, straight- or branched-, substituted or unsubstituted alkyl, substituted or unsubstituted alkenyl, substituted or unsubstituted alkynyl, in which one or more methylenes can be interrupted or terminated by O, S, S(O), S0₂, N(R₂₁₈), C(0), substituted or unsubstituted aryl, substituted or unsubstituted heteroaryl, substituted or unsubstituted heterocyclic; substituted or unsubstituted cycloalkyl; or

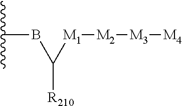

B is a linker; R₂₁₀ is selected from the group consisting of hydrogen, N(R₂)COR₄, N(R₂CON(R₃)R₄, N(R₂)COOR₄, M(R₂S(0n)R₃, N(R₂)S(0)nN(R₃)R₄; where R₂ and R₃ are independently selected from hydrogen, aliphatic or substituted aliphatic; R₄ is selected from the group consisting of: aryl, substituted aryl, heteroaryl, substituted heteroaryl, heterocyclic, substituted heterocyclic, cycloalkyl, substituted cycloalkyl, cycloalkenyl, substituted cycloalkenyl, and substituted or unsubstituted -Ci-C₆ alkyl, —C₂-C₆ alkenyl, or —C₂-C₆alkynyl each containing 0, 1, 2, or 3 heteroatoms selected from O, S or N; n is 1 or 2; Mi is absent or selected from substituted or unsubstituted -Ci-C₆ alkyl, —C₂-C₆alkenyl, or —C₂-C₆ alkynyl, aryl, substituted aryl heteroaryl, substituted heteroaryl;
M2 is absent, O, S, SO, S0₂, N(R₂) or CO;
M3 is absent, O, S, SO, SO, N(R₂), CO, Ci-C₆ alkyl, C₂-C₆alkenyl, C₂-C₆ alkynyl, cycloalkyl, heterocyclic, aryl, or heteroaryl;
M4 is hydrogen, NR₅R₆, CF₃, OR₄, halogen, substituted or unsubstituted —C₁C₆ alkyl, —C₂-C₆ alkenyl, or —C₂-C₆ alkynyl, cycloalkyl, substituted cycloalkyl, heterocyclic, substituted heterocyclic, aryl, substituted aryl, heteroaryl or substituted heteroaryl; where R₅ and R₆ are independently selected from the group consisting of hydrogen, aliphatic, substituted aliphatic, aryl, substituted aryl, heteroaryl, substituted heteroaryl, heterocyclic, substituted heterocyclic, cycloalkyl or substituted cycloalkyl; provided that —R and -Mi-M₂-M₃-M₄ cannot be both hydrogen.

### 3. R is

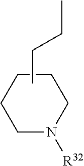

wherein R³² is
(a) hydro;
(b) C₁-C₆ alkyl optionally substituted with 1, 2, 3, 4, or 5 substituents each independently chosen from the group of halo, hydroxyl, amino, cyano, and —C(=0)R³¹ wherein R³¹ is amino;
(c) —C(=Q)R³³, wherein R³³ is selected from the group consisting of:
(1) hydro,
(2) C₁C₁₀ (e.g., C₁-C₆) alkyl optionally substituted with 1, 2, 3, 4, or 5 substituents each independently chosen from the group of (A) halo, (B) hydroxyl, (C) thiol, (D) cyano, (E) C₁-C₆ haloalkyl (e.g., trifluoromethyl), (F) C₁-C₆ alkoxy (e.g., methoxy) optionally substituted with C₁-C₆ alkoxy (e.g., methoxy), (G)C-amido, (H)N-amido, (I) sulfonyl, (J) —N(R²²)(R²³) wherein R²² and R²³ are independently hydro, C₁C₆ alkyl, sulfonyl, and C-carboxy,
(3) C₁-C₆ cycloalkyl optionally substituted with 1, 2, 3, 4, or 5 substituents each independently chosen from the group of halo, hydroxyl, amino, cyano, and C₁-C₆ haloalkyl (e.g., trifluoromethyl), and
(4) C₁-C₆ alkoxy optionally substituted with 1, 2, 3, 4, or 5 substituents each independently chosen from halo, hydroxyl, amino, cyano, and C₁-C₆ haloalkyl (e.g., trifluoromethyl),
(f) heterocycle or heterocyclylalkyl, optionally substituted with 1, 2, 3, 4, or 5 substituents independently chosen from halo, hydroxyl, amino, cyano, trihalomethyl, and C₁-C₄ alkyl optionally substituted with 1, 2, 3, or 4 substituents independently chosen from halo, hydroxyl, amino, cyano, C₁-C₆ haloalkyl (e.g., trifluoromethyl) (e.g., tetrazole-5-yl optionally substituted with 1, 2, 3, or 4 C₁-C₄ alkyl);
(g) sulfonyl; and
(h) optionally substituted heteroaryl
4. R is —R⁵⁴—R⁵, wherein
R⁵⁴ is —(CH₂)n- wherein n=0-3, —C(0), —C(S), —S0₂-, or —S0₂N—; and
R⁵⁵ is alkyl, aromatic, heteroaromatic, alicyclic, or heterocyclic, each of which is optionally bi- or tri-cyclic, and optionally substituted with H, halogen, lower alkyl, lower alkenyl, lower alkynyl, lower aryl, lower alicyclic, aralkyl, aryloxyalkyl, alkoxyalkyl, perhaloalkyl, perhaloalkyloxy, perhaloacyl, —N₃, —SR⁵⁸, —OR⁵⁸, —CN, —C0₂R⁵⁹, —N0₂, or —NR⁵⁸R⁵^{₁}⁰,
R⁵⁸ is hydrogen, lower alkyl, lower aryl, or —C(O) R5′5;
R⁵⁹ is lower alkyl, lower aryl, lower heteroaryl, or —NR⁵^{₁}⁰R⁵^{₁}⁰; and
R⁵^{₁}⁰ is independently hydrogen or lower alkyl
5. R is selected from the group consisting of H, optionally substituted alkyl, optionally substituted alkenyl, optionally substituted alkynyl, optionally substituted aryl, optionally substituted alicyclic, optionally substituted araalkyl, optionally substituted aryloxyalkyl, optionally substituted alkoxyalkyl, alkylaminoalkyl, alkylcarbonylaminoalkyl, alkylcarbonyoxylalkyl, optionally substituted heterocyclic, hydroxyalkyl, haloalkyl, and perhaloalkyl.
6. R is H, SR₇₁, SOR₇₁, S0₂R₇₁, OR₇₁, COOR₇₁, CONR₇₁R₇₂, —CN, C₁₋₆ alkyl, C₂₋₆ alkenyl, C₂₋₆ alkynyl, —R₇AOR₇B—R₇AR₇B, —R₇ANR₇₁R₇B, —R₇ASR₇B, —R₇ASOR₇B or —R₇AS0₂R₇B, cycloalkyl, heteroalkyl, heterocycloalkyl, aryl, heteroaryl, alkylaryl, arylalkyl, alkylheteroaryl, heteroarylalkyl, NR₇₁R₇₂, —OSO₂N(R₇C₂, —N(R₇C)SO₂OH, —N(R₇C)SO₂R₇C, —R₇AOSO₂N(R₇C)2, or —R₇AN(R₇C)OSO₂R₇C; R₇₁ and R₇₂ are independently selected from the group consisting of H, COOR₇B, CON(R₇C)₂C₁₋₆ alkyl, C₂₋₆ alkenyl, C₂₋₆ alkynyl, —R₇AOR₇B˜, —R₇ANR₇B, —R₇ANR₇₁R₇B, —R₇ASR₇B, —R₇ASQR₇B or —R₇ASO₂R₇B cycloalkyl, heteroalkyl, heterocycloalkyl, aryl, heteroaryl, alkylaryl, arylalkyl, alkylheteroaryl, and heteroarylalkyl; each R₇A is independently C₁₋₆ alkyl, C₂₋₆ alkenyl, C₂₋₆ alkynyl, cycloalkyl, heteroalkyl, heterocycloalkyl, aryl, heteroaryl, alkylaryl, arylalkyl, alkylheteroaryl, alkylheteroarylalkyl, or heteroarylalkyl; and
each R₇B is independently H, C₁₋₆ alkyl, C₂₋₆ aLkenyl, C₂₋₆ alkynyl, cycloalkyl, heteroalkyl, heterocycloalkyl, aryl, heteroaryl, alkylaryl, arylalkyl, alkylheteroaryl, heteroarylalkyl, —SO₂OH—SO₂N(R₇A)₂, —SO₂NHR₇A or —SO₂NH₂; and each R.sub.C is independently H, C₁₋₆ alkyl, C₂₋₆ alkenyl, C₂₋₆ alkynyl, cycloalkyl, heteroalkyl, heterocycloalkyl, aryl, heteroaryl, alkylaryl, arylalkyl, alkylheteroaryl, or heteroarylalkyl;
7A. R is hydrogen, straight- or branched-, substituted or unsubstituted alkyl, substituted or unsubstituted alkenyl, substituted or unsubstituted alkynyl, which one or more methylenes can be interrupted or terminated by O, S, S(O), SO₂, N(R₈₈), C(O), substituted or unsubstituted aryl, substituted or unsubstituted heteroaryl, substituted or unsubstituted heterocyclic; substituted or unsubstituted cycloalkyl; where R₈₈ is hydrogen, acyl, aliphatic or substituted aliphatic,
7B. R is -M1-M2-M3-M4, wherein
M₁ is absent, C₁-C₆ alkyl, C₂-C₆ alkenyl, C₂-C₆ alkynyl, aryl or heteroaryl;
M₂ is absent, O, S, SO, SO₂, N(R₈₈), or C=0;
M₃ is absent, C=0, O, S, SO, SO₂ or N(R₈₈); and
M₄ is hydrogen, halogen, CN, N₃, hydroxy, substituted hydroxy, amino, substituted amino, CF₃, C₁-C₆ alkyl, C₂-C₆ alkenyl, C₂-C₆ alkynyl, cycloalkyl, heterocyclic, aryl or heteroaryl.

“Alkyl” (or alkyl group) refers to a linear, cyclic or branched saturated hydrocarbon, for example a hydrocarbon having from 1 to 10 carbon atoms, in which the atom directly attached to the central structure is a carbon atom. Such an alkyl group may include substituents other than hydrogen, for example an oxygen-containing group including without limitation hydroxyl and alkoxy; a halogen group; a nitrogen-containing group including without limitation amino, amido and alkylamino; an aryl group; a sulfur-containing group including without limitation thioalkyl; and/or a non-aromatic cyclic group including heterocycles and carbocycles. Carbon atoms in these substituents may increase the total number of carbon atoms in the alkyl group to above 10 without departing from the spirit of this disclosure. All references to alkyl groups in the specification and claims hereof encompass both substituted and unsubstituted alkyl groups unless the context is clearly to the contrary.

“Alkenyl” (or akenyl group) refers to a linear, cyclic or branched hydrocarbon, for example a hydrocarbon having from 1 to 10 carbon atoms, and at least one double bond, in which the atom directly attached to the central structure is a carbon atom. The alkenyl group may include any of the substituents mentioned above for an alkyl group. All references to alkenyl groups in the specification and claims hereof encompass both substituted and unsubstituted alkenyl groups unless the context is clearly to the contrary.

“Alkynyl” (or alkynyl group) refers to a linear, cyclic or branched hydrocarbon, for example a hydrocarbon having from 1 to 10 carbon atoms, and at least one triple bond, in which the atom directly attached to the central structure is a carbon atom. The alkynyl group may include any of the substituents mentioned above for an alkyl group. All references to alkynyl groups in the specification and claims hereof encompass both substituted and unsubstituted alkynyl groups unless the context is clearly to the contrary.

“Aryl” (or aryl group) refers to any group derived from a simple aromatic ring. Aryl group includes heteroaryl. Aryl groups may be substituted or unsubstituted. When X2, X4 and R is identified as an aryl group (particularly for Formulae VI-XIV), an atom of the aryl ring is bound directly to an atom of the central structure. An aryloxy substituent is an aryl group connected to the central structure through an oxygen atom. The aryl group may include any of the substituents mentioned above for an alkyl group, and in addition an aryl group may include an alkyl, alkenyl or alkynyl group. All references to aryl groups in the specification and claims hereof encompass both substituted and unsubstituted aryl groups unless the context is clearly to the contrary.

“Amino” (or amino group) refers to any group which consists of a nitrogen attached by single bonds to carbon or hydrogen atoms. In certain instances, the nitrogen of the amino group is directly bound to the central structure. In other instances, an amino group may be a substituent on or within a group, with the nitrogen of the amino group being attached to the central structure through one or more intervening atoms. Examples of amino groups include NH2, alkylamino, alkenylamino groups and N-containing non-aromatic heterocyclic moiety (i.e., cyclic amines). Amino groups may be substituted or unsubstituted. All references to amino groups in the specification and claims hereof encompass substituted and unsubstituted amino groups unless the context is clearly to the contrary.

“Halogen” (or halogen group) refers to fluorine, chlorine, bromine or iodine.

“Heterocyclic” (or heterocyclic group) refers to a moiety containing at least one atom of carbon, and at least one atom of an element other than carbon, such as sulfur, oxygen or nitrogen within a ring structure. These heterocyclic groups may be either aromatic rings or saturated and unsaturated non-aromatic rings. Heterocylic groups may be substituted or unsubstituted. All references to heterocyclic groups in the specification and claims encompass substituted and unsubstituted heterocyclic groups unless the context is clearly to the contrary.

In the compounds provided herein, all of the atoms have sufficient hydrogen or non-hydrogen substituents to satisfy valence, or the compound includes a pharmaceutically acceptable counterion, for example in the case of a quaternary amine.

The various oral formulations provided herein may comprise one or more of any of the foregoing Hsp90 inhibitors. In some embodiments, the active compound (or API, as the terms are used interchangeably herein) is Compound 1 or Compound 1a. In some embodiments, the active compound is Compound 2 or Compound 2a. These active compounds may be provided as free base forms, such as but not limited to the free base form of Compound 2. These active compounds may be provided as hydrochloride or dihydrochloride forms such as but not limited to Compound 1 2HCl or Compound 2 2HCl. Other salt forms are contemplated including maleate, malate, oxalate and nitrate salts of the Hsp90 inhibitors provided herein including but not limited to Compound 1, Compound 1a, Compound 2, and Compound 2a. These and other salts forms are discussed below in greater detail.

Additional examples of compounds of this type are provided by in US published application US 2009/0298857 A1 and in U.S. Pat. No. 7,834,181, the entire disclosures of which as they relate to such Hsp90 inhibitors and classes thereof are incorporated by reference herein.

Reference can also be made to PCT Publication No. WO2011/044394 (Application No. PCT/US2010/051872) for additional compounds that can be used as Hsp90 inhibitors and that are contemplated as part of this disclosure. The teachings of such reference are incorporated by reference herein, particularly with respect to their disclosure of compounds of any one of Formulae VI-XIV (as named herein).

The Hsp90 inhibitors may be provided as pharmaceutically acceptable salts. The term “pharmaceutically acceptable salt” refers to those salts which retain the biological effectiveness and properties of the “free” compounds provided herein. A pharmaceutically acceptable salt can be obtained from the reaction of the free base of an active compound provided herein with an inorganic acid, for example, hydrochloric acid, hydrobromic acid, sulfuric acid, nitric acid, phosphoric acid, and the like, or an organic acid, for example, sulfonic acid, carboxylic acid, organic phosphoric acid, methanesulfonic acid, ethanesulfonic acid, p-toluenesulfonic acid, citric acid, fumaric acid, maleic acid, succinic acid, benzoic acid, salicylic acid, lactic acid, tartaric acid (e.g., (+)-tartaric acid or (−)-tartaric acid or mixtures thereof), and the like. Additional non-limiting examples of suitable acids include acetic acid, acetylsalicylic acid, adipic acid, alginic acid, ascorbic acid, aspartic acid, benzenesulfonic acid, bisulfic acid, boric acid, butyric acid, camphoric acid, camphorsulfonic acid, carbonic acid, citric acid, cyclopentanepropionic acid, digluconic acid, dodecylsulfic acid, formic acid, glyceric acid, glycerophosphoric acid, glycine, glucoheptanoic acid, gluconic acid, glutamic acid, glutaric acid, glycolic acid, hemisulfic acid, heptanoic acid, hexanoic acid, hippuric acid, hydroiodic acid, hydroxyethanesulfonic acid, malic acid, malonic acid, mandelic acid, mucic acid, naphthylanesulfonic acid, naphthylic acid, nicotinic acid, nitrous acid, oxalic acid, pelargonic, propionic acid, saccharin, sorbic acid, thiocyanic acid, thioglycolic acid, thiosulfuric acid, tosylic acid, undecylenic acid, and naturally and synthetically derived amino acids.

Certain active compounds provided herein have acidic substituents and can exist as pharmaceutically acceptable salts with pharmaceutically acceptable bases. The present disclosure includes such salts. Examples of such salts include metal counterion salts, such as sodium, potassium, lithium, magnesium, calcium, iron, copper, zinc, silver, or aluminum salts, and organic amine salts, such as methylamine, dimethylamine, trimethylamine, diethylamine, triethylamine, n-propylamine, 2-propylamine, or dimethylisopropylamine salts, and the like.

The term “pharmaceutically acceptable salt” includes mono-salts and compounds in which a plurality of salts is present, e.g., di-salts and/or tri-salts. Pharmaceutically acceptable salts can be prepared by methods known to those in the art.

### Excipients Generally

Excipients are compounds included in a manufacturing process or in a final formulation other than the active pharmaceutical ingredient (API). Excipients may be included in a manufacturing process or in a final formulation for the purpose of improving stability (e.g., long-term stabilization), bulking up solid formulations (and referred to interchangeably as bulking agents, fillers, diluents), reducing viscosity (for liquid formulations), enhancing solubility, improving flowability or non-stick properties, and/or improving granulation.

Excipients are generally regarded as inactive because when administered in the absence of the API they have no therapeutic effect. However, they may confer a therapeutic enhancement on the API in the final formulation for example by facilitating API absorption, reducing viscosity, enhancing solubility, improving bioavailability, long-term stability, and the like, and in that sense, they can improve the therapeutic efficacy of the API.

When used in the manufacturing process, excipients can aid in the handling of the API such as by facilitating powder flowability or non-stick properties, in addition to aiding in vitro stability such as preventing denaturation or aggregation over the expected shelf life.

The selection of appropriate excipients also depends upon the route of administration and the dosage form, as well as the API and other factors.

Notwithstanding the foregoing, all excipients are pharmaceutically acceptable intending that each is compatible with the other excipients and ingredients of a pharmaceutical formulation, and suitable for use in contact with the tissue or an organ of a patient without excessive toxicity, irritation, allergic response, immunogenicity, or other problems or complications, commensurate with a reasonable benefit/risk ratio.

Pharmaceutically acceptable excipients are known in the art; see, e.g., Pharmaceutical Preformulation and Formulation (Gibson, ed., 2nd Ed., CRC Press, Boca Raton, Fla., 2009); Handbook of Pharmaceutical Additives (Ash and Ash, eds., 3rd Ed., Gower Publishing Co., Aldershot, U K, 2007); Remington's Pharmaceutical Sciences (Gennaro, ed., 19th Ed., Mack Publishing, Easton, Pa., 1995); and Handbook of Pharmaceutical Excipients (Amer. Pharmaceutical Ass'n, Washington, D C, 1986).

A variety of excipients, their intended purpose, and examples of each are provided below. Certain compounds have two or more functions, as will be clear from this list.

Anti-adherents are compounds that reduce adhesion of a powder or granulation to manufacturing device surfaces such as but not limited to tablet press surfaces (e.g., punch faces or die walls). Examples of anti-adherents include magnesium stearate, talc and starch. Anti-adherents may also be referred to as anti-tack agents or flow aids.

Binders are compounds that bind (or hold) together components of a solid form such as a tablet. They may also function to provide mechanical strength to a solid form such as a tablet. Examples of binders include saccharides and saccharide derivatives such as disaccharides (e.g., sucrose and lactose); polysaccharides and polysaccharide derivatives (e.g., starches, cellulose and modified cellulose such as microcrystalline cellulose and cellulose ethers such as hydroxypropyl cellulose (HPC); and sugar alcohols such as xylitol, sorbitol or maltitol; proteins such as gelatin; and synthetic polymers such as polyvinylpyrrolidone (PVP), polyethylene glycol (PEG).

Fillers are compounds that add bulk, and thus mass, to the formulation, such as a low dose formulation. Examples of fillers/diluents include but are not limited to gelatin, cellulose, gum tragacanth, Pearlitol 300DC, sucrose, Prosolv HD90, lactose, and F-Melt. Certain compounds can function as both fillers and binders.

Lubricants are compounds that reduce friction, as may occur for example in blending, roller compaction, tablet manufacture (e.g., during ejection of tablets between the walls of tablet and the die cavity), and capsule filling. Lubricants are also used to increase the flowability of a solid such as a powder. They may accomplish this by reducing stickiness or clumping of components to each other or to mechanical devices or surfaces such as tablet presses and capsule filling devices. Examples of lubricants include but are not limited to metallic salts of fatty acids such as magnesium stearate, zinc stearate, and calcium stearate, silicon dioxide, fatty acids such as stearic acid and its salts and derivatives, palmitic acid and myristic acid, fatty acid esters such as glyceride esters (glyceryl monostearate, glyceryl tribehenate, and glyceryl dibehenate), sugar esters (sorbitan monostearate and sucrose monopalmitate), inorganic materials such as talc (a hydrated magnesium silicate (Mg₃Si₄O₁₀(OH)₂)), silica, PRUV®, and Lubripharm. Depending on the particular species, certain lubricants can also act as anti-adherents such as flow aids or anti-tack agents, and/or as glidants. One commercially available form of sodium stearyl fumarate is PRUV®. It may be used as a tablet lubricant when other lubricants present formulation and/or manufacturing challenges. PRUV® may offer the following advantages: high degree of API compatibility, robustness to over-lubrication, no adverse effect on bioavailability, and improved appearance of effervescent solutions.

Glidants are compounds that are added to solid forms such as powders and granulations to improve their flowability. They may accomplish this by reducing particle friction and adhesion. They may be used in combination with lubricants. Examples of glidants include but are not limited to magnesium carbonate, magnesium stearate, fumed silica (e.g., colloidal silicon dioxide) (for example at about 0.25-3% concentration), starch, and talc (for example at about 5% concentration).

Distintegrating agents (also referred to herein as disintegrants) are compounds that expand and dissolve when wet, thereby causing the solid form to break apart upon contact with fluid in the digestive tract. Disintegrants may be used to avoiding clumping in the stomach, etc. Examples of disintegrating agents include but are not limited to crosslinked polymers such as crosslinked polyvinylpyrrolidone (crospovidone), alginate, Primogel, corn starch, a sugar alcohol (e.g., mannitol, sorbitol, maltitol, and xylitol), a cellulose derivative (e.g., methylcellulose, cross-linked carboxymethyl cellulose, cross-linked sodium carboxymethyl cellulose (croscarmellose sodium), low substituted hydroxypropylcellulose, microcrystalline cellulose), cross-linked derivatives of starch, and pregelatinized starch.

Dispersion agents are compounds that deflocculate solids and thus reduce the viscosity of a dispersion or paste. A solid material dispersed in a liquid requires an additive to make the dispersion process easier and more stable. A dispersing agent or dispersant plays such as role. Because of this effect, solid loading (i.e., the amount of dispersible powdered material) can be increased. The dispersion phase can be time- and energy-consuming due to the different surface tensions of the liquids (e.g., resin, solvents) and the solids (e.g., fillers, additives). Therefore, a dispersion agent is used to produce stable formulations and ensure storage stability (e.g., no viscosity instability, no separation, etc.). Example of a dispersion agent include calcium silicate and docusate sodium. Three groups of commercially available dispersion agents are high-molecular-weight (Efka® 4000 Series), low-molecular-weight (Efka® 5000 and Efka® 6000 Series) and polyacrylate polymer dispersants (Dispex®, Pigmentdisperser and Ultradispers® range).

Solubilizing agents act as surfactants and increase the solubility of one agent in another. A substance that would not normally dissolve in a solution can dissolve with the use of a solubilizing agent. One example is Polysorbate 80 (C64H124026, also known as polyoxyethylene-sorbitan-20 mono-oleate, or Tween 80). Another example of a solubilizing agent is Kolliphor® SLS. Kolliphor® SLS can be used as a solubilizer to enhance the solubility of poorly soluble APIs in both solid and liquid oral dosage forms. Kolliphor® SLS grades are also suitable for semi solid dosage forms like creams, lotions and gels. Kolliphor® SLS can be used in physical mixing, melt granulation, spray drying and hot melt extrusion processes.

Sweetening and flavoring agents are compounds that sweeten or add or mask flavour of a pharmaceutical formulation. Examples of sweetening or flavouring agents include but are not limited to glucose, sucrose, saccharin, methyl salicylate, peppermint, and the like. Additional sweetening and flavouring agents are provided below.

Surfactants are amphipathic compounds having lyophobic and lyophilic groups. They can be used to solubilize hydrophobic API in an aqueous solution, or as components in an emulsion, or to aid self-assembly vehicles for oral delivery, or as plasticizers in semi-solid formulations, or to improve API absorption and/or penetration. Examples of surfactants include but are not limited to non-ionic surfactants such as ethers of fatty alcohols. Cationic surfactants may possess antibacterial properties. These include phospholipid lecithin, bile salts, certain fatty acids and their derivatives. Gemini surfactants are effective potential transfection agents for non-viral gene therapy. Ionic liquids may also act as secondary surfactants. Other surfactants include anionic surfactants such as docusate sodium (which may also function as a dispersion agent), and sodium lauryl sulfate (SLS) or other detergent which functions to break surface tension and separate molecules.

Coatings are compounds applied typically to tablets and capsules to provide an outer layer (coat) that can perform one or more functions such as but not limited to enhancing stability (e.g., by preventing or reducing moisture-based deterioration), improving swallowability (e.g., by improving taste and texture), providing or altering color, and altering release profile of the solid form (e.g., by rendering the solid form an immediate release delayed release or extended release form). An example of a coating is an enteric coating which controls where in the digestive tract the API will be released.

Film coated tablets. This disclosure provides tablets that are covered with a layer (optionally a thin layer) or film of a polymeric substance which protects the API from atmospheric conditions and/or masks taste and/or odor of API or other excipients, particularly when such taste and/or odor may be objectionable.

Enteric coatings. Some APIs may be destroyed by gastric juice or may cause irritation to the stomach. These factors can be overcome by coating an oral formulation such as a tablet with a polymeric coating that is insoluble in the stomach environment but readily soluble in the intestinal environment. This results in delay in the disintegration of the oral form until it reaches the small intestine. Like coated tablets, enteric coated tablets should be administered in whole form. Broken or crushed forms of the enteric coated tablets cause destruction of the API by gastric juice or irritation to the stomach.

In some instances, enteric coat (or coating) materials are polymers which contain acidic functional groups capable of being ionized at elevated pH values. At low pH values (e.g. the acidic environment of the stomach), the enteric polymers are not ionized, and therefore insoluble. As the pH increases (e.g., when entering the small intestine), the acidic functional groups ionize and the polymer becomes soluble. Thus, an enteric coating allows a delayed release of the active substance and the absorption of the same through the intestinal mucosa.

Enteric coat materials may comprise an enteric polymer. Enteric coat materials may comprise cellulose, vinyl, and acrylic derivatives. Examples of enteric polymers include but are not limited to cellulose acetate phthalate (CAP), hydroxypropyl methylcellulose phthalate (HPMCP), hydroxypropyl methylcellulose acetate succinate (HPMCAS), polyvinyl acetate phthalate, cellulose acetate trimellitate, polymethacrylic acid, polymethyl methacrylate, and polyethyl methacrylate.

Excipients that may be used in oral liquids, such as oral solutions, suspensions and emulsions, include but are not limited to buffering agents (i.e., buffers), coloring agents, flavoring agents, sweetening agents, preservatives, anti-oxidants, and suspending agents.

Buffering agents are compounds used to control and thus maintain pH of a composition. Examples of suitable buffering agents include carbonate, citrate, phosphate, lactate, gluconate, and tartrate buffering systems.

Coloring agents are compounds that impart or control color of a formulation. Examples of coloring agents may be found in the Handbook of Pharmaceutical Excipients. In some instances, such coloring agents may be soluble in water, and thus may include dyes. If pigments are used, they may need to be dissolved in a non-aqueous solution first and then combined with an aqueous carrier or vehicle if so desired. As example of a coloring agent that is typically used in compounding is amaranth solution at a concentration of about 0.2 to 1% v/v.

Choice of flavoring agent will depend on the taste of the API. In the absence of a flavoring agent, the API may have a salty, bitter, sweet, or sour taste and it may be desirable to include a masking flavor in the formulation. For example, if the taste is salty, then a masking flavor such as apricot, butterscotch, liquorice, peach or vanilla may be used. If the taste is bitter, then a masking flavor such as anise, chocolate, mint, passion fruit or wild cherry may be used. If the taste is sweet, then a masking flavor such as vanilla, fruits or berries may be used. If the taste is sour, then a masking flavor such as citrus fruits, liquorice, raspberry may be used.

Examples of flavoring agents and/or sweetening agents (which in some instances may be one and the same) include syrup (e.g., ˜20% v/v-60% v/v) such as orange syrup (e.g., ˜10-20% v/v) or raspberry syrup (e.g., ˜10-20% v/v), juice including concentrated juice such as concentrated raspberry juice (e.g., ˜2.5-5% v/v), emulsion including concentrated emulsion such as concentrated peppermint emulsion (e.g., ˜2.5% v/v), sugar substitutes such as sorbitol (e.g., 20-35% w/v for oral solutions, 70% w/v for oral suspensions, etc.) or saccharin (e.g., 0.02-0.5% w/v), sodium cyclamate (e.g., 0.01-0.15% w/v), anise water (e.g., 0.5% v/v), concentrated camphor water (e.g., 1% v/v), liquorice liquid extract (e.g., 5% v/v), and glycerol (e.g., up to 20% in alcoholic elixirs).

Preservatives are compounds that increase the long-term stability and thus efficacy of the formulation. One class of preservatives does so by preventing growth of pathogens (e.g., microorganism such as bacteria, mycobacteria and fungi) in the formulation, thereby increasing its shelf life but also improving its safety profile for human or animal use. Liquid formulations having extreme pH values (e.g., less than 3 or greater than 10) or high surfactant concentrations may not need a preservative since they tend to be less conducive for pathogen growth.

Examples of preservatives include ethanol (e.g., ≥10% v/v), benzyl alcohol which tends to have optimal activity at pH less than 5 (e.g., 2.0% v/v), glycerol (or glycerin as the terms are used interchangeably) (e.g., 20% w/v), propylene glycol (e.g., 15-30% w/v), benzoic acid which typically has improved activity at about pH 5, and is slightly soluble in water and freely soluble in ethanol (e.g., 0.01-0.1% w/v in oral solutions or suspensions), sodium benzoate which is freely soluble in water but sparingly soluble in ethanol (e.g., 0.02-0.5% w/v), sorbic acid (e.g., 0.05-0.2% w/v), potassium sorbate (e.g., 0.1-0.2% w/v), parabens (forms of parahydroxybenzoates or esters of parahydroxybenzoic acid), esters of 4-hydroxybenzoic acid (i.e., differing only in the ester group), butylparaben (e.g., 0.006-0.05% w/v for oral solutions and suspensions), ethylparaben (e.g., 0.01-0.05% w/v for oral solutions and suspensions), methylparaben (e.g., 0.015-0.2% w/v for oral solutions and suspensions), propylparaben (e.g., 0.01-0.02% w/v for oral solutions and suspensions).

Anti-oxidants are compounds that prevent oxidation of the formulation or of components of the formulation including most notably the API. Examples of anti-oxidants include ascorbic acid and sodium ascorbate (e.g., 0.1% w/v) and sodium meta-bisulfite (e.g., 0.1% w/v).

Suspending agents are compounds that facilitate and/or improve suspension of one or more components in a liquid. Examples of suspending agents include polysaccharides, water-soluble celluloses, hydrated silicates, and carbopol.

Examples of polysaccharides include acacia gum (e.g., gum arabic, from acacia tree), acacia mucilage, xanthan gum which may be produced by fermentation of glucose or sucrose by the Xanthomonas campestris bacterium, alginic acid which may be prepared from kelp, starch which may be prepared from maize, rice, potato or corn, and tragacanth which may be prepared from Astragalus gummifer or Astragalus tragacanthus.

Acacia gum is often used as a thickening agent for extemporaneously prepared (e.g., compounded) oral suspensions (e.g., at a concentration of 5-15% w/v). It is water soluble, typically at a concentration of about 1 part to about 3 parts water. It may be used in combination with other thickeners as in Compound Tragacanth Powder BP which contains acacia, tragacanth, starch and sucrose.

Alginic acid tends to swell but not dissolve in water due to its ability to absorb 200-300 times its own weight of water, and it thereby imparts a viscous colloidal property to a formulation. Sodium alginate is the most widely used salt and it is often used at a concentration of about 1-5% w/v). Because of its anionic nature, it is typically incompatible with cationic materials.

Starch is slightly soluble to soluble in water. It is typically used in combination with other compounds (e.g., sodium carboxymethylcellulose). As another example, it is one of the constituents of Compound Tragacanth Powder.

Tragacanth is practically insoluble in water but swells rapidly in 10 times its own weight in hot or cold water to produce a viscous colloidal solution or semi-gel. It may takes several days to hydrate fully and achieve maximum viscosity after dispersion in water. It is also regarded as a thixotropic, intending that becomes more fluid upon agitation (e.g., stirring or shaking) and less fluid (and thus more solid-like or semi-solid-like) at rest or upon standing. It is typically dissolved in alcohol such as ethanol first and then combined with water. Compound Tragacanth Powder BP, which includes tragacath along with acacia, starch, and sucrose, may be used in concentrations of about 2-4% w/v.

Water-soluble celluloses include methylcellulose, hydroxyethylcellulose, sodium carboxymethylcellulose, and microcrystalline cellulose.

Methylcellulose is a semisynthetic polysaccharide having the general formula of C6H7O2(OH2)OCH3]n, and it may beproduced by methylation of cellulose. Several grades are available, varying in degree of methylation and chain length. For example, a 2% solution of methylcellulose 20 has a kinematic viscosity of 20 cS, while a 2% solution of methylcellulose 4500 has a kinematic viscosity of 4500 cS. The concentration at which it is used depends on viscosity grade which may range from about 0.5% to about 2%. It tends to be more soluble at higher temperatures (e.g., more soluble in warmer water than in colder water), and as a result it disperses in warmer water and upon cooling with stirring a clear or opalescent viscous solution can be produced. Methylcellulose preparations are best prepared by dispersion in about one-third to one-half the total volume of hot water (e.g., 80-100° C.), followed by addition of the remaining water as ice water or ice.

Hydroxyethylcellulose comprises hydroxyethyl groups instead of methyl groups on backbone cellulose chains. It is soluble in both hot and cold water but is otherwise similar to methylcellulose in other properties.

Sodium carboxymethylcellulose forms a clear solution when dispersed in hot or cold water. It is anionic and therefore incompatible with polyvalent cations. It tends to precipitate at low (acidic) pH. It may be used at concentrations up to about 1%.

Microcrystalline cellulose (e.g., commercially available Avicel™) is a purified, partially depolymerized cellulose having thixotropic properties. It is often used with other cellulose derivatives.

One commercially available oral liquid is Ora-Plus® which comprises 97% water, <1% sodium phosphate monobasic, <1% sodium carboxymethylcellulose, <1% microcrystalline cellulose, <1% xanthan gum, and <1% carrageenan. All percentages reflect a v/v percentage. API would be added to this mixture, for example in a stirring vehicle. The mixture may be a high shear mixture. If necessary, the inclusion of the API may be offset by a reduction in the amount of sweetener, in some instances.

Exemplary but non-limiting excipients that may be used in oral liquid formulations such as solutions and suspensions include Aromatic Elixir USP, Compound Benzaldehyde Elixir NF, Peppermint Water NF, Sorbitol Solution USP, Suspension Structured Vehicle USP, Sugar-free Suspension Structured Vehicle USP, Syrup NF, and Xanthan Gum Solution NF.

Exemplary but non-limiting vehicles that may be used in oral liquid formulations such as solutions and suspensions include acacia syrup; aromatic eriodictyon syrup; cherry syrup; citric acid syrup; cocoa syrup; glycyrrhiza elixir; glycyrrhiza syrup; hydriodic acid syrup; isoalcoholic elixir, low; isoalcoholic elixir, high; orange flower water; orange syrup; raspberry syrup; sarsaparilla compound syrup; tolu syrup and wild cherry syrup. In addition, commercial branded vehicles may be utilized are: Coca-Cola Syrup, Ora-Sweet Syrup Vehicle, Ora-Sweet SF Sugar-Free Syrup Vehicle and Syrpalta. Still another vehicle is SyrSpend, including SyrSpend SF (Sugar Free) and SyrSpend SF Alka.

These and other excipients and vehicles are referenced in the United States Pharmacopeia (USP)/National Formulary (NF).

### Altered Release Formulations

Altered- or modified-release tablets may be uncoated or coated. Such tablets contain certain additives or are prepared in certain ways which, separately or together, modify the rate of release of the API, for example, into the gastrointestinal tract, thereby prolonging the effect of API and reducing the frequency of its administration.

Immediate-release tablets and capsules release the API typically in less than 30 minutes. Extended-release tablets and capsules release the API at a sustained and controlled release rate over a period of time, typically within 8 hours, 12 hours, 16 hours, and 24 hours of administration. Delayed-release tablets and capsules release the pharmaceutical dosage after a set time. The delayed-release tablets and capsules are frequently enteric-coated in order to prevent release in the stomach and, thus, release the dosage in the intestinal track. Sustained release, controlled release, and extended release have pretty much the same meaning and are used interchangeably.

Sustained release forms release API under first order kinetics. For example, if the formulation contains 100 mg and it releases at a 10% rate per unit time, then the API content of the formulation is as follows: 100 mg→90 mg→81 mg→72.9 mg . . . , etc., indicating a 10% release of API with each unit of time.

Controlled release forms release API under zero order kinetics. For example, if the formulation contains 100 mg and it releases 10 mg per unit time, then the API content of the formulation is as follows: 100 mg→90 mg→80 mg→70 mg . . . , etc.

### Capsule Formulations/Compositions

Provided herein are a variety of capsule formulations including powder blend-filled capsules and minitablet-containing capsules. The powder-filled capsules can be manufactured using dry blend methodology, hot melt extrusion methodology, hot melt granulation methodology, or spray dry dispersion methodology. Capsules (as well as tablets) having an altered release profile are also contemplated by this disclosure, examples of which include immediate release, delayed release, and extended release capsules. A variety of capsule types are known in the art. Hydroxypropylmethyl cellulose (HPMC) may be used in place of a two-piece capsule. HPMC may also be used as a film coating or a sustained-release tablet material.

### 1. Delayed Release (DR) Capsules

One class of delayed release (DR) capsules comprise one or more minitablets in a capsule. Minitablets are flat or slightly curved tablets with a diameter in the range of 1.0 to 3.0 mm. They are typically filled into a capsule but may also be compressed into larger tablets.

The minitablets may comprise a DR enteric coating or other coating imparting a modified-release profile to the formulation.

As an example, the DR capsules contain API within an enteric-coated minitablet unit. These minitablets, comprising a particular API load per minitablet (e.g., 10 mg or 50 mg) are encapsulated within a size 0 or 00, two-piece capsule. The capsule may be but is not limited to a hydroxypropyl methylcellulose (HPMC) capsule. The API load per capsule represents the target capsule dose strength.

(a) DR Capsule Composition

The components of the minitablet core comprise the API (in the intended dosage strength), a filler/diluent, a disintegrant, an anti-adhesive, and a lubricant. The components of the DR coating comprise a DR polymer, a plasticizer, and one or more anti-tack agents/flow aids. The components of one particular DR capsule are presented in Table 1. In one embodiment, in the minitablet, the binder/diluent is microcrystalline cellulose, the disintegrant is crospovidone, the anti-tack agent/flow aid id colloidal silicon dioxide, and the lubricant is magnesium stearate (non-bovine). In one embodiment, in the DR coating, the DR polymer is Methacrylic acid copolymer, Type C (Eudragit L100-55), the plasticizer is triethyl citrate, the anti-adhesives agents (also considered an anti-tack agent or flow aid) are colloidal silicon dioxide and talc (sterilized). The capsule size is typically chosen based on the dose size and total volume of excipients. In some instances, it may be an HMPC Brown Capsule Size 00. DR polymers and/or excipients of similar type and function can be used in place of those recited above.

Representative but non-limiting relative proportions (weight by total weight) are shown in Table 1.

TABLE 1
Composition of Compound 1 Drug Substance DR Capsules

| | | | |
|---|---|---|---|
| | | DR | |
| | | Capsule¹ | |
| | | capsule | |
| Ingredient | Function | (% w/w) | Range² |

Mini-tablet Core

| | | | |
|---|---|---|---|
| Compound 1 | Active Pharmaceutical | 75% | 70-80% |
| | Ingredient | | |
| Microcrystalline | Binder/Diluent | 4% | 3-5% |
| Cellulose | | | |
| Crospovidone | Disintegrant | 4% | 3-6% |
| Colloidal | Anti-tack | 2% | 1-3% |
| Silicon Dioxide | agent/Flow aid | | |
| Magnesium | Lubricant | 1% | 0.1-2% |
| Stearate - non | | | |
| bovine | | | |

Delayed Release Coating

| | | | |
|---|---|---|---|
| Methacrylic | Delayed Release | 9% | 5-10% |
| acid copolymer, | Polymer | | |
| Type C | | | |
| (Eudragit L100-55) | | | |
| Triethyl citrate | Plasticizer | 2% | 1-2% |
| Colloidal | Anti-tack | 2% | 1-2% |
| silicon dioxide | agent/Flow aid | | |
| Talc, sterilised | Anti-tack agent | 1% | 1-2% |

Encapsulation

| | | |
|---|---|---|
| HMPC Brown | Capsule Shell | 1 capsule |
| Capsule Size 00 | | |

¹May be used for a variety of dosage strengths including for example 5 mg, 10 mg, 20 mg, 50 mg, 100 mg, 200 mg, etc. without limitation.
²Provided the components total to 100%

Table 2 provides the component mass per mini-tablet for one embodiment of the DR capsule.

TABLE 2
Composition of DR Capsule

| | | | | |
|---|---|---|---|---|
| | | mg/mini- | | Ratio of API |
| Ingredient | Function | tablet | Range | to ingredient |

Mini-tablet Core

| | | | | | |
|---|---|---|---|---|---|
| Compound 1 | Active | 7.00 | 5-10 | mg | 1:1 |
| | Pharmaceutical | | | | |
| | Ingredient | | | | |
| Microcrystalline | Binder/Diluent | 0.36 | 0.1-2 | mg | 1:0.051 |
| cellulose | | | | | |
| Crospovidone | Disintegrant | 0.40 | 0.1-2 | mg | 1:0.057 |
| Colloidal silicon | Anti-tack agent/ | 0.16 | 0.01-0.5 | mg | 1:0.023 |
| dioxide | Flow aid | | | | |
| Magnesium | Lubricant | 0.08 | 0.01-0.5 | mg | 1:0.011 |
| stearate, | | | | | |
| non-bovine | | | | | |

Delayed Release Coat

| | | | | | |
|---|---|---|---|---|---|
| Methacrylic acid | Delayed Release | 0.75 | 0.1-2 | mg | 1:0.107 |
| copolymer, Type C | Polymer | | | | |
| (Eudragit L100-55) | | | | | |
| Triethyl citrate | Plasticizer | 0.15 | 0.01-0.5 | mg | 1:0.021 |
| Colloidal silicon | Anti-tack agent/ | 0.15 | 0.01-0.5 | mg | 1:0.021 |
| dioxide | Flow aid | | | | |
| Talc, sterilised | Anti-tack agent/ | 0.15 | 0.01-0.5 | mg | 1: 0.021 |
| | flow aid | | | | |

(b) DR Capsule Manufacturing Process

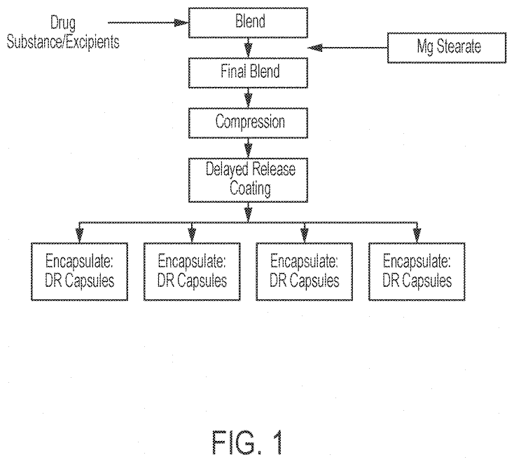

The manufacturing process for the DR capsule involves four distinct processing steps as illustrated in FIG. 1. Briefly, in step one, the mini-tablet components are blended. The anti-adhesive (which may also be referred to herein as an anti-tack agent or a flow aid) (e.g., colloidal silicon dioxide) is mixed with the binder/diluent (e.g., microcrystalline cellulose) and disintegrant (e.g., crospovidone) and then passed through an appropriately sized screen. It is to be understood that in some embodiments provided herein the component selected as the filler may also act as a binder, particularly if the final product is a tablet. The Compound 1 API, is sieved through a 500 micron sieve. Then the API and the excipient mix (e.g., anti-tack agent/flow aid, filler/diluent and disintegrant) are charged to a blender and blended for a defined period of time at a defined rotational speed. Last, the lubricant (e.g., magnesium stearate) is added, and a final blend is completed. In step two, the mini-tablets are tableted. The blend is compressed on a tablet press to a target weight and hardness. In step three, the mini-tablets undergo enteric coating. The mini-tablets are coated on a vented drum coater with the delayed release polymer to achieve a target 15% mini-tablet weight gain. The coated mini-tablets are subsequently heated to remove solvents. In step four, the mini-tablets are encapsulated. The DR coated mini-tablets are encapsulated into the size 1, 0 or 00 two-piece, hydroxypropyl methylcellulose (HPMC) capsule at a weight corresponding to the target active strengths (e.g., 1-1000 mg including but not limited to 10 mg, 50 mg, and 100 mg) DR capsules.

The capsules may be manufactured in their entirety and then shipped to a clinical site or pharmacy. Alternatively, the minitablets may be manufactured and shipped to a clinical site or pharmacy, with or without the capsules, and then the pharmacist may assemble the minitablets into the capsules based on dosage needed for any particular patient. The same process applies for any of the minitablet-containing capsules provided herein.

### 2. Delayed Release/Extended Release (DR/ER) Capsules

The DR/ER capsules contain the API within in one or more minitablet units which have been coated with extended release (ER) and delayed release (DR) polymer layers. These DR/ER mini-tablets, at a defined API load per minitablet, are encapsulated into a size 0, 1 or 00, two-piece capsule such as a hydroxypropyl methylcellulose (HPMC) capsule at the clinical site prior to dosing.

Delayed-release minitablets (and thus capsules) delay release of the API until the minitablet (or capsule) has passed through the stomach to prevent the API from being destroyed or inactivated by gastric juices or where it may irritate the gastric mucosa. Extended-release minitablets (or capsules) function to release and thus make the API available in vivo over an extended period following ingestion.

(a) DR/ER Capsule Composition

The ER capsules use the same mini-tablet cores as used in the DR capsule (see above). Typically, they comprise the API, a diluent (e.g., microcrystalline cellulose), a disintegrant (e.g., crospovidone), an anti-tack agent/flow aid (e.g., colloidal silicon dioxide) and a lubricant (e.g., magnesium stearate).

The mini-tablets are coated initially with an ER polymer and subsequently coated with the same enteric coat used in the DR capsule (see above). The pH independent ER coat consists of a rate controlling polymer (e.g., ammonio methacrylate copolymer, or EUDRAGIT® L100, or EUDRAGIT® S 100, or other methacrylic acid—methyl methacrylate copolymers), a plasticizer (e.g., triethyl citrate), and anti-tack agent/flow aid (e.g., colloidal silicon dioxide and talc), all dispersed in an isopropyl alcohol (IPA)/water solvent mix. The polymer provides the extended-release characteristics of the coating. IPA and water are evaporated during the coating process. The level of the ER polymer coat applied to the mini-tablet cores is targeted between 1% and 11% weight gain of the mini-tablet mass, such that differing in vitro release rates of the active component are achieved.

The ER coated mini-tablets are then coated with a delayed release polymer (e.g., methacrylic acid copolymer, Type C (EUDRAGIT® L100-55)), a plasticizer (e.g., triethyl citrate), and anti-tack agents/flow aids (e.g., colloidal silicon dioxide and talc) at a target weight gain of 15% of the mini-tablet mass.

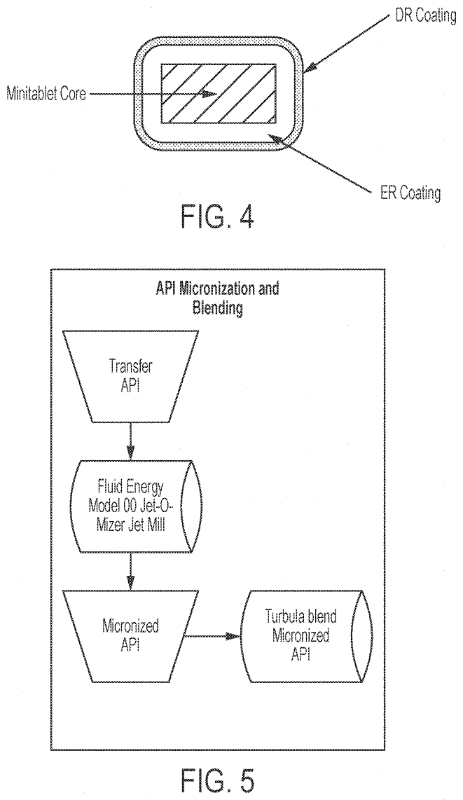

A schematic of the ER mini-tablet is illustrated in FIG. 4. These mini-tablets are encapsulated into a capsule (e.g., an HPMC capsule) at target weights to provide the active dosage form. Exemplary composition of ER capsules is given in Table 4. The composition for Compound 1 ER mini-tablets are given in Table 5. Table 5 provides specific examples of formulation components and amounts however it is to be understood that such amounts may be varied, for example to correspond to the ranges shown in Table 4.

TABLE 4
Composition of Compound 1 ER Capsules.

| | Capsule (% w/w) | | |
|---|---|---|---|
| | ER Slow | ER Medium | ER Fast |
| | (% w/w specific | (% w/w specific | (% w/w specific |
| Ingredient | example and range) | example and range) | example and range) |

Mini-tablet Core

| | | | | |
|---|---|---|---|---|
| Compound 1 | Active | (68.55%) | (71.78%) | (74.60%) |
| | Pharmaceutical | 65-70% | 70-73% | 73-80% |
| | Ingredient | | | |
| Microcrystalline | Binder/Diluent | (3.53%) | (3.69%) | (3.84%) |
| Cellulose | | 3-4% | 3-4% | 3-4% |
| Crospovidone | Disintegrant | (3.92%) | (4.10%) | (4.26%) |
| | | 3.5-4.5% | 3.5-4.5% | 3.5-4.5% |
| Colloidal | Anti-tack agent/ | (1.57%) | (1.64%) | (1.71%) |
| silicon dioxide | Flow aid | 1-2% | 1-2% | 1-2% |
| Magnesium | Lubricant | (0.78%) | (0.82%) | (0.85%) |
| Stearate, | | 0.25-1% | 0.5.1% | 0.5-1% |
| non-bovine | | | | |

Extended Release Coating

| | | | | |
|---|---|---|---|---|
| Triethyl citrate | Plasticizer | (0.52%) | (0.295%) | (0.11%) |
| | | 0.1-0.75% | 0.1-0.5% | 0.05-0.25% |
| Colloidal | Anti-tack agent/ | (1.46%) | (0.84%) | (0.29%) |
| silicon dioxide | Flow aid | 1-2% | 0.5-1% | 0.1-0.5% |
| Talc, sterilised | Anti-tack agent | (1.46%) | (0.84%) | (0.29%) |
| | | 1-2% | 0.5-1% | 0.1-0.5% |
| Ammonio | Rate controlling | (5.17%) | (2.95%) | (1.02%) |
| Methacrylate | polymer | 4.5-5.5% | 2.5-3.5% | 0.75-1.25% |
| Copolymer, Type A | | | | |
| (Eudragit RLPO) | | | | |

Delayed Release Coating

| | | | | |
|---|---|---|---|---|
| Methacrylic acid | Delayed Release | (8.15%) | (8.15%) | (8.15%) |
| copolymer, Type C | polymer | 7.5-8.5% | 7.5-8.5% | 7.5-8.5% |
| (Eudragit L100-55) | | | | |
| Triethyl citrate | Plasticizer | (1.63%) | (1.63%) | (1.63%) |
| | | 1-2% | 1-2% | 1-2% |
| Colloidal | Anti-tack agent/ | (1.63%) | (1.63%) | (1.63%) |
| silicon dioxide | Flow aid | 1-2% | 1-2% | 1-2% |
| Talc, sterilised | Anti-tack agent | (1.63%) | (1.63%) | (1.63%) |
| | | 1-2% | 1-2% | 1-2% |
| HMPC Brown | Capsule Shell | 1 capsule | 1 capsule | 1 capsule |
| Capsule Size 00 | | | | |

TABLE 5
Composition for Compound 1 ER Mini-tablets.

| | | | | |
|---|---|---|---|---|
| | | ER Slow | ER Medium | ER Fast |
| | | mg/mini-tablet | (mg/mini-tablet) | mg/mini-tablet) |
| | | (ratio of API | (ratio of API | (ratio of API to |
| Ingredient | Function | to component) | to component) | component) |

Mini-tablet Core

| | | | | |
|---|---|---|---|---|
| Compound 1 | Active | 7.00 | 7.00 | 7.00 |
| | Pharmaceutical | (1:1) | (1:1) | (1:1) |
| | Ingredient | | | |
| Microcrystalline | Binder/Diluent | 0.36 | 0.36 | 0.36 |
| Cellulose | | (1:0.051) | (1:0.051) | (1:0.051) |
| Crospovidone | Disintegrant | 0.40 | 0.40 | 0.40 |
| | | (1:0.057) | (1:0.057) | (1:0.057) |
| Colloidal | Anti-tack agent/ | 0.16 | 0.16 | 0.16 |
| silicon dioxide | Flow aid | (1:0.023) | (1:0.023) | (1:0.023) |
| Magnesium | Lubricant | 0.08 | 0.08 | 0.08 |
| Stearate, non- | | (1:0.011) | (1:0.011) | (1:0.011) |
| bovine | | | | |

Extended Release Coating

| | | | | |
|---|---|---|---|---|
| Triethyl citrate | Plasticizer | 0.053 | 0.029 | 0.01 |
| | | (1:0.0076) | (1:0.004) | (1:0.0014) |
| Colloidal | Anti-tack agent/ | 0.15 | 0.082 | 0.027 |
| silicon dioxide | Flow aid | (1:0.021) | (1:0.012) | (1:0.0039) |
| Talc, sterilised | Anti-tack agent | 0.15 | 0.082 | 0.027 |
| | | (1:0.021) | (1:0.012) | (1:0.0039) |
| Ammonio | Rate | 0.528 | 0.288 | 0.096 |
| Methacrylate | controlling | (1:0.075) | (1:0.041) | (1:0.014) |
| Copolymer, | polymer | | | |
| Type A | | | | |
| (Eudragit RLPO) | | | | |

Delayed Release Coating

| | | | | |
|---|---|---|---|---|
| Methacrylic | Delayed | 0.833 | 0.795 | 0.765 |
| acid copolymer, | Release | (1:0.119) | (1:0.114) | (1:0.109) |
| Type C | polymer | | | |
| (Eudragit L100-55) | | | | |
| Triethyl citrate | Plasticizer | 0.167 | 0.159 | 0.153 |
| | | (1:0.024) | (1:0.023) | (1:0.022) |
| Colloidal | Anti-tack agent/ | 0.167 | 0.159 | 0.153 |
| silicon dioxide | Flow aid | (1:0.024) | (1:0.023) | (1:0.022) |
| Talc, sterilised | Anti-tack agent | 0.167 | 0.159 | 0.153 |
| | | (1:0.024) | (1:0.023) | (1:0.022) |

It is to be understood with respect to Table 5 and all other similar Tables provided herein that the amount of each excipient may be determined using the exemplary ratio of weight of excipient to weight of API (as provided in the Table), and thus the amount of each excipient may be varied accordingly based on the API weight of the particular formulation.

(b) DR/ER Capsule Manufacturing Process

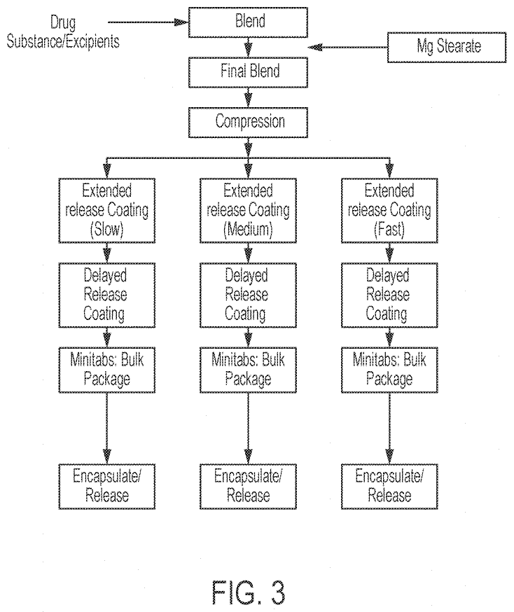

The manufacturing process for DR/ER capsules involves five distinct processing steps as illustrated in FIG. 3. In step one, the mini-tablet components are blended. The anti-tack agent/flow aid (e.g., colloidal silicon dioxide) is mixed with the diluent (e.g., microcrystalline cellulose) and the disintegrant (e.g., crospovidone) and then passed through an appropriately sized screen. The API is passed through a 500 micron sieve. Then the API and the excipient mix (e.g., the anti-tack agent/flow aid, the diluent, and the disintegrant) are charged to a blender and blended for a defined period at a defined rotational speed. Finally, the lubricant (e.g., magnesium stearate) is added and a final blend is formed. In step two, the mini-tablets are formed. The blend is compressed on a tablet press to a target weight and hardness. In step three, the mini-tablets are coated with extended release (ER) coating. The mini-tablet cores are coated for example, on a vented drum coater, to target polymer levels ranging from 1% to 10% mini-tablet weight gain. The target polymer levels are achieved by the degree to which the minitablets are sprayed (e.g., the length of time they are sprayed will be proportional to the amount of coating). As will be understood, the greater the coating, the more delayed or extended the release profile of the API. The coated mini-tablets are subsequently heated to remove solvents. In step four, the ER mini-tablets undergo DR enteric coating. The ER coated mini-tablets are further coated, for example on a vented drum coater, with the DR polymer to achieve a target 15% mini-tablet weight gain. Then the coated mini-tablets are subsequently heated to remove solvents. In step five, the minitablets are encapsulated.

### 3. Dry Blend Capsules

(a) Dry Blend Capsule Composition

In one embodiment, the dry blend capsule comprises the Hsp90 inhibitor, a filler/diluent, a disintegrant, a lubricant, and a capsule. The filler/diluent may be microcrystalline cellulose, NF (such as Avicel PH112). The disintegrant may be croscarmellose sodium, NF (such as Ac-Di-Sol). The lubricant may be magnesium stearate, NF, Ph.Eur. (vegetable source—Grade 905-G). Similar methodology may be used to make tablets provided a sufficient amount of binder is used, and the resultant powder is tableted.

Table 3 provides the quantitative composition for an exemplary 100 mg strength dry blend capsule.

TABLE 3
Composition of a Compound 1 100 mg strength capsule.

| | | Amount per | | | |
|---|---|---|---|---|---|
| | | Capsule (100 | | | |
| Component | Function | mg strength) | | Range | |
| Compound 1 | API | 100 | mg | 10-100 | mg |
| Microcrystalline | Diluent | 297 | mg | 250-350 | mg |
| Cellulose, NF (Avicel | | | | | |
| PH112) | | | | | |
| Croscarmellose | Water- | 2 | mg | 1-5 | mg |
| Sodium, NF (Ac-Di- | absorbing | | | | |
| Sol) | agent; capsule | | | | |
| | disintegrant | | | | |
| Magnesium Stearate, | Lubricant | 1 | mg | 0.1-2 | mg |
| NF, Ph.Eur. | | | | | |
| (Vegetable Source - | | | | | |
| Grade 905-G) | | | | | |

| | | |
|---|---|---|
| Total | 400 | mg |

| | | |
|---|---|---|
| Size 0, hard-gelatin | 1 capsule | 1 capsule |
| white opaque capsule | | |

(b) Dry Blend Capsule Manufacturing Process

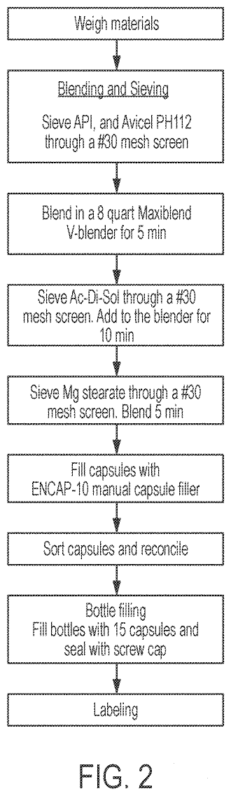

FIG. 2 illustrates an exemplary manufacturing process for a dry blend capsule.

The manufacturing process for a Compound 1 capsule is outlined below. First the components are weighed. Next, the components are blended and sieved. Specifically, the API and the diluent are sieved through a #30 mesh screen, and then blended (e.g., in an 8 quart Maxiblend V-blender) for 5 minutes. The disintegrant is then sieved through a #30 mesh screen, and added to the blender, and the mixture is blended for another 10 minutes. Next the lubricant is sieved through a #30 mesh screen, and added to the blender, and the mixture is blended for another 5 minutes. The capsules are then filled (e.g., with an ENCAP-10 manual capsule filler) with the blended mixture before being sorted and reconciled. The bottles are filled with a defined number (e.g., 15) capsules and sealed with a screw cap before labeling.

### 4. Hot Melt Extrusion (HME) Capsules

(a) HME Capsule Composition

Polymers that may be used in the manufacture of HME capsules are given in Table 6. In this methodology, a combination of API and a predetermined amount of one such polymer are used to form an extrudate. The extrudate is then blended with remaining excipients to product capsules. Examples of such excipients are also provided in Table 6. It will be understood that a similar methodology can be used to make tablets provided the formulation comprises a sufficient amount of binder (for tableting purposes). Such tablets may be coated or uncoated.

TABLE 6
Polymers Used in the Manufacture of HME Capsules.

| Polymer | Brand |
|---|---|
| Vinylpyrollidone:vinylacetate Copolymer | Kollidon ® VA 64 |
| Vinylpyrrolidone | Kollidon ® K 30 |
| Methacrylic Acid Copolymer, Type C | Eudragit ® L100-55 |
| Amino Methacrylate Copolymer | Eudragit ® E PO |
| Hypromellose Acetate Succinate | HPMCAS-MF |
| Hypromellose | HPMC E5 |

Excipients Used with Extrudates in Formulation of Capsules

| | |
|---|---|
| Docusate Sodium | — |
| (anionic surfactant that can act as emulsifying, | |
| wetting and/or dispersion | |
| Sodium Lauryl Sulfate | SLS |
| (detergent and surfactant, breaks surface tension | |
| and separates molecules) | |
| Croscarmellose Sodium | Ac-Di-Sol |
| (internally cross-linked sodium | |
| carboxymethylcellulose for use as a | |
| superdisintegrant) | |
| Gelatin Capsules, Size 1, White Opaque | Coni-Snap |

Exemplary compositions of the HME Capsules are given in Table 7. The 10.0 mg dose strength represents a sample dose.

TABLE 7
Exemplary Composition of HME Capsules.

| | | | Ratio of |
|---|---|---|---|
| | 10.0 mg dose | | API to |
| Material¹ | strength | | ingredient |
| Compound 1 (drug substance)¹ | 10 | mg | 1:1 |
| Povidone (KOLLIDON ® K30)¹ | 30 | mg | 1:2-5 |
| (HME polymer) | | | |
| Microcrystalline cellulose (AVICEL ® | 70 | mg | 1:3-7 |
| PH-101) (diluent) | | | |
| Croscarmellose sodium | 10 | mg | 1:0.5-1.5 |
| (disintegrant) | | | |
| Magnesium stearate | 1 | mg | 1:0.01-1.0 |
| (lubricant) | | | |
| White Opaque Size 0 or 00 gelatin | 1 capsule | | |
| capsules | | | |
| Total: | 121 | mg | |

¹Added as a 1:3 ratio API/HME polymer extrudate powder (40 mg/capsule).

(b) HME Capsule Manufacturing Process

The HME capsules are manufactured using the following procedure. In step one, the API and disintegrant (e.g., KOLLIDON® K30) are dispensed and screened (e.g., using a 18 mesh screen). Disintegrants may be used to disperse solid forms and make the API available for adsorption, by for example avoiding clumping in the stomach, etc. In step two, the mixture undergoes high sheer mixing. The mixture is then further mixed, for example in a GMX Mixer. In step three, the API/disintegrant blend from step two undergoes melt extrusion for example with a Leistritz 18-mm extruder. The extrudate is pelletized in-line. In step four, the pelletized extrudate is milled for example with a Fitzmill L1A and a 0.02 inch screen at 10,000 rpm and screened through a 60 mesh screen to give a milled material. In step five, a diluent (e.g., microcrystalline cellulose) and another disintegrant (e.g., croscarmellose sodium) are added to the milled material from step four. The mixture is screened using a 18 mesh sieve. In step six, primary dilution blending of the mixture from step five in a bin blender of suitable size is performed for 10-60 minutes at 10-50 rpm. In step seven, a lubricant (e.g., magnesium stearate) is added to the mixture from step six and the resultant mixture is then passed through a 30-mesh screen. In step 8, encapsulation is performed using for example an InCap with Powder Dosing Unit to the specified target weight. In step 9, an inspection and release test is performed. The capsules are inspected by pre-determined test methods.

### 5. Hot Melt Granulation (HMG) Capsules

(a) HMG capsule composition

An HMG capsule may comprise API, a binder/solubilizing agent (e.g., Gelucire 50/13), a diluent (e.g., Lactose 316 (Fast Flo) Monohydrate), and a disintegrant (e.g., Ac-Di-Sol® SD-711, croscarmellose sodium). A similar strategy could be used to make tablets provided a sufficient amount of binder is used and the resultant granulation is tableted.

Exemplary compositions of HMG capsules of different dosage strengths are provided in Table 8.

TABLE 8
Composition of Compound 1 Capsule.

| | | Quantity per | | Quantity per | | Quantity per | |
|---|---|---|---|---|---|---|---|
| | | Capsule | | Capsule | | Capsule | |
| Ingredient | Function | (10 mg) | | (50 mg) | | (200 mg) | |
| NDI-010976 | Active Ingredient | 10.00 | mg | 50.00 | mg | 200.00 | mg |
| drug substance | (API) | | | | | | |
| Gelucire 50/13 | Binder/Solubilizing | 90.00 | mg | 90.00 | mg | 90.00 | mg |
| | Agent | | | | | | |
| Lactose 316 | Diluent | 327.50 | mg | 287.50 | mg | 137.50 | mg |
| (Fast Flo) | | | | | | | |
| Monohydrate | | | | | | | |
| Ac-Di-Sol ® | Disintegrant | 22.50 | mg | 22.50 | mg | 22.50 | mg |
| SD- 711 | | | | | | | |
| Croscarmellose | | | | | | | |
| Sodium | | | | | | | |

| | | | | | | |
|---|---|---|---|---|---|---|
| Total Mass | 450.00 | mg | 450.00 | mg | 450.00 | mg |

Each formulation may then be encapsulated in for example a size 0 white opaque coni-snap capsule.

(b) HMG Capsule Manufacturing Process

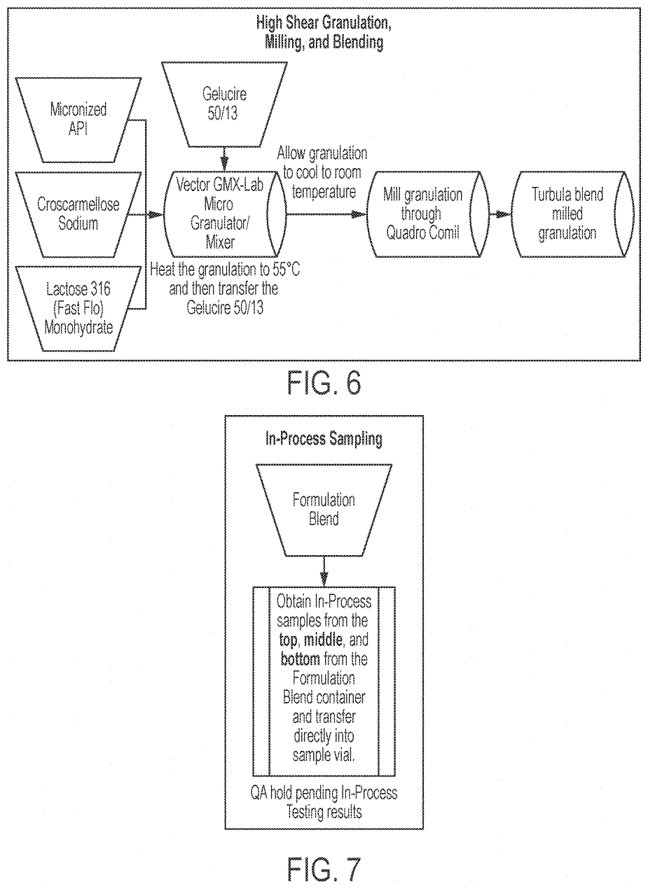

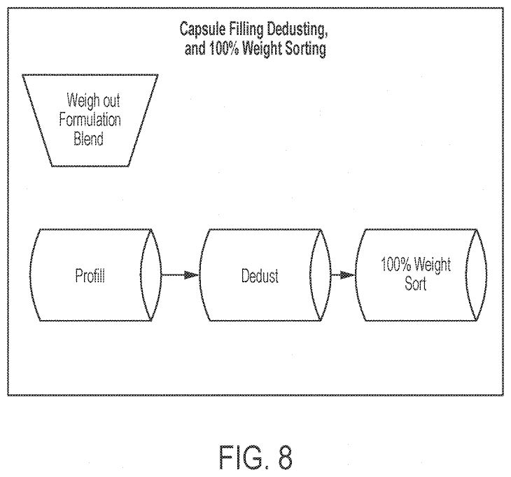

The manufacturing process for HMG capsules involves the following steps. First, the API undergoes micronization. This process is illustrated in FIG. 5. Next, the micronized API undergoes hot melt high shear granulation, milling, and blending. This is illustrated in FIG. 6. Then, the API undergoes in-process sampling as shown in FIG. 7. Finally, the API undergoes capsule filling, dedusting, and 100% weight sorting. This is illustrated in FIG. 8. FIGS. 5-8 and the narratives below describe the manufacturing process for multiple dosage strengths filled into capsules.

It is to be understood that a similar manufacturing process may be used to generate tablets. In this instance, the final powder would be compacted and formed into tablets. In some instances, it may be beneficial to add a binder for example to the final HME powder, then blend and compact into tablets. The binder helps to achieve cohesiveness of the powder in the tableted form.

Micronization. API particle size is reduced for example using a Fluid Energy Jet-O-Mizer, Model 00, 2 inch vertical loop jet mill. The compressed air supply may be high purity nitrogen with a sufficient inlet pressure (e.g., at least 100-200 psi). The pusher nozzle and grinder nozzle pressures are both maintained at 50-100 psi throughout the milling process. The feed rate may be controlled by a vibratory feeder, at an equipment set point of 4. Approximately 1000 grams of material is generated over the course of approximately 6 hours by continuously feeding. This material is then collected in a single container and mixed prior to incorporation into the hot melt granulations at for example 10 mg, 50 mg, and 100 mg dosage strengths.

Hot Melt High Shear Granulation, Milling, and Blending. The granulations are prepared for example in a jacketed 4-L bowl on a Vector GMX Lab-Micro High Shear granulator. The bowl is jacketed with water at 60° C. Approximately half of the filler (e.g., lactose monohydrate), disintegrant (e.g., croscarmellose sodium), and the micronized API are added to the bowl. The remaining filler (e.g., lactose monohydrate) is then used to dry wash the API transfer container prior to addition to the bowl. The dry, solid components are then mixed until the blend reaches 55° C. Once this temperature is reached, a binder/solubilizing agent (e.g., Gelucire 50/13) is added and the chopper is engaged. An immediate temperature drop occurs as the binder/solubilizing agent (e.g., Gelucire 50/13) melts, and the granulation continues mixing until the product temperature recovers to 55° C. to ensure complete melting and mixing of for example Gelucire 50/13. This granulated product is then allowed to cool to room temperature. The cooled granulation is milled for example using a Quadro Comil 197S equipped with a 1905 m screen and a round impeller.

Gelucire 50/13 is a non-ionic, water dispersible surfactant comprised of PEG-esters, a small glyceride fraction and free PEG. It is able to self-emulsify on contact with aqueous media thereby forming a fine dispersion (e.g., a microemulsion (SMEDDS)). It can also act as a solubilizer/wetting agent in which case it improves the solubility and wettability of APIs in vitro and in vivo. It can further act as a bioavailability enhancer leading to improved in vivo drug solubilization that ultimately facilitates absorption. It has also been shown to have good thermoplasticity and thus can be used as a binder in melt processes.

Capsule Filling, Dedusting, and 100% Weight Sorting. The powder is encapsulated, for example using a Profill apparatus, into size 0 white opaque gelatin capsules and dedusted. The final capsule drug product has a fill weight of 450 mg, of which 90 mg is Gelucire 50/13, 22.5 mg is Croscarmellose Sodium, and the remaining weight is comprised of Lactose Monohydrate and micronized API. The amount of Lactose and Compound 1 drug substance are dependent on the dosage strength, and are adjusted as needed to achieve a desired fill weight for each strength.

### 6. Hot Granulation and Dry Blend Capsule Compositions

Capsule formations may be manufactured using micronization and hot melt granulation. Additional capsule formulations are contemplated including for example the following:

(1) API (i.e., Hsp90 inhibitor) and Ac-Di-Sol Capsules,

(2) API and Na Starch Glycolate Capsules

(3) Hot Melt Micronized API and Glycerol Monostearate Capsules

(4) Hot Melt Micronized API and Gelucire Capsules

(5) Hot Melt Micronized API and Vitamin E TPGS Capsules

(6) Hot Melt API and Glycerol Monostearate Capsules

(7) Hot Melt API and Gelucire Capsules

(8) Hot Melt API and Vitamin E TPGS Capsules

(9) Micronized API only

(10) Micronized API Blend Capsules

(11) Hot Melt Micronized API and Gelucire Capsules.

In another embodiment, the capsule formulation comprises the API, a filler (e.g., MCC), and a disintegrant (e.g., Ac-Di-Sol), optionally in a weight ratio of 40% to 40% to 20%. Other ranges of excipients are provided in Table 8-1.

TABLE 8-1
Compound 1 API and Ac-Di-Sol Capsule Formulation

| Component | % Composition | Range³ |
|---|---|---|
| Compound 1 API | 40 | 20-60% |
| MCC (filler) | 40 | 30-60% |
| Ac-Di-Sol (disintegrant) | 20 | 10-40% |
| Total | 100 | 100% |

³Provided the contents total 100%

In a related embodiment, the API may be micronized. Thus, the capsule formulation may comprise the micronized API, a filler (e.g., MCC), a disintegrant (e.g., Ac-Di-Sol), optionally in a weight ratio of 25.5% to 64.5% to 10%. Other ranges of excipients are provided in Table 8-2.

TABLE 8-2
Micronized API Blend Capsule Formulation

| Component | % Composition | Range⁴ |
|---|---|---|
| Micronized Compound 1 API | 25.5 | 10-50% |
| MCC (filler) | 64.5 | 40-80% |
| Ac-Di-Sol (disintegrant) | 10 | 5-30% |
| Total | 100 | 100% |

⁴Provided the contents total 100%

In another embodiment, the capsule formation comprises the API, a filler (e.g., MCC), and a disintegrant (e.g., sodium starch glycolate), optionally in a weight ratio of 40% to 40% to 20%. Other ranges of excipients are provided in Table 8-3.

TABLE 8-3
Compound 1 API and Na Starch Glycolate Capsule Formulation

| Component | % Composition | Range⁵ |
|---|---|---|
| Compound 1 API | 40 | 10-50% |
| MCC (filler) | 40 | 40-80% |
| Na Starch Glycolate | 20 | 5-30% |
| Total | 100 | 100% |

⁵Provided the contents total 100%

Other capsule formulations may comprise hot melt micronized API. An example of such a capsule formulation comprises hot melt micronized API, a filler (e.g., MCC), a disintegrant (e.g., Ac-Di-Sol), and an emulsifier (e.g., glycerol monostearate), optionally in a weight ratio of 25.5% to 44.5% to 10% to 20%. Other ranges of excipients are provided in Table 8-4.

TABLE 8-4
Hot Melt Micronized API and Glycerol
Monostearate Capsule Formulation

| Component | % Composition | Range⁶ |
|---|---|---|
| Micronized Compound 1 API | 25.5 | 10-50% |
| MCC (filler) | 44.5 | 40-80% |
| Ac-Di-Sol (disintegrant) | 10 | 1-10% |
| Glycerol Mono stearate | 20 | 10-20% |
| Total | 100 | 100% |

⁶Provided the contents total 100%.

Another example of such a capsule formulation comprises hot melt micronized API, a filler (e.g., MCC), a disintegrant (e.g., Ac-Di-Sol), and a binder/solubilizing agent (e.g., Gelucire 50/13, a non-ionic, water dispersible surfactant composed of well-characterized PEG-esters, a small glyceride fraction and free PEG), optionally in a weight ratio of 25.5% to 44.5% to 10% to 20%. Other ranges of excipients are provided in Table 8-5.

TABLE 8-5
Hot Melt Micronized API and Gelucire Capsule Formulation

| Component | % Composition | Range⁷ |
|---|---|---|
| Micronized Compound 1 API | 25.5 | 10-50% |
| MCC (filler) | 44.5 | 40-80% |
| Ac-Di-Sol (disintegrant) | 10 | 1-10% |
| Gelucire 50/13 | 20 | 10-20% |
| Total | 100 | 100% |

⁷Provided the contents total 100%

Another example of such a capsule formulation comprises hot melt micronized API, a filler (e.g., MCC), a disintegrant (e.g., Ac-Di-Sol), and vitamin E TPGS, optionally in a weight ratio of 25.5% to 44.5% to 10% to 20%. Other ranges of excipients are provided in Table 8-6.

TABLE 8-6
Hot Melt Micronized API and Vitamin
E TPGS Capsule Formulation

| | % | Weight per Unit | |
|---|---|---|---|
| Component | Composition | (mg) | Range⁸ |
| Micronized Compound 1 | 25.5 | 102 | 10-50% |
| (API) | | | |
| MCC (filler) | 44.5 | 178 | 40-80% |
| Ac-Di-Sol (disintegrant) | 10 | 40 | 1-10% |
| Vitamin E TPGS | 20 | 80 | 10-20% |
| Total | 100 | 400 | 100% |

⁸Provided the contents total 100%.

Other capsule formulations may comprise a hot melt API. An example of such a capsule formulation comprised hot melt API, a filler (e.g., MCC), a disintegrant (e.g., Ac-Di-Sol), and an emulsifier (e.g., glycerol monostearate), optionally in a weight ratio of 25.5% to 44.5% to 10% to 20%. Other ranges of excipients are provided in Table 8-7.

TABLE 8-7
Hot Melt Compound 1 API and Glycerol
Monostearate Capsule Formulation

| Component | % Composition | Range⁹ |
|---|---|---|
| Compound 1 API | 25.5 | 10-50% |
| MCC (filler) | 44.5 | 40-80% |
| Ac-Di-Sol (disintegrant) | 10 | 1-10% |
| Glycerol Mono stearate | 20 | 10-20% |
| Total | 100 | 100% |

⁹Provided the contents total 100%.

Another example of such a capsule formulation comprises hot melt API, a filler (e.g., MCC), a disintegrant (e.g., Ac-Di-Sol), and a binder/solubilizing agent (e.g., Gelucire 50/13), optionally in a weight ratio of 25.5% to 44.5% to 10% to 20%. Other ranges of excipients are provided in Table 8-8.

TABLE 8-8
Hot Melt Compound 1 API and Gelucire Capsule Formulation

| Component | % Composition | Range¹⁰ |
|---|---|---|
| Compound 1 API | 25.5 | 10-50% |
| MCC (filler) | 44.5 | 40-80% |
| Ac-Di-Sol (disintegrant) | 10 | 1-10% |
| Gelucire 50/13 | 20 | 10-20% |
| Total | 100 | 100% |

¹⁰Provided the contents total 100%.

Another example of such a capsule formulation comprises hot melt API, a filler (e.g., MCC), a disintegrant (e.g., Ac-Di-Sol), and vitamin E TPGS, optionally in a weight ratio of 25.5% to 44.5% to 10% to 20%. Other ranges of excipients are provided in Table 8-9.

TABLE 8-9
Hot Melt Compound 1 API and Vitamin
E TPGS Capsule Formulation

| Component | % Composition | Range¹¹ |
|---|---|---|
| Compound 1 API | 25.5 | 10-50% |
| MCC (filler) | 44.5 | 40-80% |
| Ac-Di-Sol (disintegrant) | 10 | 1-10% |
| Vitamin E TPGS | 20 | 10-20% |
| Total | 100 | 100% |

¹¹Provided the contents total 100%.

### 7. Spray Dry Dispersion (SDD) Capsules and Tablets

(a) SDD Capsule and Tablet Composition

SDD tablets may be prepared by spray drying a water-soluble polymer with an API. The SDD is then blended with excipients to control dissolution, disintegration, and release of the active ingredient.

Dispersion can be manufactured using a variety of water-soluble polymers including for example HPMCAS (HPMCAS (AFFINISOL™): Hypromellose Acetate Succinate), PVP VA (PVP VA (Kollidon Va. 64): Polyvinylpyrrolidone/vinyl acetate) and PVP K30 (PVP K30 (average MW 40,000): Polyvinylpyrrolidone). Table 9 provides examples of various API dispersions using these polymers and at different ratios.

TABLE 9
Compound 1 Dispersions

| | SDD | | | | | | |
|---|---|---|---|---|---|---|---|
| | HPMC AS:Compound 1 | | | PVP VA:Compound 1 | | | PVP K30:Compound 1 |
| Drug Load | 1:1 | 2:1 | 3:1 | 1:1 | 2:1 | 3:1 | 1:1 (capsule SDD) |

Compositions of API SDD prototype tablets using PVP VA as an exemplary water-soluble polymer (Dispersions+Excipients) are shown in Table 10. The batch formulae for API SDD are given in Table 11. The batch formulae for 100 mg API tablets is given in Table 12.

TABLE 10
Composition of Compound 1 SDD Prototype Tablets Using PVP VA (Dispersions + Excipients).

| | | Prototype Tablets | | | | | | | | | | | |
|---|---|---|---|---|---|---|---|---|---|---|---|---|---|
| Components (mg) | | 1 | 2 | 3 | 4 | 5 | 6 | 7 | 8 | 9 | 10 | 11 | 12 |
| Intra- | 3:1 PVP VA:Compound 1 | 400 | 400 | 400 | 400 | 400 | 400 | 400 | 400 | 400 | 400 | 400 | 400 |
| Granular | Sodium Bicarbonate | 120 | 160 | 80 | 0 | 0 | 0 | 120 | 80 | 100 | 120 | 120 | 120 |
| | (buffering agent) | | | | | | | | | | | | |
| | Kollidon CL | 0 | 0 | 0 | 30 | 40 | 20 | 30 | 30 | 37.5 | 30 | 30 | 30 |
| | (superdisintegrant and | | | | | | | | | | | | |
| | dissolution enhancer) | | | | | | | | | | | | |
| | NaCl | 0 | 0 | 0 | 0 | 0 | 0 | 0 | 0 | 0 | 40 | 0 | 40 |
| | (carrier, dissolution agent) | | | | | | | | | | | | |
| | microcrystalline cellulose | 66 | 66 | 66 | 194 | 184 | 204 | 36 | 36 | 45 | 16 | 16 | 0 |
| | (filler) | | | | | | | | | | | | |
| | SLS | 16 | 16 | 16 | 16 | 16 | 16 | 16 | 16 | 20 | 16 | 16 | 16 |
| | (detergent and surfactant) | | | | | | | | | | | | |
| | Sub-Total: | 602 | 642 | 562 | 640 | 640 | 640 | 602 | 562 | 602.5 | 622 | 582 | 606 |
| Extra- | Microcrystalline cellulose | 66 | 66 | 66 | 118 | 128 | 108 | 36 | 36 | 45 | 16 | 16 | 0 |
| Granular | (filler) | | | | | | | | | | | | |
| | Sodium Bicarbonate | 120 | 80 | 160 | 0 | 0 | 0 | 120 | 160 | 200 | 120 | 120 | 120 |
| | (buffering agent) | | | | | | | | | | | | |
| | NaCl | 0 | 0 | 0 | 0 | 0 | 0 | 0 | 0 | 0 | 0 | 40 | 40 |
| | (carrier, dissolution agent) | | | | | | | | | | | | |
| | Kollidon CL | 0 | 0 | 0 | 30 | 20 | 40 | 30 | 30 | 37.5 | 30 | 30 | 30 |
| | (superdisintegrant and | | | | | | | | | | | | |
| | dissolution enhancer) | | | | | | | | | | | | |
| | Fumed Silica | 8 | 8 | 8 | 8 | 8 | 8 | 8 | 8 | 10 | 8 | 8 | 8 |
| | (thickening agent, anti- | | | | | | | | | | | | |
| | caking agent, free-flow agent) | | | | | | | | | | | | |
| | Mg Stearate | 4 | 4 | 4 | 4 | 4 | 4 | 4 | 4 | 5 | 4 | 4 | 4 |
| | (anti-adherent agent, lubricant) | | | | | | | | | | | | |
| | Sub-Total: | 198 | 158 | 238 | 160 | 160 | 160 | 198 | 238 | 297.5 | 178 | 218 | 202 |

| | | | | | | | | | | | | |
|---|---|---|---|---|---|---|---|---|---|---|---|---|
| Total (mg): | 800 | 800 | 800 | 800 | 800 | 800 | 800 | 800 | 900 | 800 | 800 | 808 |

TABLE 11
Batch Formulae for API SDD.

| Material | SDI Percentage¹² |
|---|---|
| Compound 1 API | 25% |
| Kollidon ® VA 64 Fine | 75% |
| (water-soluble polymer) | |

¹²The SDI percentage ratios may be 1:1, or 1:2 or 1:4 instead of the 1:3 shown in the Table.

TABLE 12
Batch Formulae for 100 mg Tablets using SDI

| | | |
|---|---|---|
| Ingredient | % | Range |

Intra-granular Components

| | | |
|---|---|---|
| Compound 1 SDI | 66 | 40-70 |
| Sodium Hydrogen Carbonate | 20 | 10-25 |
| (Emprove) | | |
| (buffering agent) | | |
| Kollidon CL (Crospovidone) | 5 | 1-5 |
| (superdisintegrant and | | |
| dissolution enhancer) | | |
| Sodium chloride | 7 | 1-10 |
| (carrier, dissolution agent) | | |
| Kolliphor SLS Fine | 2 | 1-3 |
| (solubilizer) | | |
| Intra-granular subtotal (g) | 100 | |

Extra-granular Components

| | | |
|---|---|---|
| Sodium Hydrogen Carbonate | 60 | 40-70 |
| (buffering agent) | | |
| Sodium chloride | 15 | 5-20 |
| (carrier, dissolution agent) | | |
| Kollidon CL (Crospovidone) | 20 | 5-30 |
| (superdisintegrant and | | |
| dissolution enhancer) | | |
| Fumed silica (e.g., Aerosil | 4 | 1-5 |
| 200) | | |
| (thickening agent, anti-caking | | |
| agent, free-flow agent) | | |
| Sodium Stearyl Fumarate | 1 | .1-2 |
| (e.g., PRUV (JRS)) | | |
| (lubricant) | | |
| Extra-granular subtotal (g) | 100 | |

Tablet Coating Components

| | |
|---|---|
| Opadry II white (other colors | 1-20% weight |
| may be used) | gain |
| Sterile Water for Injection¹³ | |

¹³SWI is removed during manufacture and thus not part of the final formulation.

Opadry II is an excipient that is dissolved in water. The resultant solution is then sprayed on the tablets. The tablets are then dried and then considered “coated”. It is primarily used for tablet protection, i.e. stability from moisture as an example, but providing immediate release just as could be achieved from an uncoated tablet. Other colors may be used for identification purposes.

(b) SDD Capsule and Tablet Manufacturing Process

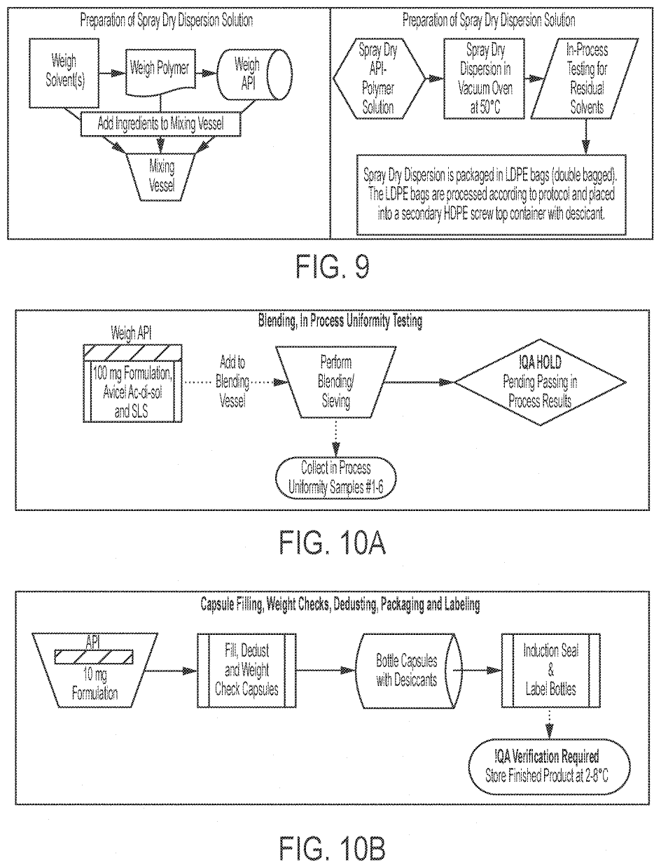

The manufacturing process for both API capsules and tablets requires the generation of a spray dried dispersion (SDD). FIG. 9 describes the general manufacturing process to produce Compound 1 dispersions.

The following procedure is manufacturing a 100 mg dose strength API capsule using spray dry dispersion. An organic solvent (e.g., methylene chloride, acetone, methanol, ethanol, and the like) is gravimetrically dispensed into a 20-L mixing vessel. While mixing with a top down mixer generating a medium vortex, the requisite mass of API and water-soluble polymer (e.g., Povidone (Kollidon 30)), for example at ratios of 1:1, 1:2, 1:3, or 1:4, are rapidly added to a defined volume of the organic solvent (e.g., methylene chloride). The API/water-soluble polymer mixture is readily soluble in the organic solvent (e.g., methylene chloride), and is mixed for a minimum of one hour to ensure complete dissolution.

Using a peristaltic pump, the solution is pumped for example through the Buchi B290 two fluid spray nozzle into the drier at approximately 0.5-5 kg/hour using for example compressed nitrogen as the atomizing gas. The spray drier's inlet drying gas temperature is adjust to maintain on outlet temperature of approximately 40-50° C., depending on the solvent used, throughout the spray drying process. Finally, all the spray dried powder is collected and transferred to drying trays and placed in a vacuum oven for until all solvent is removed.

Tablet SDD. Solvents are gravimetrically dispensed into a mixing vessel. While mixing with a top down mixer generating a medium vortex, a defined mass of the water soluble polymer (e.g., PVP VA 64 polymer) is slowly added to the defined volume of mixing solvent (e.g., a 1:1 methylene chloride: methanol mixture) and stirred for a defined period of time. The solution is observed to ensure all solids are dissolved. A defined mass of API is added while mixing. The solution is mixed for a minimum of 2 hours but not more than 4 hours.

The resulting solution is spray dried for example on a GEA Niro Mobile Minor Closed Cycle Spray Dryer using a pressure nozzle and 0.2 mm nozzle tip with a feed rate of approximately 5 kg/hour. Exemplary but non-limiting spray parameters are listed in Table 13. All the spray dried powder is collected and transferred to drying trays and placed in a vacuum oven for ˜3 days or at least 60 hours. The materials are held at 50° C. with −25 inches Hg vacuum throughout the drying time.

TABLE 13
Exemplary and Non-Limiting Mobile Minor Spray Parameters
Mobile Minor Spray Parameters

| | |
|---|---|
| Inlet Temperature | Automatic Mode, 150° C. |
| Condenser | Automatic Mode, −8° C. |
| Preheater | Automatic Mode, 35° C. |
| Feed Pump | Active: 3.3 mm |
| | Wash: 2.2 mm |
| Nozzle Pressure | 500-700 PSI |
| Feed Rate | 80-90 g/min |
| Outlet Temp | 65-72° C. |

In-Process Control. After drying is complete each tray is sampled for residual solvents testing using a gas chromatography, applying the USP limit specifications for the solvents used. In addition, each tray is sampled and tested for strength using a UV/V as the potency-indicating method. The strength result is used to set the required dispersion load.

Blend and Encapsulation. The manufacturing process for API blending is shown in FIG. 10A and encapsulation of API capsules is shown in FIG. 10B. Approximately 1650 grams of a 1:1 polymer to API (e.g., PVP:Compound 1) spray dried dispersion is mixed with approximately 1650 grams of microcrystalline cellulose (filler/diluent), 675 grams of croscarmellose sodium (superdistintegrant) and 75 grams of sodium lauryl sulfate (surfactant). The material is blended via Turbula blender.

In-Process Control. The blend may be analyzed for strength (assay) and uniformity. Once in-process specifications are met, the material may be roller compacted on a Vector TFC-220 pilot scale roller compactor. The resulting ribbon may be milled through a 1575 μm screen using a Quadro Comil 197S. The milled powder may be filled into size 00 white gelatin capsules. The target fill weight may be 500 mg for an active dosage strength of 100 mg.

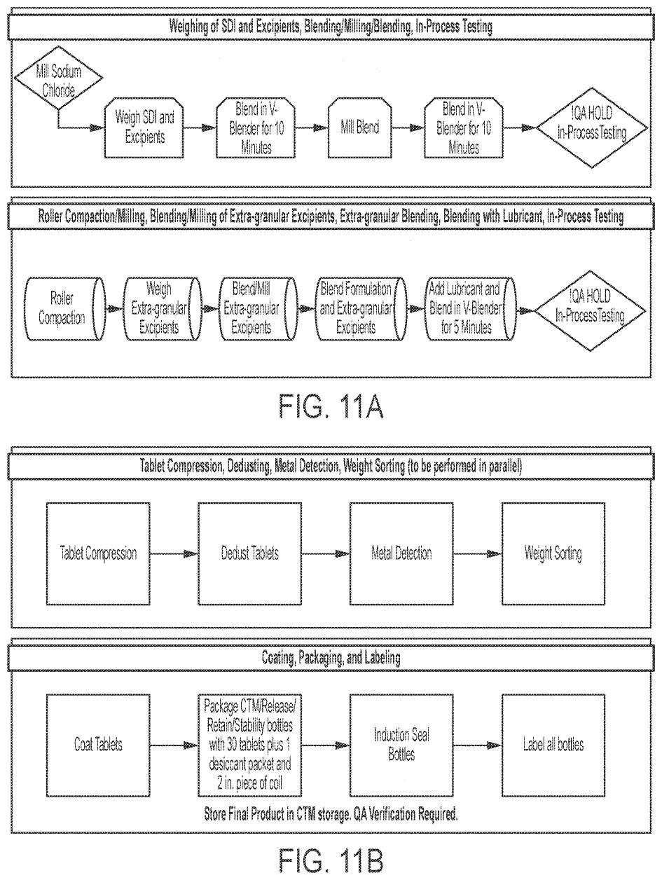

Blend and Tableting. FIGS. 11A and 11B illustrates the manufacturing process for API blend (FIG. 11A) and tableting (FIG. 11B). Sodium chloride (˜1620 g) is milled through a 457 μm round flat screen using a Quadro Comil 187S with round impeller. Sodium chloride may be used as a carrier in solid dispersions to enhance dissolution rates. The intra-granular components are transferred to a 2 cubic foot V-shell in the following order; Compound 1 SDI (2700 g), sodium hydrogen carbonate (810 g), Kollidon CL (405 g), sodium chloride (540 g), sodium lauryl sulfate (216 g) and Compound 1 SDI (2700 g). The SDI transfer container is dried washed with sodium hydrogen carbonate (810 g) and that material is transferred to the V-shell. The intra-granular components are blended for 10 minutes using a GlobePharma MaxiBlend pilot scale blender. The resulting material is milled through a 1143 μm round flat screen using a Quadro Comil 187S with round impeller and subsequently passed through an 850 am stainless steel sieve. The resulting material is again blended for 10 minutes using a GlobePharma MaxiBlend pilot scale blender.

In-Process Control. The blend is analyzed for potency (assay) and uniformity. Once in-process specifications are met, the material is roller compacted on a Gerteis Mini-Pactor. The extra-granular components are transferred to 16 Qt. V-shell in the following order; roller compacted formulation (4032 g), sodium hydrogen carbonate (1597 g), Kollidon CL (399 g), sodium chloride (532 g), Aerosil (1064 g) and roller compacted formulation (4032 g). The intra-granular components are blended for 10 minutes using a Patterson-Kelley V-blender. The resulting material is milled through an 1143 m round flat screen using a Quadro Comil 187S with round impeller, and subsequently passed through an 850 am stainless steel sieve. The resulting material is again blended for 10 minutes using a Patterson-Kelley V-blender.

The API formulation is blended with PRUV (54 g) for 5 minutes using a Patterson-Kelley V-blender with 16 Qt. V-shell for xx minutes. Compound 1 100 mg tablets are manufactured using a Korsch XL100 Tablet Press. Compound 1 formulation blend is loaded into the hopper and settings for fill depth (8.3 mm), edge thickness (2.3 mm) and turret speed (30 rpm) are set up and adjusted on the Korsch XL100. The press is run for two revolutions and start-up tablets are collected for evaluation of physical appearance (100% visual inspection), weight, thickness and hardness. Adjustments to the fill depth, thickness and turret speed are made as needed to approximate the target weight and hardness. Once the start-up is complete and target tablet parameters (weight, thickness and hardness) are met, the Korsch XL100 is started and tableting begins. During tableting, spot-checks for weight, thickness and hardness are performed. A 100% visual inspection of Compound 1 tablets is performed throughout the tableting process and acceptable tablets are dedusted using a CPT TD-400 Deduster, and passed through a Loma/Lock Metal Detector, acceptable tablets are coated with Opadryl II white using Vector LDCS Hi-Coater.

### 8. Wet Granulation—Dry Blend (WG-DB) Tablets

(a) WG-DB Tablet Composition

Tablets made using wet granulation-dry blend (WG-DB) methodology comprise API as well as one or more fillers (or bulking agents) (e.g., lactose, microcrystalline cellulose, mannitol and/or povidone) as intra-granular components. Representative amounts (w/w) of the API and each excipient class are as follows: 20-40% or 20-30% API, 60-80% bulking agents in total, and 0.5-10%, 0.5-2%, 3-6%, 0-30%, 60-73%, and 33-73% of individual bulking agents.

These tablets may further comprise, as extra-granular components, one or more disintegrants (e.g., hydroxypropyl cellulose, croscarmellose sodium such as Ac-Di-Sol, etc.), one or more lubricants (e.g., fumed silica such as Aerosil), and one or more lubricants (e.g., magnesium stearate, sodium stearyl fumarate such as Pruv, etc.). Representative amounts (w/w) of the API and each excipient class are as follows: 0.5-5% or 3-4% disintegrants, 0.5% eluent, and 1.5-2% lubricant.

Exemplary compositions of granulation/dry blend tablet formulations are provided in Table 14. Similar free-flowing powder methodology may be used to generate capsules.

TABLE 14
Typical Compositions of Granulation/Dry
Blend Tablet Formulations.

| | | | |
|---|---|---|---|
| | | Formulation 1 | Formulation 2 |
| | | Prototype: | Prototype: |
| | | Excipient | Excipient |
| Ingredient | Function | Quantity | Quantity |

Intra-granular

| | | | |
|---|---|---|---|
| Drug | Active | 20-40% | 20-30% |
| Substance | Ingredient | | |
| | (API) | | |
| Lactose | Bulking Agent | 33-73% | 0% |
| Avicel | Bulking Agent | 0-30% | 0% |
| (microcrystalline | | | |
| cellulose) | | | |
| Mannitol | Bulking Agent | 0% | 60-73% |
| Povidone | Binding Agent | 0.5-2.0% | 3-6% |

Extra-Granular

| | | | |
|---|---|---|---|
| Hydroxypropyl | Disintegrant | 3-4% | 0% |
| Cellulose | | | |
| Ac-Di-Sol ® | Disintegrant | 0% | 0.5-5% |
| (Croscarmellose | | | |
| Sodium) | | | |
| Aerosil | Eluent | 0.5% | 0.5% |
| (Fumed Silica) | | | |
| Magnesium Stearate | Lubricant | 1.5% | 0% |
| Pruv | Lubricant | 0% | 1.5-2.0% |
| (Sodium Stearyl | | | |
| Fumarate) | | | |

The WG-DB tablets may be immediate release (IR) tablets. Such tablets may be coated with typical standard coatings such as but not limited to Opadry II White. The WG-DB tablets may be DR tablets. Such tablets may be coated with ACRYL-EZE® Aqueous Acrylic Enteric System or with other DR coatings provided herein or known in the art.

Further exemplary formulations (with weight compositions) of WG-DB tablets are provided in Table 15. The Such tablets comprise API with bulking agents such as mannitol (Parteck M100), povidone (Kollidon K30), disintegrants such as croscarmellose sodium (AC-DI-SOL®), eluents such as fumed silica (Aerosil), and lubricants such as sodium stearyl fumarate (Pruv) as excipients. All tablets may be film-coated with for example Opadry 2 White. Delayed release tablets can be further enteric coated with for example ACRYL-EZE® Aqueous Acrylic Enteric System, White. Alternatively, DR tablets may be made by using only an enteric coating without for example in initial standard coat (such as Opadryl 2 White).

TABLE 15
Composition of WG-DB API Tablet.

| | | | |
|---|---|---|---|
| | | Quantity per | Quantity per |
| | | Tablet | Tablet |
| Ingredient | Function | (100 mg, IR) | (100 mg, DR) |

Intra-granular

| | | | | | |
|---|---|---|---|---|---|
| Compound 1 drug | Active | 114 | mg | 114 | mg |
| Substance | Ingredient | | | | |
| | (API) | | | | |
| Parteck ® M100 | Bulking Agent | 482.24 | mg | 480 | mg |
| (Mannitol) | | | | | |
| Kollidon K30 (Povidone) | Binding Agent | 40.80 | mg | 40 | mg |

Extra-Granular

| | | | | | |
|---|---|---|---|---|---|
| Ac-Di-Sol ® | Disintegrant | 3.40 | mg | 3 | mg |
| (Croscarmellose Sodium) | | | | | |
| Aerosil | Eluent | 3.40 | mg | 3 | mg |
| (Fumed Silica) | | | | | |
| Pruv | Lubricant | 13.60 | mg | 14 | mg |
| (Sodium Stearyl | | | | | |
| Fumarate) | | | | | |

Film Coating Ingredients

| | | | | | |
|---|---|---|---|---|---|
| Opadry 2 White | Coating Agent | 14.0 | mg | 14.0 | mg |
| | (for IR Tablets) | | | | |
| ACRYL-EZE ® | Enteric Coating | 0 | mg | 50 | mg |
| Aqueous Acrylic | Agent (for DR | | | | |
| Enteric System, White | Tablets) | | | | |

| | | | |
|---|---|---|---|
| Purified Water | Solvent | N/A | N/A |

IR = Immediate Release, DR = Delayed Release.

(b) WG-DB Tablet Manufacturing Process

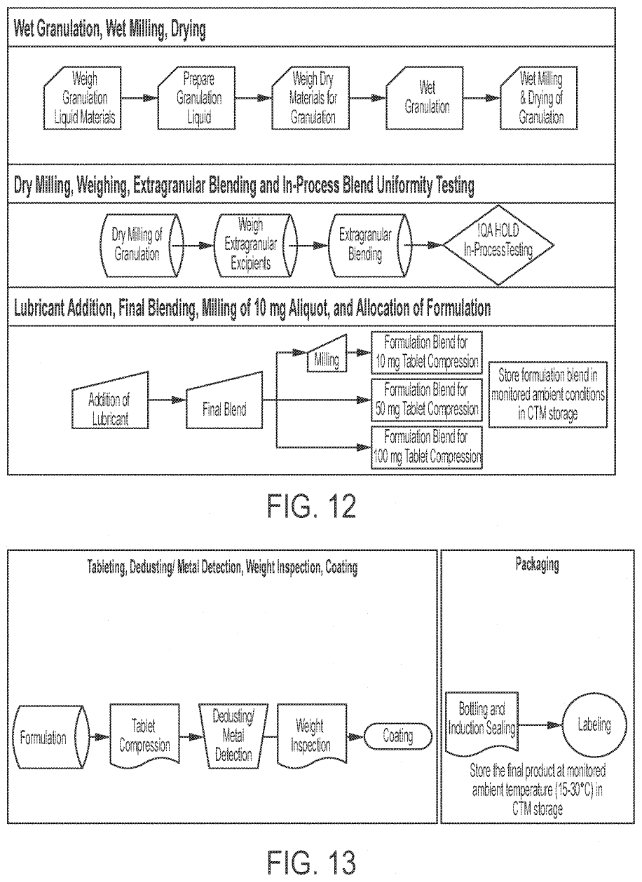

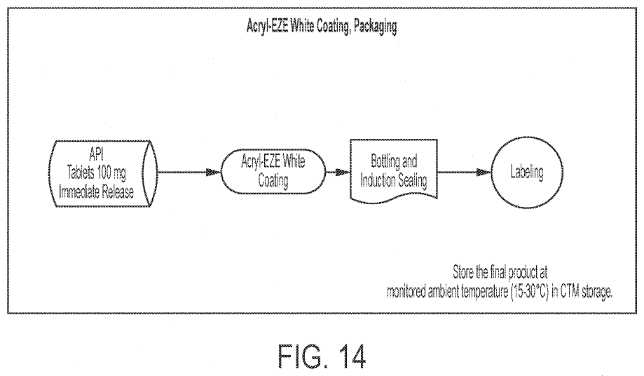

The manufacturing process for WG-DB API tablets involves the manufacture of a wet granulation-common blend for example for the 10 mg, 50 mg, and 100 mg dose strengths, including immediate release tablets. This process is illustrated in FIGS. 12-14. In step one, the excipients are weighed and undergo wet granulation, wet milling, and drying. In step two, the excipients undergo dry milling, weighing, extra-granular blending, and in-process blend uniformity testing. This process is illustrated in FIG. 12. In step three, lubricant is added and the compounds undergo, final blending, milling of a 10 mg aliquot, and allocation of formulation. This is illustrated in FIGS. 12 and 14. In step 4, the compounds undergo tableting, dedusting/metal detection, weigh inspection, coating, and packaging as shown in FIGS. 13 and 14. FIG. 13 shows the tablet compression and coating for 10 mg, 50 mg and 100 mg Compound 1 Immediate Release (IR) tablets.

The following provides an exemplary process for WG-DB immediate release (IR) tablet manufacturing, and is intended to be exemplary and non-limiting in nature.

Weigh Granulation Liquid Materials. Two containers are used to weigh the Kollidon and SWFI. The Kollidon transfer container is placed on to the top loading balance and tared. The required amount of Kollidon is transferred into the Kollidon transfer container and set aside for further processing. The SWFI transfer container is placed on to the top loading balance and tared. The required amount of SWFI is transferred into the SWFI transfer container and set aside for further processing.

Preparation of the Granulation Liquid. The Glas-Col Precision Stirrer is set up with the mixing blade in the container containing the SWFI. The mixing blade is started to create a medium vortex in the SWFI. The container is then labeled as the Granulation Liquid. The Kollidon material is gradually transferred from its container into the Granulation Liquid container. The Kollidon is mixed for at least an hour until the material completely dissolves.

Weigh Dry Materials for Granulation. LDPE bags are used to weigh the Compound 1 drug substance, Mannitol, and Kollidon. Each bag is placed onto the top loading balance and tared, individually. The required amount of Compound 1 drug substance, Mannitol, and Kollidon are transferred into their respective LDPE bags and set aside for further processing.

Wet Granulation. The materials (Compound 1 drug substance, Mannitol and Kollidon) are transferred from the LDPE bags into the bowl for the Vector GMXB-Pilot High Shear Granulator/Mixer. The API, Mannitol, and Kollidon are transferred in the following order: half of the required amount of Mannitol, all of the Kollidon, and all of the Compound 1 drug substance. The LDPE bag that contained the Compound 1 drug substance is then dry washed by transferring the remaining ⅓ of the half of the Kollidon into the empty Compound 1 drug substance LDPE bag. The material is then transferred into the GMXB-Pilot High Shear Granulator/Mixer bowl. The LDPE bag is then dry washed again by transferring the remaining ⅔ of the half of the Kollidon into the empty Compound 1 drug substance LDPE bag and then transferred into the GMXB-Pilot High Shear Granulator/Mixer bowl. The starting gross weight of the Granulation Liquid container is weighed on the balance. The operating settings for the GMXB-Pilot High Shear Granulator/Mixer are entered in the mode display screen. The CCA/Nitrogen source for the operation flow and the pressure are confirmed for the operation of the granulator. The tubing is configured to the inlet on the granulator. The granulation is performed in manual mode. After one minute of dry mixing, the baseline LOD sample is removed and the moisture content of the sample is performed using the Mettler Toledo Moisture Analyzer HB43-S. An LDPE collection bag is then labeled as Granulation. The Granulation bag is then placed on a balance and the tare weight of the bag is obtained. After the tare weight is obtained the Granulation bag is configured to the discharge cylinder of the Vector GMXB-Pilot High Shear Granulator/Mixer and the granulation is discharged. A sample of the granulation from the Granulation bag is removed and the moisture content of the sample is performed using the Mettler Toledo Moisture Analyzer HB43-S. The Granulation bag containing the granulation is then placed on the balance to obtain the gross weight. A calculation is performed to determine the net weight of the granulation by subtracting the previously obtained tare weight of the empty granulation from the gross weight of the Granulation bag. The Granulation Liquid container containing the granulation liquid is then placed on the balance to obtain the gross weight of the granulation liquid container. A calculation is performed to determine the net weight of the granulation by subtracting the previously obtained gross weight of the granulation liquid container.

Wet Milling and Drying of Granulation. The LDPE collection bags are obtained and labeled as Wet Milled granulation. The Quadro Comil 197S is set up with a screen and impeller. The Wet Milled granulation bag is secured to the discharge chute of the Comil. The Comil speed setting is set and the equipment's power switch is turned to the run position. The material from the Granulation bag is rapidly added to the feed chute of the Comil. The material in the Wet Milled Granulation bag is transferred to the warmed fluid bed product bowl. The fluid bed settings are entered and the drying is commenced. When the product bead reaches 40° C., the product bowl is opened and a sample is removed from the fluid bed product bowl for moisture analysis. Based on the moisture analysis result drying continues or drying is stopped. Once the drying has stopped, a LDPE collection bag is labeled as Dry granulation. The Dry Granulation bag is tared on a balance. The product bowl is opened and the material is transferred into the Dry Granulation bag and the weight of the Dry granulation is obtained.

Dry Milling. The LDPE collection bags are obtained and labeled as Dry Milled granulation. The Dry Milled Collection bag is placed on a balance and the tare weight of the empty bag is obtained. The Quadro Comil 197S is set up with a screen and impeller.

The Dry Milled granulation bag is secured to the discharge chute of the Comil. The Comil speed setting is set and the equipment's power switch is turned to the run position. The material from the Dry Granulation bag is rapidly added to the feed chute of the Comil. Any remnant material in the Comil screen is passed through a sieve and transferred to the Dry Milled Granulation bag. The Dry Milled Granulation bag containing the granulation is then placed on the balance to obtain the gross weight. A calculation is performed to determine the net weight of the Dry Milled granulation by subtracting the previously obtained tare weight of the empty Dry Milled granulation bag from the gross weight of the Dry Milled Granulation bag.

Weighing Extra-granular Excipients. Six containers are retrieved to weigh the AC-DI-SOL®, Aerosil, PRUV, Sieved AC-DI-SOL®, Sieved Aerosil, and Sieved PRUV in. The AC-DI-SOL®, Aerosil, and PRUV transfer containers are placed on to the top loading balance and tared, individually. The required amount of the AC-DI-SOL®, Aerosil, PRUV is transferred into their respective transfer containers and set aside for further processing. The Sieved AC-DI-SOL®, Sieved Aerosil, and Sieved PRUV containers are placed on to the top loading balance and tared, individually. The AC-DI-SOL®, Aerosil, and PRUV in the transfer containers are sieved independently and the required amount of sieved material is transferred into the respective Sieved AC-DI-SOL®, Sieved Aerosil, and Sieved PRUV containers and set aside for further processing.

Extra-granular Blending. The GlobePharma Maxi Blend V-Blended is set up with the appropriate V-shell. The material is added to the V-Blender shell in the following order: ½ of the Dry Milled Granulation, all of the sieved AC-DI-SOL®, all of the sieved Aerosil, and the remainder of the half of the dry milled Granulation is added to the V-Blender shell. The GlobePharma Maxi Blend V-Blended is set to blend the material in the V-Blender shell for ten minutes. A Patterson Kelly 1 cubic foot V-Blender was used for a 200 mg blend.

In-Process Testing. Six sample jars are labeled as Compound 1 Final Blend In-process samples (#1-6). The in-process sample jars are placed on a balance and tarred individually. For each sampling jar, a 0.25 mL stainless steel sample thief is used to remove a sample from a specified sample location from the formulation in the V-shell and placed directly into tared sampling jar. The weight of each sample is documented on the sampling jar. The six samples are then submitted for blend uniformity testing. Based on the Blend Uniformity results, the process continues or the GlobePharma Maxi Blend V-Blender is set to blend the material in the V-Blender shell for ten minutes and sampling is repeated with Compound 1 Final Blend.

Additional of Lubrication and Blending. The upper access ports of the GlobePharma Maxi Blend V-Blender are opened and the sieved Pruv is split equally and transferred equally between the two sides of the V-shell. After the addition of the sieved PRUV, the access ports of the GlobePharma Maxi Blend V-Blender are closed and GlobePharma Maxi Blend V-Blender is set to blend the material in the V-Blender shell for three minutes. A Patterson Kelly 1 cubic foot V-Blender was used for a 200 mg blend.

Milling. The required amount of formulation for the 10 mg aliquot is calculated. The LDPE collection bags are obtained and labeled as Milled 10 mg Aliquot. The Milled 10 mg Aliquot is placed on a balance and the tare weight of the empty bag is obtained. The Quadro Comil 197S is set up with a screen and impeller. The Milled 10 mg Aliquot bag is secured to the discharge chute of the Comil. The Comil speed setting is set and the equipment's power switch is turned to the run position. The required amount of formulation for the 10 mg aliquot from the V-Blender is rapidly added to the feed chute of the Comil. Any remnant material in the Comil screen is passed through a sieve and transferred to the Milled 10 mg Aliquot bag. The Milled 10 mg Aliquot bag containing the Milled 10 mg Aliquot is then placed on the balance to obtain the gross weight. A calculation is performed to determine the net weight of the Milled 10 mg Aliquot by subtracting the previously obtained tare weight of the empty Milled 10 mg Aliquot bag from the gross weight of the Milled 10 mg Aliquot.

Formulation Blending for 10 mg, 50 mg and 100 mg Tablets. Six LDPE bags are obtained and placed one inside another to create 3 sets of double LDPE bags. Each inner bags of the three sets are labeled as one of the following: Compound 1 Formulation Blend for Compound 1 Tablets, 10 mg; Compound 1 Formulation Blend for Compound 1 Tablets, 50 mg; and Compound 1 Formulation Blend for Compound 1 Tablets, 100 mg. For each set, the doubled LDPE bags are placed on the balance and tared. The required amount of Formulation Blend to support the 10 mg, 50 mg and 100 mg productions are transferred individually into their respective inner bags. The inner bags containing the formulation blend is secured. Three desiccants are placed into the outer bags, so that the desiccants are positioned between the bags and sealed. The bags are the placed inside of their respective HDPE drum sealed and labeled appropriately.

Tablet Compression. Utilizing the Key International BBTS-10 Rotary Tablet Press the formulation blend is pressed into tablets. The 10 mg tablets are pressed into 5.1 mm round standard concave tablets. The 50 mg tablets are pressed into 9.25 mm round standard concave tablets. The 100 mg tablets are pressed into 9.25 mm×17.78 mm oval tablets. A Korsch XL 100 Tablet Press was used for a 200 mg blend.

Dedusting/Metal Detection. The tablets are passed through the CPT TD-400 Deduster and exit through the exit chute into a tote. The tablets are then passed through the Loma/Lock Metal Detector and collected through the exit chute.

Weight Inspection. The tablets are passed through the SADE SP Weight Sorter and evaluated based on the applicable weight specification.

Coating. The coating solution is prepared with SWFI and Opadry. Utilizing the Vector LDCS HI-Coater, at the applicable spray rate the tablets are coated to achieve the target weight gain. Tablets are evaluated based on the applicable weight specification.

Bottling/Induction Sealing. The coated tablets are packaged eighty count into the applicable size bottle. A desiccant is transferred into the bottle containing the coated tablets. The appropriate size closure is capped onto the applicable bottle. The closure is induction sealed onto the applicable bottle using the Lepel Induction Sealer.

Labeling. The applicable label is visually inspected for absence of smudges. Operators attach one acceptable label to the center location of each bottle. The labeled bottle is inspected to ensure that each bottle contains one label, the label is centered on the bottle, legible and free from damage.

The following provides an exemplary process for WG-DB delayed release (DR) tablet manufacturing, and is intended to be exemplary and non-limiting in nature.

The manufacturing process for DR tablets may involve Acryl-EZE White coating of the IR tablets as manufactured above. The manufacturing process is described in FIG. 14 and involves the following three steps: Acyl-EZE-white coating, bottling and induction sealing, and labeling.

Coating. The coating solution is prepared with SWFI and Acryl-EZE White. Utilizing the Vector LDCS HI-Coater, at the applicable spray rate the tablets are coated to achieve the target weight gain. Tablets are evaluated based on the applicable weight specification.

Bottling/Induction Sealing. The coated tablets are packaged fifty count into the applicable size bottle. A desiccant is transferred into the bottle containing the coated tablets. The appropriate size closure is capped onto the applicable bottle. The closure is induction sealed onto the applicable bottle using the Lepel Induction Sealer.

Labeling. The applicable label is visually inspected for absence of smudges. One acceptable label is attached to the center location of each bottle. The labeled bottle is inspected to ensure that each bottle contains one label and that the label is centered on the bottle, legible, and free from damage.

### 9. Wet Granulation (WG) Capsules.

(a) WG Capsule Composition

Capsules may be manufactured using a wet granulation methodology. When a wetting manufacturing process is used, an excipient is added as a liquid and the powder and liquid are mixed to form for example a paste that is then dried, and can be sieved and blended and/or granulated. The “wet” excipient “complexes” with the API.

As an example, a granulation liquid such as Tween 80 may be used to produce a molecular dispersed form of the API. The granulation formulation may use the following excipients: lubricant such as fumed silica dioxide (e.g., Aerosil V200), filler such as microcrystalline cellulose (e.g., Avicel PH-101), disintegrant and/or binder such as cornstarch, binder and solubilizing agent such as gelatin, Magnesium Stearate, solubilizing agent such as Tween 80, and water. Exemplary quantitative compositions of WG capsules are given in Table 16. The unit formula (50 mg and 100 mg capsules) represent examples of drug substance to excipient load. A similar methodology may be used to generate tablets provided a sufficient amount of binder is used and the granulation is then tableted.

TABLE 16
Quantitative Composition of Compound 1 Capsules

| | Unit Formula | | Unit Formula | | |
|---|---|---|---|---|---|
| Ingredient | (50 mg capsule) | | (100 mg capsule) | | Function |
| Compound 1 drug substance | 50.0 | mg | 100.0 | mg | Active Ingredient |
| White Cornstarch | 40.0 | mg | 80.0 | mg | Inactive Ingredient |
| | | | | | (disintegrant and |
| | | | | | binder) |
| Microcrystalline cellulose | 45.0 | mg | 90.0 | mg | Inactive Ingredient |
| | | | | | (filler) |
| fumed silicon dioxide (Aerosil V200) | 3.0 | mg | 6.0 | mg | Inactive Ingredient |
| | | | | | (lubricant) |
| polysorbate 80 (Tween 80) | 5.0 | mg | 10.0 | mg | Inactive Ingredient |
| | | | | | (solubilizing agent) |
| Gelatin | 2.5 | mg | 5.0 | mg | Inactive Ingredient |
| | | | | | (binder and |
| | | | | | solubilizing agent) |
| Water for injection | as necessary | | as necessary | | Solvent |
| Magnesium stearate | 0.2 | mg | 0.4 | mg | Inactive Ingredient |

| | | | |
|---|---|---|---|
| Capsule | 1 capsule | 1 capsule | Product delivery |

It is to be understood that similar weight ratios can be used to generate capsules comprising more or less API as described herein.

(b) WG Capsule Manufacturing Process

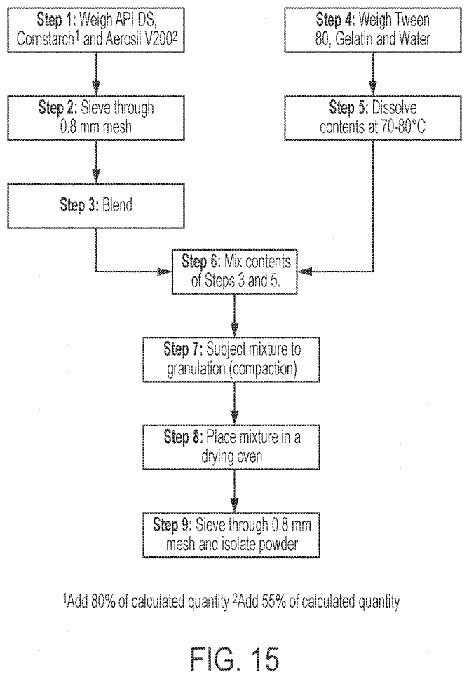

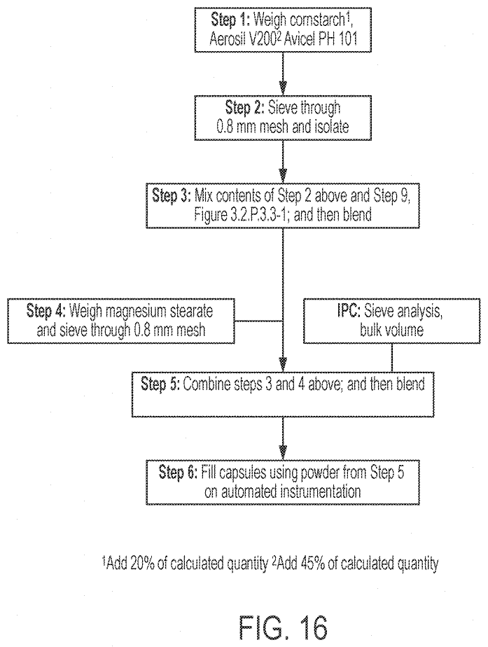

Preparation of Initial Granula. In steps 1-3, the active and inactive compounds are combined. The API, white cornstarch (80% of calculated quantity) and Aerosil V200 (55% of calculated quantity) are passed through a sieve with a mesh size of 0.8 mm, and then combined. The mixture is blended using a Turbula mixer. In steps 4-5, the solution is granulated. Water is added to a separate container and heated between 70-80° C. Tween 80 is added, followed by gelatin. The contents are mixed to form a gelatinous material. In step 6, the mixture undergoes the wetting protocol. The water/Tween 80/gelatin mixture is manually added to the mixture from steps 1-3, which results in a uniform moist mass. In steps 7-9, the mixture undergoes wet granulation. The mixture is granulated and then the mass is dried in an oven (humidity controlled). A free-flowing powder is isolated and passed through a 0.8 mm mesh. A schematic illustrating the preparation of the initial granula is shown in FIG. 15.

Preparation of Capsule Filling Mass/Filling Capsules. In steps 1-2, Cornstarch (20% of calculated quantity), Aerosil V200 (45% of calculated quantity), and Avicel PH-101 are combined and passed through a 0.8 mm mesh and then isolated. In step 3, the mixture is further mixed with the mixture from step 9 above, and then blended. In steps 4-5, magnesium stearate is passed through a 0.8 mm mesh and then added to the contents from step 3 and blended. In in-process control step may also be incorporated here to test the quality of the product. In step 6, the mixture is encapsulated. Hard gelatin capsules, size 2 or size 00, are filled using for example a Zanasi LZ64 capsule filling machine, or an instrument of similar capability. A schematic illustrating the preparation of capsule filling mass/filling capsules is shown in FIG. 16.

### 10. Oral Disintegrating Tablets (ODT)

(a) ODT Compositions

Another example of an oral formulation provided herein is a disintegrating tablet formulation. A disintegrating tablet is an alternative to conventional tablets or capsules. One advantage of disintegrating tablets is improved patient compliance particularly in patients who have difficulty swallowing tablets and capsules generally. Disintegrating tablets are tablets that disintegrate in the oral cavity (mouth).

Such tablets may comprise one or more, including two, three, four, five or more categories of excipients selected from the group consisting of filler/diluent, binder, lubricant, glidant, disintegrating agent, sweetening or flavouring agent, and/or dispersion agent.

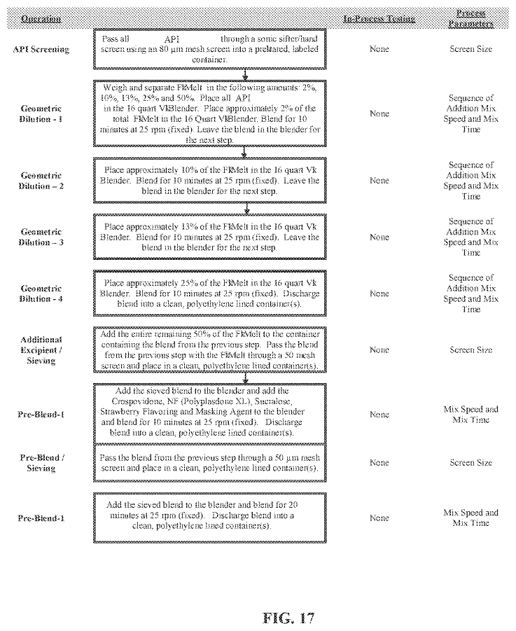

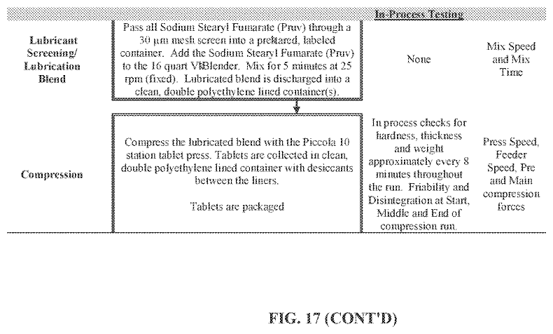

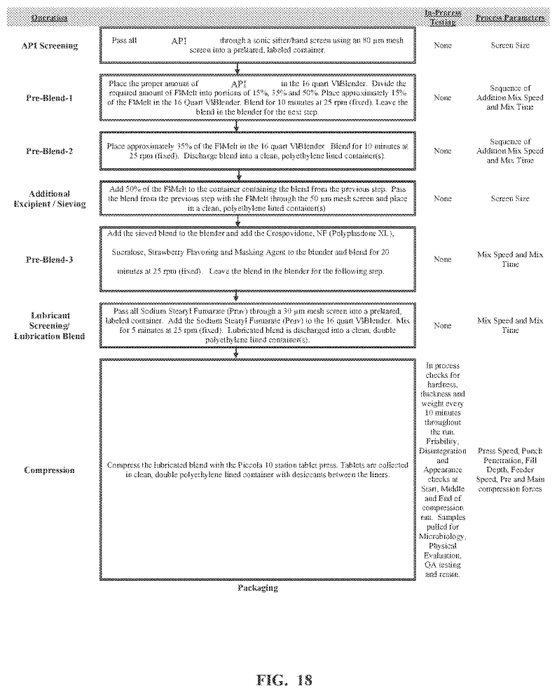

In some exemplary formulations, the oral disintegrating tablets are formulated with 10 mg and 50 mg of API per tablet. There are six excipients in each tablet. An example of the composition of each dosage strength oral disintegrating tablet is provided in Table 17. Schematics for the method of manufacture for oral disintegrating tablets are provided in FIGS. 17 and 18. Tables 18-21 provides examples of ODT excipient combinations and percentages.

TABLE 17
Composition and Quality Standards of Compound
1 Oral Disintegrating Tablets.

| | Amount per Dosage Strength | | | |
|---|---|---|---|---|
| Component | 10 mg | | 50 mg | |
| Compound 1 (drug substance) | 10 | mg | 50 | mg |
| F-Melt | 200 | mg | 200 | mg |
| Crospovidone | 8.0 | mg | 8.0 | mg |
| (disintegrant, also known as | | | | |
| Polyvinylpolypyrrolidone (polyvinyl | | | | |
| polypyrrolidone, PVPP) | | | | |
| Sucralose | 3.0 | mg | 3.0 | mg |
| (sweetener) | | | | |
| Sodium stearyl fumarate | 3.0 | mg | 3.0 | mg |
| (lubricant) | | | | |
| Strawberry flavor | 0.7 | mg | 0.7 | mg |
| Masking flavor | 0.3 | mg | 0.3 | mg |
| (flavoring agent and taste masking | | | | |
| agent) | | | | |
| Target tablet weight (mg) | 225 | mg | 265 | mg |

TABLE 18
Excipient Combinations and Percentages.

| | Filler/Binder | Disintegrant | Lubricant |
|---|---|---|---|
| Formulation | (% Formulation) | (% Formulation) | (% Formulation) |
| 1 | Pearlitol 300DC | Polyplasdone XL | Pruv |
| | (90%) | (8%) | (2%) |
| 2 | Sucrose | Polyplasdone XL | Pruv |
| | (90%) | (8%) | (2%) |
| 3 | Prosolv HD90 | Polyplasdone XL | Pruv |
| | (90%) | (8%) | (2%) |
| 4 | Lactose | Polyplasdone XL | Pruv |
| | (90%) | (8%) | (2%) |

TABLE 19
Excipient Combinations and Percentages Derived
from Formulation 1 from Table 18.

| | Formulation | | | |
|---|---|---|---|---|
| | Filler/Binder | Disintegrant | Lubricant | Glidant |
| Formulation | (% Formulation) | (% Formulation) | (% Formulation) | (% Formulation) |
| 5 | Pearlitol 300DC | Polyplasdone XL | Pruv | Fumed Silica |
| | (90.5%) | (7%) | (2%) | (0.5%) |
| 6 | Pearlitol 300DC | Polyplasdone XL | Pruv | Fumed Silica |
| | (80.5%) | (17%) | (2%) | (0.5%) |
| 7 | Pearlitol 300DC | L-HPC | Pruv | Fumed Silica |
| | (80.5%) | (17%) | (2%) | (0.5%) |

Smaller particle size mannitol (Pearlitol 100SD) can also be used, on the theory that providing a larger surface area allows quicker disintegration. Calcium silicate, a dispersion agent, may be introduced. Exemplary blend excipients are presented in Table 20 below.

TABLE 20
Excipient Combinations and Percentages.

| | Formulation | | | | |
|---|---|---|---|---|---|
| | | | Dispersion | | |
| Formulation | Filler/Binder | Disintegrant | Agent | Lubricant | Glidant |
| number | (%) | (%) | (%) | (%) | (%) |
| 8 | Pearlitol 300DC | Polyplasdone | Calcium | Pruv | Fumed |
| | (57.5%) | XL | Silicate | (2%) | Silica |
| | | (20%) | (20%) | | (0.5%) |
| 9 | Prosolv HD90 | Polyplasdone | Calcium | Pruv | Fumed |
| | (57.7%) | XL | Silicate | (2%) | Silica |
| | | (20%) | (20%) | | (0.5%) |
| 10 | PanExcea | Polyplasdone | n/a | Pruv | Fumed |
| | (82.5%) | XL | | (2%) | Silica |
| | | (15%) | | | (0.5%) |
| 11 | Pearlitol 100SD | Polyplasdone | Calcium | Pruv | Fumed |
| | (57.5%) | XL | Silicate | (2%) | Silica |
| | | (20%) | (20%) | | (0.5%) |
| 12 | Pearlitol 100SD | Polyplasdone | Calcium | Pruv | Fumed |
| | (52.5%) | XL | Silicate | (2%) | Silica |
| | Prosolv HD90 | (15%) | (15%) | | (0.5%) |
| | (15%) | | | | |

(b) ODT Manufacturing Process

Exemplary manufacturing procedures for ODT are as follows:

The excipient components for each blend are weighed and blended in a glass blending vessel at 32 RPM on a Turbula blender for 5 minutes. The powder is then sieved through a 600 m mesh screen and blended for an additional 5 minutes. Each formulation blend is used to produce tablets of a desired dosage strength. Hardness, friability and in vivo disintegration results of these formulations were tested.

All combinations exhibit sufficient hardness, resulting in no friability concerns. Sufficient in-vivo disintegration time is obtained for all formulations. Calcium silicate, used in combination with Prosolv, provide the most rapid disintegration time. However, the mouth feel with Prosolv is poor compared to Pearlitol (mannitol). Tablets prepared with Pearlitol (mannitol) and calcium silicate still provide the quickest disintegration time. Furthermore, they provide the benefit of a cool, smooth mouth feel.

Two additional excipients, F-Melt and Pharmaburst, can also be included. These excipients are compared to a blend consisting of Prosolv, Calcium Silicate, and Polyplasdone XL, as presented in Table 21.

TABLE 21
Excipient Combinations and Percentages

| | Formulation | | | | |
|---|---|---|---|---|---|
| | | | Dispersion | | |
| Formulation | Filler/Binder | Disintegrant | Agent | Lubricant | Glidant |
| number | (%) | (%) | (%) | (%) | (%) |
| 13 | Pharmaburst¹ | n/a | n/a | Lubripharm² | n/a |
| | (98%) | | | (2%) | |
| 14 | F-Melt³ | Polyplasdone | n/a | Pruv | n/a |
| | (93%) | XL | | (2%) | |
| | | (5%) | | | |
| 15 | Mannitol | Polyplasdone | Calcium Silicate | Pruv | Fumed |
| | 300DC | XL | (20%) | (2%) | Silica |
| | (37.5%) | (20%) | | | (0.5%) |
| | Prosolv | | | | |
| | HD90 | | | | |
| | (20%) | | | | |

¹Co-processed mannitol, crospovidone, silica..
²Sodium stearyl fumarate.
³Coprocessed mannitol, crospovidone, anhydrous dicalcium phosphate.

One particular formulation of interest comprises a filler/binder (e.g., F-Melt) at about 90-95% (e.g., 93%), a distintegrant (e.g., Polyplasdone XL) at about 3-7% (e.g., 5%), and a lubricant (e.g., PRUV) at about 1-3% (e.g., 2%).

The excipient components for each blend are weighed and blended in a glass blending vessel at 32 RPM on a Turbula blender for 5 minutes. The powder is then sieved through a 600 μm mesh screen and blended for an additional 5 minutes. Each formulation blend is used to produce 100 mg tablets that were compressed at two different rates. Hardness, friability and in-vivo disintegration properties are then tested for each formulation.

Introduction of Sweeteners and Flavorings and Drug Substance. A sweetener (sucralose) and flavors (orange and/or strawberry) may be added to formulation 14. Following placebo taste testing a combination of sucralose, strawberry flavoring and masking agent were selected. These agents, as well as the API, are combined with the excipients in formulation 14 to produce formulation 16.

The formulation components are weighed and blended in a glass blending vessel at 32 RPM on a Turbula blender for 5 minutes. The powder is then sieved through a 600 μm mesh screen and blended for an additional 5 minutes.

In some embodiments, an orally disintegrating composition such as an orally disintegrating tablet comprises a binder of a filler in an amount of about 75-95% or 75-90% or 75-89% by weight of the total composition, a disintegrating agent in an amount of about 3-4% by weight of the total composition, a sweetener in an amount of about 1 to 1.5% by weight of the total composition, a lubricant in an amount of about 1 to 1.5% by weight of the total composition, and one or more flavouring agents in an amount of about 0.3 to 0.5% by weight of the total composition.

In one specific embodiment, the filler or binder is F-Melt, the disintegrating agent is crospovidone, the sweetening agent is sucralose, the lubricant is sodium stearyl fumarate, and the flavouring agents are strawberry flavour and masking flavour.

In other embodiments, the orally disintegrating composition comprises a filler/binder, a disintegrant, and a lubricant. For example, the filler/binder may be Pearlitol 300DC, sucrose, Prosolv HD90 or lactose, the disintegrant may be polyplasdone XL, and the lubricant may be Pruv. The filler/binder may represent about 75-95% by weight of the total excipients (i.e., inert or non-active components of the formulation). The disintegrant may represent about 5-15% by weight of the total excipients. The lubricant may represent about 0.5-10% by weight of the total excipients. The weight ratio of the filler/binder to disintegrant to lubricant may be 90% to 8% to 2%.

In other embodiments, the orally disintegrating composition comprises a filler/binder, a disintegrant, a lubricant, and a glidant. For example, the filler/binder may be Pearlitol 300DC, the disintegrant may be polyplasdone XL or L-HPC, the lubricant may be Pruv, and the glidant may be fumed silica. The filler/binder may represent about 75-95% by weight of the total excipients (i.e., inert or non-active components of the formulation). The disintegrant may represent about 5-20% by weight of the total excipients. The lubricant may represent about 0.5-10% by weight of the total excipients. The glidant may represent about 0.1 to 5% by weight of the total excipients. The weight ratio of the filler/binder to disintegrant to lubricant to glidant may be 80.5% to 17% to 2% to 0.5% in one instance or 90.5% to 7% to 2% to 0.5% in another instance.

In some embodiments, the composition may comprise PanExcea as a filler/binder, polyplasdone XL or a disintegrant, Pruv as a lubricant, and fumed silica as a glidant. The weight ratio of filler/binder to disintegrant to lubricant to glidant may be 82.5% to 15% to 2% to 0.5%.

In other embodiments, the orally disintegrating composition comprises a filler/binder, a disintegrant, a lubricant, a glidant, and a dispersion agent. For example, the filler/binder may be Pearlitol 300DC or Prosolv HD90 or PanExcea or Pearlitol 100SD or a combination thereof such as Pearlitol 100SD and Prosolv HD90, the disintegrant may be polyplasdone XL, the lubricant may be Pruv, the glidant may be fumed silica, and the dispersion agent may be calcium silicate. The filler/binder may represent about 50-90% by weight of the total excipients (i.e., inert or non-active components of the formulation). The disintegrant may represent about 10-30% by weight of the total excipients. The lubricant may represent about 0.5-5% by weight of the total excipients. The glidant may represent about 0.1 to 2.5% by weight of the total excipients. The dispersion agent may represent about 10-30% by weight of the total excipients. The weight ratio of the filler/binder to disintegrant to lubricant to glidant to dispersion agent may be 57.5% to 20% to 2% to 0.5% to 20%, or 57.7% to 20% to 2% to 0.5% to 20%, or 67.5% to 15% to 2% to 0.5% to 15%.

In other embodiments, the orally disintegrating composition comprises a filler/binder, a disintegrant, a lubricant, a glidant, and a dispersion agent. For example, the filler/binder may be Pharmaburst (co-processed mannitol, crospovidone and silica) or F-Melt (co-processed mannitol, crospovidone, and anhydrous dicalcium phosphate) or a combination of Mannitol 300DC and Prosolv HD90, the disintegrant may be polyplasdone XL, the lubricant may be Lubripharm (sodium stearyl fumarate) or Pruv, the glidant may be fumed silica, and the dispersion agent may be calcium silicate. The filler/binder may represent about 50-99% by weight of the total excipients (i.e., inert or non-active components of the formulation). The disintegrant may represent about 2-25% by weight of the total excipients. The lubricant may represent about 0.5-5% by weight of the total excipients. The glidant may represent about 0.1 to 2.5% by weight of the total excipients. The dispersion agent may represent about 15-25% by weight of the total excipients. The weight ratio of the filler/binder to disintegrant to lubricant to glidant to dispersion agent may be 57.5% to 20% to 2% to 0.5% to 20%.

Other formulations may comprise a filler/binder (e.g., Pharmaburst) and lubricant (e.g., Lubripharm) in a weight ratio of 98% to 2%, wherein these excipients total to 100% the weight of the excipients in the formulation.

Other formulation may comprise a filler/binder (e.g., F-Melt), disintegrant (e.g., polyplasdone XL), and a lubricant in a weight ratio of 93% to 5% to 2%.

Still other formulations may comprise a filler/binder (e.g., a combination of Mannitol 300DC and prosolv HD90 in a weight ratio of 37.5% to 20%), a disintegrant (e.g., polyplasdone XL), a dispersion agent (e.g., calcium silicate), a lubricant (e.g., Pruv), and a glidant (e.g., fumed silica) in a weight ratio of 57.5% to 20% to 20% to 2% to 0.5%.

Any of the foregoing compositions may further include one or more sweetening agents such as but not limited to sucralose and one or more flavoring agents such as but not limited to orange and/or strawberry flavors. Additionally or instead of one or more flavouring agents, a masking agent may be used.

The disintegrating compositions may be made in the following manner: the Hsp90 inhibitor is passed through a sonic sifter or hand screen using an 80 micron mesh screen and into a blender such as a 16 quart V-Blender. The binder/filler (e.g., F-Melt) is added in increments to the active ingredient. Such increments may be for example 2%, 10%, 13%, 25% and 50%. After each addition of filler/binder (up to the 25% addition), the mixture is blended for 10 minutes at 25 rpm, and then the blend remains in the blender throughout the process. Prior to addition of the final 50% of filler/blender, the blend is placed in a clean container (e.g., a polyethylene lined container) and the remaining 50% of the filler/binder is added and the blend is then passed through a 50 micron mesh screen and again placed in a clean container. The sieved blend is then placed in the blender again along with the disintegrant (e.g., polyplasdone XL), sweetening agent (e.g., sucralose), flavouring agent (e.g., strawberry flavouring and masking agent), and this mixture is blended for 10 minutes at 25 rpm. The blend may then be sieved through a 50 micron mesh screen and then again blended for 20 minutes at 25 rpm. The lubricant may be blended separately or together with the final active ingredient containing blend. This may be blended for 5 minutes at 25 rpm. The result is a lubricated blend. This may then be compressed with a tablet press such as a Piccola 10 station tablet press. Tablets so formed may then be stored in clean containers, optionally double polyethylene lined containers, with desiccants between the liners.

The active ingredient dosage strength of these disintegrating tablets may range from about 0.001 to about 1000 mg, including about 0.1 mg to about 500 mg, about 1 mg to about 500 mg, or from about 5 mg to about 100 mg, including for example about 10 mg, about 20 mg, about 30 mg, about 40 mg, about 50 mg, about 60 mg, about 70 mg, about 80 mg, about 90 mg, and about 100 mg dosage strengths. Different dosage strengths are envisioned to address different subject such as for example pediatric versus adult subjects.

### 11. Effervescent Formulations Including Effervescent Tablets

The oral formulation may be an effervescent formulation intending that it may be dissolved in a solution such as an aqueous solution and such solution may then be ingested by the patient.

Effervescent formulations may be manufactured using simple blending of excipients or dry granulation via roller compaction.

Excipients to be used to create the requisite rapidly dissolving table formulation include sodium bicarbonate or calcium bicarbonate, acids such as citric acid, malic acid, tartaric acid, adipic acid, and fumaric acid. Water or other aqueous solution will be used to reconstitute.

### 12. Oral Solutions

Also provided herein are mixed formulations in the form of liquids for oral administration. These may be aqueous solutions, although they are not so limited. They contain one or more active ingredients dissolved in a suitable vehicle.

The solutions may be elixirs or linctuses, for example.

Elixirs are relatively non-viscous, typically clear, flavored orally administered liquids containing one or more active ingredients dissolved in a vehicle that usually contains a high proportion of sucrose or suitable polyhydric alcohol(s) or alcohols. They may also contain ethanol (96 percent) or a dilute ethanol. Polyhydric alcohols are alcohols that contain >1 hydroxyl group. Examples include glycols such as for example propylene glycol (CH3CH(OH)CH2OH); polyethylene glycols (PEGS, macrogols) (OHCH2(CH2CH2O)nCH2OH); and glycerol (CH2OHCHOHCH2OH). Their alcohol content may range from 5-40% (10-80 proof). The concentration of alcohol is determined by the amount required to maintain the API in solution. An example of an elixir is phenobarbital elixir, USP. Elixirs may contain glycerin which acts to enhance their solvent properties and to provide preservative function. Elixirs may be active in the stomach and GI tract.

Linctuses are relatively viscous oral liquids containing one or more active ingredients in solution. The vehicle usually contains a high proportion of sucrose, other sugars or suitable polyhydric alcohol(s). Linctuses may be active in the throat due to their more viscous properties (e.g., as compared to elixirs).

Dissolution of an active ingredient may be improved in a number of ways including for example use of a co-solvent such as ethanol, glycerol, propylene glycol or syrup; modulating or controlling pH throughout the formulation process and/or during storage using for example weak acids or weak bases; solubilization techniques; use of complexation of active ingredients and/or other components; and/or chemical modification of active ingredients and/or other components.

### 13. Oral Suspensions

Oral suspensions are orally administered liquids that contain one or more active ingredients suspended in a suitable vehicle. Certain suspensions are stable for extended periods of time while others may experience separation of the suspended solids from the vehicle, in which case they should be re-dispersed typically by moderate agitation. As with oral solutions, oral suspensions can be particularly advantageous in subjects unable to swallow solid forms such as tablets or capsules. In some instances, it may be preferable to formulate an insoluble derivative of an active ingredient than to formulate its soluble equivalent due to differences in palatability and/or stability.

Availability of active ingredient upon administration of an oral suspension may be improved by reducing suspended particle size, reducing density differences between suspended particle and dispersion medium (carrier or vehicle) (e.g., by addition of sucrose, sorbitol, glucose, glycerol or other soluble, non-toxic components which may be referred to as density modifiers), and/or increasing the viscosity of the dispersion medium (e.g., by addition of a thickening or suspending agent). Certain density modifiers may also be viscosity modifiers. Suspended particle size may change upon storage, particularly if exposed to a temperature fluctuation, with solubility increasing if temperature increases and potential crystallization of the active ingredient if the temperature decreases.

### 14. Compounding Procedures for Oral Formulations

Provided below are exemplary compounding procedures for the preparation of Hsp90 inhibitor oral formulations having a dosage strength in the range of 1-10 mg, including a 2 mg/mL Hsp90 inhibitor liquid formulation and a 2 mg/mL Hsp90 inhibitor suspension in 0.5% methylcellulose. All formulations are prepared using the vehicles listed below:

Vehicle #1-90:10 Labrasol:Vitamin E TPGS (density=1.05 g/mL)

Vehicle #2-90:10 Polyethylene Glycol 400:Vitamin E TPGS (density=1.12 g/mL)

Vehicle #3-0.5% Methylcellulose (400 cps) in Purified Water (density=1.00 g/mL)

The Hsp90 inhibitor (API) may be used as a free form or in a salt form.

Preparation of 2 mg/mL Hsp90 Inhibitor in 90:10 Labrasol: Vitamin E TPGS (Scale: 15 mL):

1. Heat Vehicle #1 (90:10 Labrasol:Vitamin E TPGS) at 60° C. for approximately 10 minutes and mix on a magnetic stir plate. (Vehicle should be a homogenous solution; place back at 60° C. if any visible phase separation of the Vitamin E TPGS is observed.)

2. Weigh 30.0 mg of Hsp90 inhibitor to the compounding container.

3. Weigh 15.75 g of Vehicle #1 to the compounding container.

4. Heat the formulation at 60° C. with occasional vortex mixing to suspend un-dissolved Hsp90 inhibitor. Continue until fully solubilized. (Approximately 5-10 minutes).

Preparation of 2 mg/mL Hsp90 Inhibitor in 90:10 Polyethylene Glycol 400: Vitamin E TPGS (Scale: 15 mL):

1. Heat Vehicle #2 (90:10 Polyethylene Glycol 400:Vitamin E TPGS) at 60° C. for approximately 10 minutes and mix on a magnetic stir plate. (Vehicle should be a homogenous solution; place back at 60° C. if any visible phase separation of the Vitamin E TPGS is seen.)

2. Weigh 30.0 mg of Hsp90 inhibitor to the compounding container.

3. Weigh 16.80 g of Vehicle #2 to the compounding container.

4. Heat the formulation at 60° C. with occasional vortex mixing to suspend un-dissolved Hsp90 inhibitor. Continue until fully solubilized. (Approximately 5-10 minutes).

Preparation of a 2 mg/mL Hsp90 Inhibitor Suspension in 0.5% Methylcellulose (400 Cps) (Scale: 15 mL):

1. Weigh 10.00 g of Vehicle #3 (0.5% methylcellulose) into the compounding container.

2. Weigh 30.0 mg of Hsp90 inhibitor into the compounding container.

3. Weigh an additional 5.00 g of Vehicle #3 to the compounding container on top of the Hsp90 inhibitor.

4. Mix the suspension using a high shear mixer at a speed of 2500 RPM. Move container around the mixing head, up/down and side-to-side, to fully homogenize the suspension. Mix for no less than 20 minutes.

5. Place the suspension on a magnetic stir plate and maintain stirring when removing samples for analysis or dosing.

Alternative preparation procedure for Hsp90 inhibitor in 2 mg/mL in Ora Sweet for clinical compounding:

The following procedure may be used for a variety of dosage strengths including 1-10 mg. Briefly, this procedure involves preparing a small batch of Hsp90 inhibitor in Ora Sweet (or Ora-Blend) using a magnetic stir bar and homogenizer by volumetric dilution. The mixture may be homogenized a 12,000-15,000 for 15 minutes and a 15 g sample may be obtained every 5 minutes for assay. The mixture may be mixed by magnetic stir bar for 15 minutes and a 15 g sample may be obtained every 15 minutes for assay. The mixture may be allowed to stand for 2 hours, then mixed for 10 minutes by magnetic stir bar, following which a 15 g sample may be obtained for assay. More specifically, the following steps may be performed:

Sample Preparation

1. Transfer 1000 mL±2 of Ora sweet to a tared 1 L graduated cylinder.

2. Transfer 250 mL to a 1 L beaker+stir bar and increase the mixing speed until a slight vortex forms.

3. Transfer 2.0 g±0.02 of CF 602 to the beaker and mix for 5 minutes.

4. Insert the homogenizer into the suspension and begin to homogenize a 6,000-8,000 RPM for 5 minutes while mixing.

5. Add 250 mL of Ora Sweet and continue to mix and homogenize for 5 minutes.

6. Add the remaining Ora Sweet

7. Increase the mixing speed to maintain good fluid movement.

8. Increase the homogenizer to 12,000-15,000 for 5 minutes

9. Obtain a 15 g sample from the top and bottom after 5 minutes of homogenization and submit for assay.

10. Discontinue homogenization but continue mixing with the stir bar.

11. Mix for 15 minutes and obtain a 15 g sample to submit for assay.

12. Allow to stand for 2 hours, then mix by magnetic stir bar for 10 minutes. Obtain a 15 g sample from the top and bottom to submit for assay.

13. Re-weigh the graduated cylinder, NMT Tare±10 g (1%)

Then sample and test the various samples using standard assays.

The HME powder described herein may be used in place of the Hsp90 inhibitor alone. Additionally, any USP oral vehicle may be used in place of Ora Sweet including Ora Blend or Ora-Plus or SyrSpend or FlavorSweet.

Suspensions Prepared by HME:

As described herein, HME is a procedure used to generate a powdered form of the API of interest. HME is used when it is desirable to enhance the solubility of the API.

The following describes the preparation of three separate Hsp90 inhibitor formulations:

1) 2 mg/mL Hsp90 inhibitor:PVP K30

2) 2 mg/mL Hsp90 inhibitor:PVP K30 w/ SLS

3) 2 mg/mL Hsp90 inhibitor:PVP K30 w/ Docusate Sodium

Methocel A4M premium is used to prepare the 0.5% methylcellulose (MC) in water vehicle. A mortar and pestle is used to prepare the suspensions.

1) 2 mg/mL Hsp90 inhibitor:PVP K30-30 mL
Pull 30 mL of 0.5% MC vehicle into tared syringe, record weight.
Weigh 273.97 mg of the 25:75 Hsp90 inhibitor:PVP K30 Powder and add to mortar.

Compound suspension with slow addition of MC vehicle to mortar (e.g., add a few drops to form an initial thick paste with pestle, and then add vehicle in small increments to insure uniform mixing and gradual dilution with pestle).

Pull entire suspension formulation up into the original syringe that held vehicle, and transfer from syringe into appropriate container.

$$[ Hsp 90 inhibitor ] , \\ mg \text{/} g = \frac{Wt . of Hsp 90 inhibitor \text{:} PVP K 30 * 0.25 * 0.876}{( Wt . of MC vehicle + Wt . of Hsp 90 inhibitor \text{:} PVP K 30 )}$$

0.25=percent active in formulation

0.876=label claim potency of formulation

2) 2 mg/mL Hsp90 inhibitor:PVP K30 w/SLS—30 mL
Add 6.4 mg of SLS to 35 mL of 0.5% MC vehicle.
Vortex mix to dissolve.
Pull 30 mL of MC/SLS vehicle into tared syringe, record weight.
Weigh 273.97 mg of the 25:75 Hsp90 inhibitor:PVP K30 Powder and add to mortar.

Compound suspension with slow addition of MC/SLS vehicle to mortar (e.g., add a few drops to form an initial thick paste with pestle, and then add vehicle in small increments to insure uniform mixing and gradual dilution with pestle).

Pull entire suspension formulation up into the original syringe that held vehicle, and transfer from syringe into appropriate container.

$$[ Hsp 90 inhibitor ] , \\ mg \text{/} g = \frac{Wt . of Hsp 90 inhibitor \text{:} PVP K 30 * 0.25 * 0.876}{( \begin{matrix}Wt . of MC / SLS vehicle + \\ Wt . of Hsp 90 inhibitor \text{:} PVP K 30\end{matrix} )}$$

3) 2 mg/mL Hsp90 inhibitor:PVP K30 w/ Docusate Sodium—30 mL
Add 6.4 mg of Docusate Sodium (DSS) to 35 mL of 0.5% MC vehicle.
Vortex mix to dissolve.
Pull 30 mL of MC/DSS vehicle into tared syringe, record weight.
Weigh 273.97 mg of the 25:75 Compound 1:PVP K30 Powder and add to mortar.

Compound suspension with slow addition of MC/DSS vehicle to mortar (e.g., add a few drops to form an initial thick paste with pestle, and then add vehicle in small increments to insure uniform mixing and gradual dilution with pestle).

Pull entire suspension formulation up into the original syringe that held vehicle, and transfer from syringe into appropriate container.

$$[ Hsp 90 inhibitor ] , \\ mg \text{/} g = \frac{Wt . of Hsp 90 inhibitor \text{:} PVP K 30 * 0.25 * 0.876}{( \begin{matrix}Wt . of MC / SLS vehicle + \\ Wt . of Hsp 90 inhibitor \text{:} PVP K 30\end{matrix} )}$$

Manufacture of Hsp90 inhibitor oral drinking solution, 100 mg

One exemplary dose of oral drinking solution contains the following:

Active component

| | | |
|---|---|---|
| Hsp90 inhibitor | 100.0 | mg |

Excipients

| Lactic acid | 1 molar equivalent | |
|---|---|---|
| Glucose | 1 | g |
| Passion fruit aroma | 0.150 | g |
| Water | 200 | ml |

Ranges for the above active component and excipients may vary by 0.1 to 100-fold, in some instance, and the excipients may be substituted with like excipients where desired.

Production Method:

Weigh 100 mg Hsp90 inhibitor into container 1. Add 100 ml of water and stir until all contents dissolve or are nearly all dissolved. In a separate container 2 add 100 ml water then add glucose. Stir until all contents dissolve. Add lactic acid and stir until all contents dissolve, followed by passion fruit aroma. Stir for 5-30 min. Add contents of container 1 to container 2. Stir 5-30 min. Dose is ready for administration.

### Subjects and Indications

The subjects to be treated and for whom the oral formulations provided herein are intended include mammals such as humans and animals such as non-human primates, agricultural animals (e.g., cow, pig, sheep, goat, horse, rabbit, etc.), companion animals (e.g., dog, cat, etc.), and rodents (e.g., rat, mouse, etc.). Preferred subjects are human subjects. Subjects may be referred to herein as patients in some instances.

The active compounds and oral formulations provided herein are intended for use in subjects in need of Hsp90 inhibition. Such subjects may have or may be at risk of developing a condition characterized by the presence or the elevated (compared to normal cells) presence of Hsp90 or which may benefit from inhibition of Hsp90 activity. Such conditions may be characterized by the presence of misfolded proteins. Such conditions include without limitation cancer, neurodegenerative disorder, inflammation (or inflammatory conditions) such as but not limited to cardiovascular diseases (e.g., atherosclerosis), autoimmune diseases, and the like.

Cancer

The term “cancer” or “neoplastic disorder” refers to a tumor resulting from abnormal or uncontrolled cellular growth. Examples of cancers include but are not limited to breast cancers (e.g., ER+/HER2− breast cancer, ER+/HER2+ breast cancer, ER−/HER2+ breast cancer, triple negative breast cancer, etc.), colon cancers, colorectal cancers, prostate cancers, ovarian cancers, pancreatic cancers, lung cancers, gastric cancers, esophageal cancers, glioma cancers, and hematologic malignancies. Examples of neoplastic disorders include but are not limited to hematopoietic disorders, such as the myeloproliferative disorders, essential thrombocytosis, thrombocythemia, angiogenic myeloid metaplasia, polycythemia vera, myelofibrosis, myelofibrosis with myeloid metaplasia, chronic idiopathic myelofibrosis, the cytopenias, and pre-malignant myelodysplastic syndromes. In some instances, the indication to be treated is pancreatic cancer, breast cancer, prostate cancer, skin cancer (e.g., melanoma, basal cell carcinoma), B cell lymphoma, Hodgkin's lymphoma, and non-Hodgkin's lymphoma. In some instances, the indication to be treated is pancreatic cancer. In some instances, the indication to be treated is breast cancer. The cancer to be treated may be a primary cancer (without indication of metastasis) or a metastatic stage cancer.

The term “hematologic malignancy” refers to cancer of the bone marrow and lymphatic tissue-body's blood-forming and immune system. Examples of hematological malignancies include but are not limited to myelodysplasia, lymphomas, leukemias, lymphomas (non-Hodgkin's lymphoma), Hodgkin's disease (also known as Hodgkin's lymphoma), and myeloma, such as acute lymphocytic leukemia (ALL), adult T-cell ALL, acute myeloid leukemia (AML), AML with trilineage myelodysplasia, acute promyelocytic leukemia, acute undifferentiated leukemia, anaplastic large-cell lymphoma, chronic lymphocytic leukemia, chronic myeloid leukemia, chronic neutrophilic leukemia, juvenile myelomonocyctic leukemia, mixed lineage leukemia, myeloproliferative disorders, myelodysplastic syndromes, multiple myeloma, and prolymphocytic leukemia.

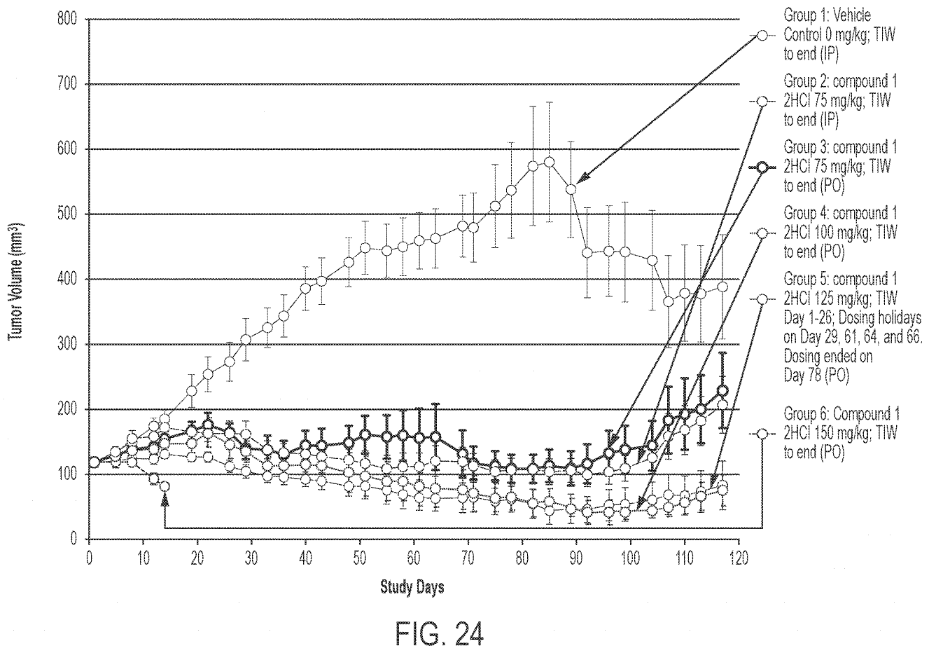

As demonstrated in the Examples, oral formulations of Hsp90 inhibitors as provided herein are effective in reducing tumor burden in animal models of triple negative breast cancer. The oral formulation of Hsp90 inhibitors enabled larger doses to be administered to the subjects without the toxicity that was apparent when such doses were administered by parenteral routes such as intravenous or intraperitoneal administration. The effects of orally formulated Hsp90 inhibitors were observed during the treatment period but also beyond the last administration of the Hsp90 inhibitor. For example, as shown in FIG. 24, tumor burden stayed relatively constant after the last administered dose of the Hsp90 inhibitor in the higher dose groups (100 and 125 mg/kg groups).

Neurodegenerative Disorder

The term “neurodegenerative disorder” refers to a disorder in which progressive loss of neurons occurs either in the peripheral nervous system or in the central nervous system. Examples of neurodegenerative disorders include but are not limited to chronic neurodegenerative diseases such as diabetic peripheral neuropathy, Alzheimer's disease, Pick's disease, diffuse Lewy body disease, progressive supranuclear palsy (Steel-Richardson syndrome), multisystem degeneration (Shy-Drager syndrome), motor neuron diseases including amyotrophic lateral sclerosis (“ALS”), degenerative ataxias, cortical basal degeneration, ALS-Parkinson's-Dementia complex of Guam, subacute sclerosing panencephalitis, Huntington's disease, Parkinson's disease, multiple sclerosis, synucleinopathies, primary progressive aphasia, striatonigral degeneration, Machado-Joseph disease/spinocerebellar ataxia type 3 and olivopontocerebellar degenerations, Gilles De La Tourette's disease, bulbar and pseudobulbar palsy, spinal and spinobulbar muscular atrophy (Kennedy's disease), primary lateral sclerosis, familial spastic paraplegia, Wernicke-Korsakoff's related dementia (alcohol induced dementia), Werdnig-Hoffmann disease, Kugelberg-Welander disease, Tay-Sach's disease, Sandhoff disease, familial spastic disease, Wohifart-Kugelberg-Welander disease, spastic paraparesis, progressive multifocal leukoencephalopathy, and prion diseases (including Creutzfeldt-Jakob, Gerstmann-Straussler-Scheinker disease, Kuru and fatal familial insomnia).

Other conditions also included within the methods of the present disclosure include age-related dementia and other dementias, tauopathies, and conditions with memory loss including vascular dementia, diffuse white matter disease (Binswanger's disease), dementia of endocrine or metabolic origin, dementia of head trauma, chronic traumatic encephalopathy, and diffuse brain damage, dementia pugilistica, and frontal lobe dementia. Also other neurodegenerative disorders resulting from cerebral ischemia or infarction including embolic occlusion and thrombotic occlusion as well as intracranial hemorrhage of any type (including but not limited to epidural, subdural, subarachnoid, and intracerebral), and intracranial and intravertebral lesions (including but not limited to contusion, penetration, shear, compression, and laceration).

Thus, the term “neurodegenerative disorder” also encompasses acute neurodegenerative disorders such as those involving stroke, traumatic brain injury, chronic traumatic encephalopathy, schizophrenia, peripheral nerve damage, hypoglycemia, spinal cord injury, epilepsy, anoxia, and hypoxia.

In certain embodiments, the neurodegenerative disorder is selected from Alzheimer's disease, Parkinson's disease, Huntington disease, amyotrophic lateral sclerosis, complete androgen insensitivity syndrome (CAIS), spinal and bulbar muscular atrophy (SBMA or Kennedy's disease), sporadic frontotemporal dementia with parkinsonism (FTDP), familial FTDP-17 syndromes, age-related memory loss, senility, and age-related dementia. In another embodiment, the neurodegenerative disorder is Alzheimer's disease, also characterized as an amyloidosis. Thus, other embodiments of the disclosure relate to the treatment or prevention of other amyloidosis disorders which share features, including, but not limited to, hereditary cerebral angiopathy, normeuropathic hereditary amyloid, Down's syndrome, macroglobulinemia, secondary familial Mediterranean fever, Muckle-Wells syndrome, multiple myeloma, pancreatic- and cardiac-related amyloidosis, chronic hemodialysis arthropathy, Finnish amyloidosis, and Iowa amyloidosis.

Inflammation (or Inflammatory Conditions)

The Hsp90 inhibitors of this disclosure may be used in the treatment of inflammation (or inflammatory conditions). Examples of inflammatory conditions include cardiovascular diseases and autoimmune diseases.

Non-autoimmune inflammatory disorders are inflammatory disorders that are not autoimmune disorders. Examples include atherosclerosis, myocarditis, myocardial infarction, ischemic stroke, abscess, asthma, some inflammatory bowel diseases, chronic obstructive pulmonary disease (COPD), allergic rhinitis, non-autoimmune vasculitis (e.g. polyarteritis nodosa), age related macular degeneration, alcoholic liver disease, allergy, allergic asthma, anorexia, aneurism, aortic aneurism, atopic dermatitis, cachexia, calcium pyrophosphate dihydrate deposition disease, cardiovascular effects, chronic fatigue syndrome, congestive heart failure, corneal ulceration, enteropathic arthropathy, Felty's syndrome, fever, fibromyalgia syndrome, fibrotic disease, gingivitis, glucocorticoid withdrawal syndrome, gout, hemorrhage, viral (e.g., influenza) infections, chronic viral (e.g., Epstein-Barr, cytomegalovirus, herpes simplex virus) infection, hyperoxic alveolar injury, infectious arthritis, intermittent hydrarthrosis, Lyme disease, meningitis, mycobacterial infection, neovascular glaucoma, osteoarthritis, pelvic inflammatory disease, periodontitis, polymyositis/dermatomyositis, post-ischaemic reperfusion injury, post-radiation asthenia, pulmonary emphysema, pydoderma gangrenosum, relapsing polychondritis, Reiter's syndrome, sepsis syndrome, Still's disease, shock, Sjogren's syndrome, skin inflammatory diseases, stroke, non-autoimmune ulcerative colitis, bursitis, uveitis, osteoporosis, Alzheimer's disease, ataxia telangiectasia, non-autoimmune vasculitis, non-autoimmune arthritis, bone diseases associated with increased bone resorption, ileitis, Barrett's syndrome, inflammatory lung disorders, adult respiratory distress syndrome, and chronic obstructive airway disease, inflammatory disorders of the eye including corneal dystrophy, trachoma, onchocerciasis, sympathetic ophthalmitis and endophthalmitis, chronic inflammatory disorders of the gums such as gingivitis, tuberculosis, leprosy, inflammatory diseases of the kidney including uremic complications, glomerulonephritis and nephrosis, inflammatory disorders of the skin including sclerodermatitis and eczema, inflammatory diseases of the central nervous system, including chronic demyelinating diseases of the nervous system, AIDS-related neurodegeneration and Alzheimer's disease, infectious meningitis, encephalomyelitis, Parkinson's disease, Huntington's disease, amyotrophic lateral sclerosis and viral or autoimmune encephalitis, immune-complex vasculitis, erythematodes, and inflammatory diseases of the heart such as cardiomyopathy, ischemic heart disease, hypercholesterolemia, as well as various other diseases with significant inflammatory components, including preeclampsia, chronic liver failure, septic shock, haemodynamic shock, sepsis syndrome, malaria, diseases involving angiogenesis, skin inflammatory diseases, radiation damage, hyperoxic alveolar injury, periodontal disease, non-insulin dependent diabetes mellitus, and brain and spinal cord trauma.

Cardiovascular Diseases

The Hsp90 inhibitors of this disclosure may be used in the treatment of cardiovascular diseases. Examples of cardiovascular diseases (or conditions) include atherosclerosis, elevated blood pressure, heart failure or a cardiovascular event such as acute coronary syndrome, myocardial infarction, myocardial ischemia, chronic stable angina pectoris, unstable angina pectoris, angioplasty, stroke, transient ischemic attack, claudication(s), or vascular occlusion(s).

Autoimmune Diseases

The Hsp90 inhibitors of this disclosure may be used in the treatment of autoimmune diseases. Examples of autoimmune diseases include but are not limited to multiple sclerosis, inflammatory bowel disease including Crohn's Disease and ulcerative colitis, rheumatoid arthritis, psoriasis, type I diabetes, uveitis, Celiac disease, pernicious anemia, Srojen's syndrome, Hashimoto's thyroiditis, Graves' disease, systemic lupus erythamatosis, acute disseminated encephalomyelitis, Addison's disease, Ankylosing spondylitis, antiphospholipid antibody syndrome, Guillain-Barre syndrome, idiopathic thrombocytopenic purpura, Goodpasture's syndrome, Myasthenia gravis, Pemphigus, giant cell arteritis, aplastic anemia, autoimmune hepatitis, Kawaski's disease, mixed connective tissue disease, Ord throiditis, polyarthritis, primary biliary sclerosis, Reiter's syndrome, Takaysu's arteritis, vitiligo, warm autoimmune hemolytic anemia, Wegener's granulomatosis, Chagas' disease, chronic obstructive pulmonary disease, and sarcoidosis.

### Secondary Therapeutic Agents

The Hsp90 inhibitors of this disclosure may be used in combination with one or more other therapeutic agents, referred to herein as secondary therapeutic agents. The Hsp90 inhibitors and secondary therapeutic agents may have an additive effect or a synergistic (i.e., greater than additive) effect on the targeted indication.

Examples of secondary therapeutic agents include angiogenesis inhibitors, pro-apoptotic agents, cell cycle arrest agents, kinase inhibitors, AKT inhibitors, BTK inhibitors, Bcl2 inhibitors, SYK inhibitors, CD40 inhibitors, CD28 pathway inhibitors, MHC class II inhibitors, PI3K inhibitors, mTOR inhibitors, JAK inhibitors, IKK inhibitors, Raf inhibitors, SRC inhibitors, phosphodiesterase inhibitors, ERK-MAPK pathway inhibitors, and the like.

Examples of AKT inhibitors include PF-04691502, Triciribine phosphate (NSC-280594), A-674563, CCT128930, AT7867, PHT-427, GSK690693, MK-2206 dihydrochloride.

Examples of BTK inhibitors include PCI-32765.

Examples of Bcl2 inhibitors include ABT-737, Obatoclax (GX15-070), ABT-263.

TW-37 Examples of SYK inhibitors include R-406, R406, R935788 (Fostamatinib disodium).

Examples of CD40 inhibitors include SGN-40 (anti-huCD40 mAb).

Examples of inhibitors of the CD28 pathway include abatacept, belatacept, blinatumomab, muromonab-CD3, visilizumab.

Examples of inhibitors of major histocompatibility complex, class II include apolizumab.

Examples of PI3K inhibitors include 2-(1H-indazol-4-yl)-6-(4-methanesulfonylpiperazin-1-ylmethyl)-4-morpholin-4-ylthieno(3,2-d)pyrimidine, BKM120, NVP-BEZ235, PX-866, SF 1126, XL147.

Example of mTOR inhibitors include deforolimus, everolimus, NVP-BEZ235, OSI-027, tacrolimus, temsirolimus, Ku-0063794, WYE-354, PP242, OSI-027, GSK2126458, WAY-600, WYE-125132.

Examples of JAK inhibitors include Tofacitinib citrate (CP-690550), AT9283, AG-490, INCBO 18424 (Ruxolitinib), AZD1480, LY2784544, NVP-BSK805, TGI 01209, TG-101348.

Examples of IkK inhibitors include SC-514, PF 184.

Examples of inhibitors of Raf include sorafenib, vemurafenib, GDC-0879, PLX-4720, PLX4032 (Vemura/enib), NVP-BHG712, SB590885, AZ628, ZM 336372.

Examples of inhibitors of SRC include AZM-475271, dasatinib, saracatinib.

Examples of inhibitors of phosphodiesterases include aminophylline, anagrelide, arofylline, caffeine, cilomilast, dipyridamole, dyphylline, L 869298, L-826,141, milrinone, nitroglycerin, pentoxifylline, roflumilast, rolipram, tetomilast, theophylline, tolbutamide, amrinone, anagrelide, arofylline, caffeine, cilomilast, L 869298, L-826,141, milrinone, pentoxifylline, roflumilast, rolipram, tetomilast.

Other secondary therapeutic agents that can be used in combination with the Hsp90 inhibitors of this disclosure include AQ4N, becatecarin, BN 80927, CPI-0004Na, daunorubicin, dexrazoxane, doxorubicin, elsamitrucin, epirubicin, etoposide, gatifloxacin, gemifloxacin, mitoxantrone, nalidixic acid, nemorubicin, norfloxacin, novobiocin, pixantrone, tafluposide, TAS-103, tirapazamine, valrubicin, XK469, BI2536.

Still other secondary therapeutic agents are nucleoside analogs. Examples include (1) deoxyadenosine analogues such as didanosine (ddl) and vidarabine; (2) adenosine analogues such as BCX4430; (3) deoxycytidine analogues such as cytarabine, gemcitabine, emtricitabine (FTC), lamivudine (3TC), and zalcitabine (ddC); (4) guanosine and deoxyguanosine analogues such as abacavir, acyclovir, and entecavir; (5) thymidine and deoxythymidine analogues such as stavudine (d4T), telbivudine, zidovudine (azidothymidine, or AZT); and (6) deoxyuridine analogues such as idoxuridine and trifluridine.

Other secondary therapeutic agents include taxanes such as paclitaxel, dicetaxel, cabazitaxel. Other secondary therapeutic agents include inhibitors of other heatshock proteins such as of Hsp70, Hsp60, and Hsp26.

Still other secondary therapeutic agents that can be used in combination with the Hsp90 inhibitors of this disclosure are disclosed in published PCT Application No. WO2012/149493, the entire disclosure of which as it relates to such secondary therapeutic agents and classes thereof is incorporated by reference herein.

The Hsp90 inhibitors and the secondary therapeutic agents may be co-administered. Co-administered includes administering substantially simultaneously, concomitantly, sequentially or adjunctively. The Hsp90 inhibitors and the secondary therapeutic agents may be administered at different times. For example, the Hsp90 inhibitors may be administered before or after the secondary therapeutic agent including one or more hours before, one or more day before, or one or more week before the secondary therapeutic agents. One or more secondary therapeutic agents may be used. Each of the therapeutic agents may be administered at their predetermined optimal frequency and dose. In some instances, the Hsp90 inhibitors and the secondary therapeutic agents are administered in combination in a therapeutically effective amount.

As an example, this disclosure provides a method of treating a subject having a cancer and the method comprises co-administering to the subject (a) an inhibitor of Hsp90 and (b) an inhibitor of Btk. Another example provided herein is a method of treating a subject having a cancer comprising co-administering to the subject (a) an inhibitor of Hsp90 and (b) an inhibitor of Syk. In such methods the cancer may be a lymphoma. Yet another example provided herein is a method of treating a subject having a chronic myelogenous leukemia (CML) and the method comprises co-administering to the subject (a) an inhibitor of Hsp90 and (b) an inhibitor of any of mTOR, IKK, MEK, NF.kappa.B, STAT3, STAT5A, STAT5B, Raf-1, bcr-abl, CARM1, CAMKII, or c-MYC.

### Examples

### Example 1

This Example examined the anti-tumor activity of Compound 1 provided in a dihydrochloride (2HCl) form as a single agent in the MDA-MB-468 triple negative breast tumor xenograft model. In particular, the efficacy of intraperitoneal (IP) and oral administration (PO) of Compound 1 dihydrochloride (2HCl) was compared.

### Materials and Methods

The animals used in this study were Nu/Nu (NU-Foxn1^{nu}) (athymic nude) physiologically normal female mice supplied by Charles River. At the time of inoculation, the age of the animals was 5-8 weeks. Sixty total animals were used and animals were not replaced during the course of this study. Mice were identified with a transponder. The animals were housed in individually ventilated microisolator cages and allowed to acclimate for at least 5-7 days. The animals were maintained under pathogen-free conditions and given Teklad Global Diet® 2920x irradiated pellets for food and autoclaved water ad libitum.

Compound ldihydrochloride (2HCl) was provided as a crystalline powder and stored at 2-8° C. protected from light. The administered form of Compound 1 2HCl was a clear solution. For intraperitoneal administration, Compound 1 2HCl was reconstituted in PBS. For oral administration, Compound 1 2HCl was reconstituted in 0.5% Methylcellulose (MC) in water. The salt: base ratio was 1.14:1 (i.e., to obtain 100 mg of Compound 1 free base, 114 mg of Compound 1 dihydrochloride salt was weighed out). Dose levels of Compound 1 were based on the free base, not the salt. Compound 1 2HCl in administered form was prepared fresh immediately prior to use.

To form the xenografts, 1×10⁷ MDA-MB-468 cells suspended in 0.1 ml of 50% Matrigel/50% Media (1:1) were injected into the mammary fat pad of each mouse. Treatment was initiated when the mean tumor size reached 100-150 mm³ and the day of treatment initiation was designated as Day 1. Subcutaneous tumor size was calculated as tumor volume (mm³)=(a×b²/2), where ‘b’ is the smallest diameter and ‘a’ is the largest diameter.

Animals were randomized using random equilibration of tumor volume into one of six study groups, as shown in Table 22 (Groups 1-6), with 10 animals in each group.

TABLE 22
Study Groupings

| | | Vehicle | | |
|---|---|---|---|---|
| | | Control | In PBS | In MC |
| | | (TIW to | (TIW to | (TIW to |
| Group | N | End) | End) | End) |
| Vehicle Control (IP) | 10 | x | | |
| Compound 1 2HC175 mg/kg (IP) | 10 | | x | |
| Compound 1 2HC175 mg/kg (PO) | 10 | | | x |
| Compound 1 2HC1 100 mg/kg (PO) | 10 | | | x |
| Compound 1 2HC1 125 mg/kg (PO) | 10 | | | x |
| Compound 1 2HC1 150 mg/kg (PO) | 10 | | | x |

Group 1 was administered vehicle control alone (without Compound 1 2HCl) intraperitoneally (IP) three times weekly (TIW) until the end of the study. PBS was used as the vehicle control and was administered at a volume of 10 mL/kg.

Groups 2-6 were administered Compound 1 2HCl at a volume of 10 mL/kg three times weekly (TIW) until the end of the study with the doses as described next.

Group 2 received 75 mg/kg Compound 1 2HCl via intraperitoneal administration.

Group 3 received 75 mg/kg Compound 1 2HCl via oral administration (PO). Group 4 received 100 mg/kg Compound 1 2HCl via oral administration. Group 5 received 125 mg/kg Compound 1 2HCl via oral administration. Group 6 received 150 mg/kg Compound 1 2HCl via oral administration. Oral gavage was used for oral administration.

Tumor volume and body weight were measured twice weekly with gross observations daily. Individual mice were euthanized when tumor volume was ≥1500 mm³. Mice that did not reach the endpoint tumor volume of ≥1500 mm³ will be euthanized on Day 90.

For data analysis, simple statistics (ANOVA) will be conducted on tumor volumes to verify significance of treatment groups relative to control. Growth curves will be constructed and percent tumor growth inhibition (TGI) will be calculated as a means to assess the effect of the single-agent therapy regimens. Kaplan-Meier curves will be constructed upon the tumor reaching volume endpoint. Percent mouse weight change graphs will be used to evaluate dose tolerance of the therapies.

### Results

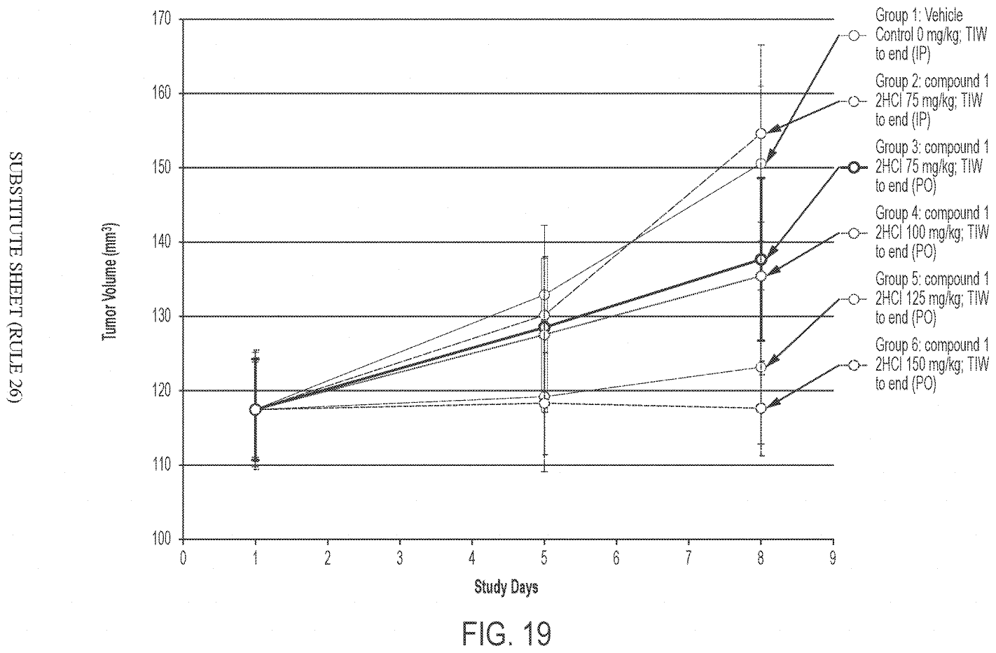

As demonstrated in FIG. 19, oral administration of Compound 1 2HCl was as efficacious in inhibiting tumor growth of MDA-MB-468 breast tumor xenografts in mice as intraperitoneal administration of Compound 1 2HCl at same dose levels (75 mg/kg). Tumor volume was measured over the course of 8 days (Study Days 1-8) to assess the effect of each treatment on xenograft growth. Tumor volume was measured for animals receiving intraperitoneal administration of vehicle control (Group 1) to determine tumor growth in the absence of Compound 1 2HCl. As anticipated, tumors continued to grow in animals receiving PBS (Group 1). Intraperitoneal administration of 75 mg/kg Compound 1 2HCl did not inhibit tumor growth in animals (Group 2). Notably, when the same dose of 75 mg/kg Compound 1 2HCl was administered orally (Group 3), tumor growth was reduced (compare Group 3 tumor volume with Group 2 tumor volume at Day 8 in FIG. 19). Inhibition of tumor growth was also observed in Group 4 treated with 100 mg/kg Compound 1 2HCl via oral administration compared to Group 1.

A dose-dependent response was detected with increasing doses of orally administered Compound 1 2HCl (Groups 3-5). For example, the greatest suppression of tumor growth was detected with the highest doses of orally administered Compound 1 2HCl (125 mg/kg dose in Group 5 and 150 mg/kg dose in Group 6).

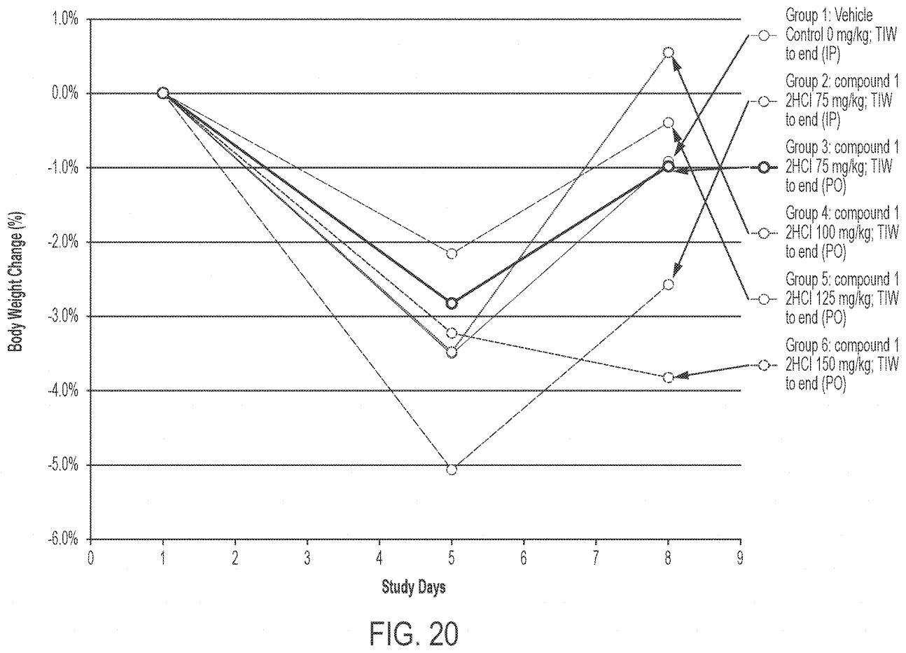

As shown in FIG. 20, the tumor inhibition detected with oral administration of Compound 1 2HCl was likely not associated with treatment toxicity (dose tolerance). Except at the highest dose of orally administered Compound 1 2HCl tested (Group 6), animals receiving oral administration of Compound 1 2HCl (Groups 3-5) had similar body weight change percentages over the course of the study as control Group 1. Notably, intraperitoneal administration of 75 mg/kg Compound 1 2HCl (Group 2) induced a greater decrease in body weight compared to Groups 1-5 at Day 5 and at Day 8.

This Example demonstrates that oral administration of Compound 1 2HCl at tolerable doses was more efficacious in inhibiting tumor growth compared to intraperitoneal administration of Compound 1 2HCl over the 8 day period studied. The treatment of these mice continued for longer periods of time as reported in Examples 2 and 3.

### Example 2

This Example examined the anti-tumor activity of Compound 1 provided in a dihydrochloride (2HCl) form as a single agent in the MDA-MB-468 triple negative breast tumor xenograft model over a longer period of treatment (36 days). The efficacy of intraperitoneal (IP) and oral administration (PO) of Compound 1 dihydrochloride (2HCl) was compared.

### Materials and Methods

The Materials and Methods used were the same as discussed above for Example 1, except for Group 5 and Group 6. For Group 5, there was a dosing holiday on Day 29 of treatment. Mice in Group 5 were administered Compound 1 2HCl at a volume of 10 mL/kg three times weekly (TIW) with 125 mg/kg Compound 1 2HCl via oral administration on days 1 through 26 of the study, given a dosing holiday on Day 29, and dosing was resumed on Day 31 until the end of the study. Data were only available for Days 1-14 of the study for Group 6.

### Results

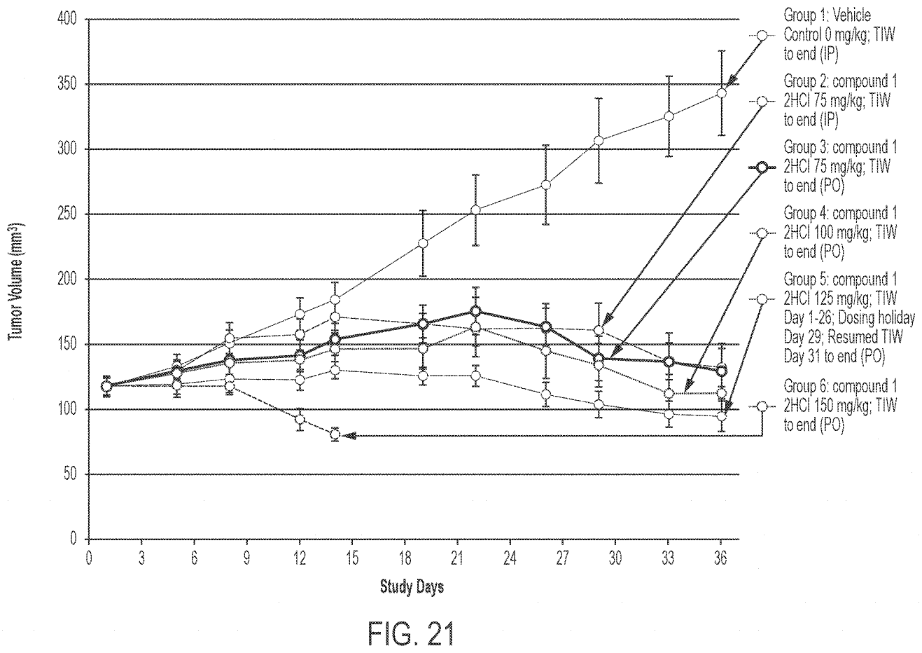

As demonstrated in FIG. 21, oral administration of Compound 1 2HCl was at least as efficacious in inhibiting tumor growth of MDA-MB-468 breast tumor xenografts in mice as intraperitoneal administration of Compound 1 2HCl over the study period. Tumor volume was measured over the course of 36 days (Study Days 1-36) to assess the effect of each treatment on xenograft growth. Tumor volume was measured for animals receiving intraperitoneal administration of vehicle control (Group 1) to determine tumor growth in the absence of Compound 1 2HCl. As anticipated, tumors continued to grow in animals receiving PBS (Group 1) over the 36 days of the study. Oral administration of 75 mg/kg Compound 1 2HCl inhibited tumor growth slightly more than intraperitoneal administration of the same dose of Compound 1 2HCl over the first 14 days of treatment (see Groups 2 and 3 at Day 14 in FIG. 21). A dose-dependent response was detected with increasing doses of orally administered Compound 1 2HCl (Groups 3-5). At Day 36, tumor inhibition was observed in mice receiving 75 mg/kg Compound 1 2HCl by intraperitoneal administration or oral administration. Tumor inhibition was also observed in mice receiving 100 mg/kg and 125 mg/kg Compound 1 2HCl at Day 36. Oral administration of 125 mg/kg Compound 1 2HCl over the 36 day period also caused tumor regression.

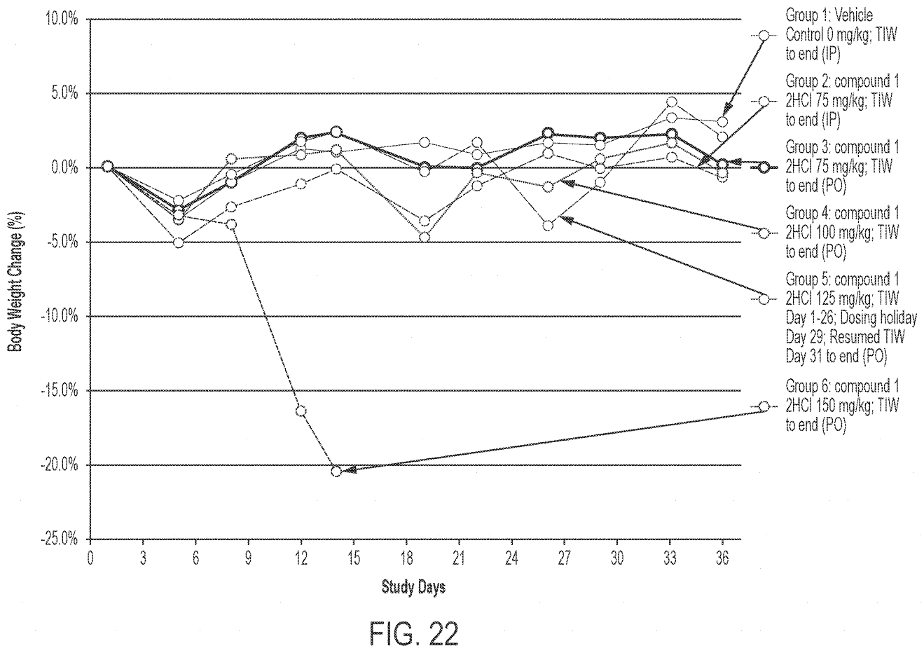

As shown in FIG. 22, the tumor inhibition detected with oral administration of Compound 1 2HCl was likely not associated with treatment toxicity (dose tolerance). Animals receiving oral administration of Compound 1 2HCl (Groups 3-5) had similar body weight change percentages over the course of the study as control Group 1.

This Example demonstrates that oral administration of Compound 1 2HCl at tolerable doses was as or more efficacious in inhibiting tumor growth as intraperitoneal administration of Compound 1 2HCl. The treatment of these mice continued for longer periods of time as reported in Example 3.

### Example 3

This Example examined the anti-tumor activity of Compound 1 provided in a dihydrochloride (2HCl) form as a single agent in the MDA-MB-468 triple negative breast tumor xenograft model over a longer period of treatment (89 days). The efficacy of intraperitoneal (IP) and oral administration (PO) of Compound 1 dihydrochloride (2HCl) was compared.

### Materials and Methods

The Materials and Methods used were the same as discussed above for Example 2, except for Group 5 (125 mg/kg PO). Mice in Group 5 were administered Compound 1 2HCl at a volume of 10 mL/kg three times weekly (TIW) with 125 mg/kg Compound 1 2HCl via oral administration, but there were dosing holidays on Day 29, 61, 64, and 66 and dosing ended on Day 78.

### Results

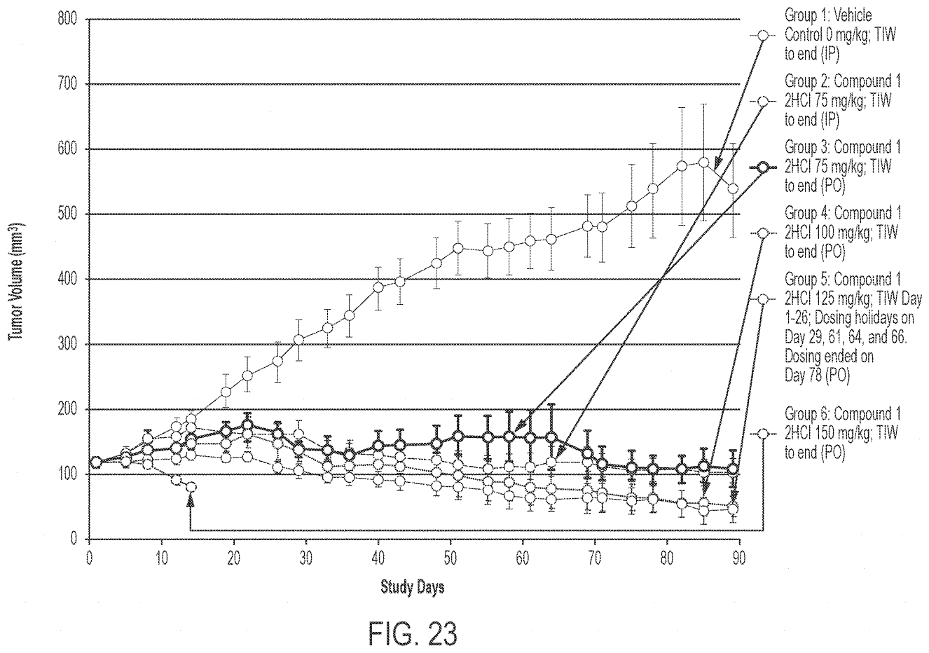

As demonstrated in FIG. 23, oral administration of Compound 1 2HCl was as or more efficacious in inhibiting tumor growth of MDA-MB-468 breast tumor xenografts in mice as intraperitoneal administration of Compound 1 2HCl. Tumor inhibition and/or regression were observed with doses of orally administered Compound 1 2HCl ranging from 75 mg/kg through to 125 mg/kg. Tumor volume was measured over the course of 89 days (Study Days 1-89) to assess the effect of each treatment on xenograft growth. Tumor volume was measured for animals receiving intraperitoneal administration of vehicle control to determine tumor growth in the absence of Compound 1 2HCl. As anticipated, tumors continued to grow in animals receiving PBS (control) over the 89 days of the study. Tumor growth was inhibited in mice receiving intraperitoneal administration of 75 mg/kg Compound 1 2HCl and in mice receiving oral administration of 75 mg/kg Compound 1 2HCl. Mean tumor volume in mice receiving 75 mg/kg Compound 1 2HCl either orally or intraperitoneally was about 20% of the mean tumor volume in control mice receiving vehicle alone, at Day 89. Higher doses (100 mg/kg and 125 mg/kg) of orally administered Compound 1 2HCl were tumor regressive. Mean tumor volume in mice receiving 100 mg/kg and 125 mg/kg Compound 1 2HCl orally was about 50% of the mean tumor volume in mice receiving 75 mg/kg Compound 1 2HCl either orally or intraperitoneally, at Day 89.

This Example demonstrates that oral administration of Compound 1 2HCl is as efficacious or more efficacious than intraperitoneal administration of Compound 1 2HCl. Higher doses of Compound 1 2HCl are better tolerated when administered orally than when administered intraperitoneally (partial data shown). These higher oral doses are associated with tumor regression. Thus, these data evidence the ability to orally administer, over a 3 month period of time, Compound 1 2HCl, at doses that cause tumor growth inhibition and, for some doses, tumor regression.

### Example 4

This Example examined the antitumor effect of Compound 1 provided in a dihydrochloride (2HCl) form as a single agent in the MDA-MB-468 triple negative breast tumor xenograft model after treatment was stopped. The efficacy of intraperitoneal (IP) and oral administration (PO) of Compound 1 dihydrochloride (2HCl) was compared.

### Materials and Methods

The Materials and Methods used were the same as discussed above for Example 3, except for the lengths of treatment for Groups 1-4. Treatment for Groups 1-4 was stopped on Day 103. Tumor growth and body weight were measured twice weekly with gross observations daily for Groups 1-5 until Day 117.

### Results

As demonstrated in FIG. 24, oral administration of Compound 1 2HCl was more efficacious at inhibiting tumor regrowth at higher doses compared to intraperitoneal administration of the maximum tolerated dose of Compound 1 2HCl. Tumor inhibition was observed with orally administered Compound 1 2HCl at the 100 mg/kg dose (Group 4) and at the 125 mg/kg dose (Group 5) even after the end of treatment, whereas tumor regrowth was observed with the maximum tolerated dose of intraperitoneally administered Compound 1 2HCl (75 mg/kg, Group 2). As described in the Materials and Methods section above, treatment for Groups 1-4 was stopped on Day 103 and treatment for Group 5 was stopped on Day 78 (with dosing holidays on Days 29, 61, 64 and 66). Treatment for Group 6 was stopped on Day 14 due to toxicity. Tumor volume was measured over the course of 117 days (Study Days 1-117) to assess the effect of Compound 1 2HCl on xenograft growth during each treatment and after each treatment. As anticipated, tumor volume remained high (in the range of about 365-429 mm³) in animals receiving PBS (control) between days 104 and 117, after PBS treatment was stopped. Tumor regrowth was observed after treatment with 75 mg/kg orally administered and 75 mg/kg intraperitoneally administered Compound 1 2HCl was stopped. Mean tumor volume in mice receiving 75 mg/kg either orally or intraperitoneally on Day 117 was about 1.7-1.9 times higher than the mean tumor volume in the same mice at Day 1. Notably, the maximum tolerated dose of Compound 1 2HCl by intraperitoneal administration is 75 mg/kg. In contrast, inhibition of tumor regrowth was observed at higher doses (100 mg/kg and 125 mg/kg) of orally administered Compound 1 2HCl even after treatment was stopped. Mean tumor volume in mice receiving 100 mg/kg and 125 mg/kg Compound 1 2HCl orally was about 63% and 70% respectively of the mean tumor volume in the same mice at day 1.

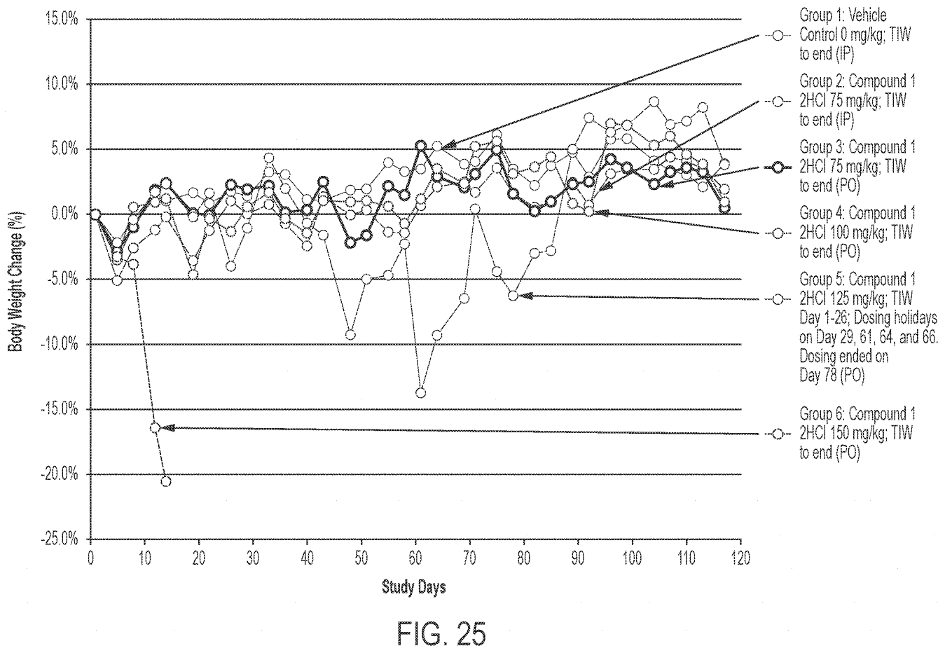

As shown in FIG. 25, oral administration of a higher dose of Compound 1 2HCl (e.g., the 100 mg/kg dose) has minimal effects on body weight, similar to the maximum tolerated dose of intraperitoneally administered Compound 1 2HCl (75 mg/kg IP). Drug dosing holidays (e.g., on Days 64 and 66 and the end of treatment on day 78) rescued the effect of 125 mg/kg orally administered Compound 1 2HCl on body weight (FIG. 25) with minimal effects on antitumor activity (FIG. 24).

This Example demonstrates that oral administration of Compound 1 2HCl can continue to be effective at higher doses of Compound 1 2HCl, even with drug dosing holidays. In contrast, tumor regrowth was observed with the maximum tolerated dose of intraperitoneally administered Compound 1 2HCl after drug dosing was stopped. Thus, these data show that Compound 1 2HCl may be administered over a 4 month period of time at higher oral doses that prevent tumor regrowth following a drug dosing holiday.

### Example 5

This Example examined the plasma pharmacokinetics (PK) of Compound 1 provided in a dihydrochloride (2HCl) form and Compound 2 provided in a free base form following single administration in Sprague Dawley Rats. In particular, the bioavailability following oral administration (PO) of Compound 1 dihydrochloride (2HCl) in ORA-Plus® solution, oral administration (PO) of Compound 1 2HCl dissolved in 0.5% aqueous methylcellulose, and intravenous administration (IV) of Compound 1 2HCl dissolved in 0.9% Saline were compared. For Compound 2, the bioavailability following oral administration of Compound 2 free base suspended in ORA-Plus® drinking solution, oral administration of Compound 2 free base suspended into 30% Captisol® in 60 mM citrate buffer, and intravenous administration of Compound 2 free base dissolved into 15% Captisol® in 5 mM citrate buffer were compared.

### Materials and Methods

The animals used in this study were female Sprague Dawley Rats physiologically normal. At the time of receipt, mice were 200-225 g in weight. Three rat deaths were reported in the group receiving 30% Captisol® in 60 mM citrate buffer. Ninety-four total animals were observed thereafter. The parenteral administration is performed by tail vein injection.

Compound 2 was provided in free base form and stored at −20° C., protected from light. Compound 2 was formulated in dosage form immediately prior to use. For oral administration of Compound 2 in ORA-Plus® drinking solution, Compound 2 was suspended in drinking solution ORA-Plus® (Perrigo; Minneapolis, Minn.). First, a mortar and pestle were used to smooth out the Compound 2 powder, then a small amount of ORA-Plus® was added, and next, the mixture was triturated to a thick, smooth paste. The remainder of the ORA-Plus® was added by geometric dilution. The Compound 2 free base and ORA-Plus® mixture was dispensed in a tight, light resistant amber bottle with appropriate labeling. This mixture was shaken well before using, protected from light and kept refrigerated if dosing was delayed. For oral administration of Compound 2 in citric acid buffer with Captisol®, Compound 2 free base powder was dissolved or suspended into 30% Captisol® (Cydex Pharmaceuticals; Lawrence, Kans.) in 60 mM citrate buffer (pH˜4.2) (citric acid and sodium citrate dehydrate (Sigma-Aldrich; St. Louis Mo.)) in sterile water) to each group's working concentration. Formulation for treatment groups 6, 7, and 8 (see Table 23 below) were a slightly hazy suspension. Formulation for group 5 (see Table 23 below) was a clear solution. A magnetic stir-bar was used to mix dosing solution, followed by sonication. For intravenous administration, Compound 2 free base powder was dissolved into 15% Captisol® in 5 mM citrate buffer (pH˜4.2) to each group's working concentration. A magnetic stirbar was used to mix dosing solution, followed by sonication. IV dosing solution of Compound 2 free base was filtered with a 0.2 m PVDF filter (Pall Life Sciences; Port Washington, N.Y.) prior to administration.

Compound 1 dihydrochloride (2HCl) was provided as a crystalline powder and stored at 4 C protected from light. The administered form of Compound 1 2HCl was a clear solution. For oral administration of Compound 1 2HCl suspended in ORA-Plus® drinking solution, a mortar and pestle were used to smooth out the powder and a small amount of ORA-Plus® was added and the mixture was triturated to a think, smooth paste. The remainder of the ORA-Plus® was added by geometric dilution. The Compound 1 2HCl and ORA-Plus® mixture was dispensed in a tight, light resistant amber bottle with appropriate labeling. This mixture was shaken well before using, protected from light and kept refrigerated if dosing was delayed. For oral administration of Compound 1 2HCl in methylcellulose, Compound 1 2HCl was dissolved in 0.5% aqueous methylcellulose (0.375 g methylcellulose (Sigma-Aldrich) in 75 mL sterile water) by gentle vortex. For intravenous administration of Compound 1 2HCl, Compound 1 2HCl was dissolved in 0.9% Saline (Baxter Healthcare; Deerfield, Ill.) by gentle vortex. The salt:base ratio is 1.14:1 (a correction factor of 1.14 was applied to the Compound 1 dihydrochloride salt to obtain the correct amount of Compound 1 free base). Dose levels of Compound 1 were based on the free base, not the salt. Compound 1 2HCl in administered form was prepared fresh immediately prior to use.

Animals were randomized using random equilibration of body weights on Day 1 into one of 19 study groups, as shown in Table 23 (Groups 1-19), with 5 animals in each group, except for the 4 animals in Group 19. Body weights were collected Days 1, 2, 3, and/or 4 to accommodate data collection of staggered groups. Gross observations of body weight were noted during the course of the study. Treatment initiation was staggered by group to accommodate collections, resulting in multiple treatment initiation days. Groups with like compound/vehicle/administration route were performed together when possible. Therefore, treatment was initated on Day 1, 2, 3 or 4. The study endpoint followed the final collected timepoint for each group.

TABLE 23
Study Groupings

| | | | | Compound 2 | Compound 2 | | | |
|---|---|---|---|---|---|---|---|---|
| | | | Compound 2 | (Citric Acid | (Citric Acid | Compound 1 | Compound 1 | Compound 1 |
| | | | (ORA-Plus ®) | Buffer-PO) | Buffer-IV) | (ORA-Plus ®) | (MC-PO) | (Saline-IV) |
| Group | | N | [Single Dose] | [Single Dose] | [Single Dose] | [Single Dose] | [Single Dose] | [Single Dose] |
| 1. | Compound 2 in ORA- | 5 | X | | | | | |
| | Plus ® 24 mg/kg (PO) | | | | | | | |
| 2. | Compound 2 in ORA- | 5 | X | | | | | |
| | Plus ® 36 mg/kg (PO) | | | | | | | |
| 3. | Compound 2 in ORA- | 5 | X | | | | | |
| | Plus ® 48 mg/kg (PO) | | | | | | | |
| 4. | Compound 2 in ORA- | 5 | X | | | | | |
| | Plus ® 60 mg/kg (PO) | | | | | | | |
| 5. | Compound 2 in Citric | 5 | | X | | | | |
| | Acid Buffer 24 mg/kg (PO) | | | | | | | |
| 6. | Compound 2 in Citric | 5 | | X | | | | |
| | Acid Buffer 36 mg/kg (PO) | | | | | | | |
| 7. | Compound 2 in Citric | 5 | | X | | | | |
| | Acid Buffer 48 mg/kg (PO) | | | | | | | |
| 8. | Compound 2 in Citric | 5 | | X | | | | |
| | Acid Buffer 60 mg/kg (PO) | | | | | | | |
| 9. | Compound 2 in Citric | 5 | | | X | | | |
| | Acid Buffer 12 mg/kg (IV) | | | | | | | |
| 10. | Compound 2 in Citric | 5 | | | X | | | |
| | Acid Buffer 24 mg/kg (IV) | | | | | | | |
| 11. | Compound 1 in ORA-Plus ® | 5 | | | | X | | |
| | 24 mg/kg (PO) | | | | | | | |
| 12. | Compound 1 in ORA-Plus ® | 5 | | | | X | | |
| | 36 mg/kg (PO) | | | | | | | |
| 13. | Compound 1 in ORA-Plus ® | 5 | | | | X | | |
| | 48 mg/kg (PO) | | | | | | | |
| 14. | Compound 1 in ORA-Plus ® | 5 | | | | X | | |
| | 60 mg/kg (PO) | | | | | | | |
| 15. | Compound 1-MC | 5 | | | | | X | |
| | 36 mg/kg (PO) | | | | | | | |
| 16. | Compound 1 -MC | 5 | | | | | X | |
| | 48 mg/kg (PO) | | | | | | | |
| 17. | Compound 1 -MC | 5 | | | | | X | |
| | 60 mg/kg (PO) | | | | | | | |
| 18. | Compound 1-Saline | 5 | | | | | | X |
| | 12 mg/kg (IV) | | | | | | | |
| 19. | Compound 1-Saline | 4 | | | | | | X |
| | 24 mg/kg (IV) | | | | | | | |

Groups 1-8 received a single dose of Compound 2 free base at a volume of 10 mL/kg by oral gavage. Groups 1-4 received a dose of Compound 2 free base in ORA-Plus® drinking solution as indicated in Table 23. Groups 5-8 received a dose of Compound 2 free base in 60 mM Citric Acid Buffer and 30% Captisol® as indicated in Table 23.

Groups 9-10 received a single slow bolus dose of Compound 2 free base at a volume of 10 mL/kg via intravenous tail vein injection. Compound 2 free base was dissolved in 5 mM citric acid buffer and 15% Captisol® to treat Groups 9-10 as indicated in Table 23.

Groups 11-17 received a single dose of Compound 1 2HCl at a volume of 10 mL/kg by oral gavage. Groups 11-14 received a dose of Compound 1 2HCl in ORA-Plus® drinking solution as indicated in Table 23. Groups 15-17 received a single dose of Compound 1 2HCl in 0.5% methylcellulose as indicated in Table 23.

Groups 18-19 received a single slow bolus dose of Compound 1 2HCl at a volume of 10 mL/kg via intravenous tail vein injection. Compound 1 was dissolved in 0.9% saline to treat Groups 18-19 as indicated in Table 23.

Whole Blood was collected from all rats in all groups via jugular vein cannulas pre-dose (T=0), and at 0.25, 0.5, 1, 2, 4, and 6 hours post dose. Blood was placed in li-heparin microtainers (Greiner Bio-one; Kremsmunster, Austria, and Becton, Dickinson & Co; Franklin Lakes, N.J.), centrifuged at 4° C., and processed for plasma. Plasma was removed and placed into a cryovial (Thermo Scientific; Rochester, N.Y.), snap frozen in liquid nitrogen, and stored at −80° C. A sufficient amount of blood was collected from all rats to yield enough plasma for PK analysis.

Samples were analyzed for levels of Compound 2 and Compound 1 by LC-MS/MS.

### Standards

Compound 2 and Compound 1 were provided and internal standard was weighed out for preparation of stocks solutions in DMSO. These solutions were used to spike into plasma for preparation of appropriate standard curves.

### Data Collection

MassLynx software (Waters corp.): Raw data generated.

### Methods: LCMS Analysis and Pharmacokinetic Analysis

Bioanalytical Methods-Compound 2 & Compound 1: Plasma samples were processed for extraction of compounds using protein precipitation and centrifugation. Supernatant from samples were then analyzed against standard calibrators similarly prepared in blank plasma, using a Xevo-TQS mass spectrometer coupled to Acquity UPLC system. Separation was conducted using the appropriate analytical column with analytes monitored in MRM mode. Assessment of linearity, accuracy and precision was made before sample analysis. In brief, calibration curves were calculated by MassLynx software and linearity was determined by comparing the correlation coefficient (r2>0.99) and error between theoretical and back-calculated concentrations of calibration standard samples (<15%, for LLOQ<20%). Calibration curve was used to calculate concentration of quality control samples by interpolation and accuracy assessed.

### Pharmacokinetic Analysis

Calculated concentrations per time points were used for noncompartmental pharmacokinetic analysis using Phoenix WinNonLin software (v. 6.4). Parameters such as maximal concentration achieved (Cₘₐₓ), time to Cₘₐₓ (Tₘₐₓ), area under the curve (AUC) were reported. Calculations for half-life (t½), volume of distribution and clearance were not possible for all groups and therefore were excluded from the summary tables.

### Results

As shown in Table 24, although intravenous administration resulted in higher bioavailability (e.g., higher Cₘₐₓ and higher AUC₀₋ₗₐₛₜ) of Compound 2 free base compared to oral administration of Compound 2 free base at the lower dose of 24 mg/kg, bioavailability of orally administered Compound 2 free base could be increased by using higher oral doses (36 mg/kg, 48 mg/kg or 60 mg/kg). This trend was observed regardless of whether Compound 2 free base was dissolved in ORA-Plus® drinking solution or in citric acid buffer and Captisol®. The mean AUC_{0_last} for higher oral doses of Compound 2 free base was about 1.5 to about 5.3 times higher than the mean AUC₀₋ₗₐₛₜ for the 24 mg/kg oral dose of Compound 2 free base in either vehicle (Groups 2-4 compared to Group 1 in Table 24 and Groups 6-8 compared to Group 5 in Table 24). Furthermore, the mean AUC₀₋ₗₐₛₜ for some of the higher oral doses is comparable to the mean AUC₀₋ₗₐₛₜ for the maximum tolerated dose of intravenously administered Compound 2 free base (24 mg/kg IV) (compare, for example, Group 3 with Group 10 and Group 7 with Group 10 in Table 24).

While the maximum tolerated dose of intravenously administered Compound 2 free base was 24 mg/kg, higher oral doses of Compound 2 free base could be used with minimal effects on body weight and limited toxicity (data not shown). This reduction in toxicity at higher doses of orally administered Compound 2 compared to intravenously administered Compound 2 free base may be due to the higher Tₘₐₓ and lower Cₘₐₓ observed at all oral doses compared to intravenous administration (Table 24). A higher Tₘₐₓ indicates that there was a more gradual increase in serum concentrations of Compound 2 free base with oral administration compared to intravenous administration. Furthermore, the observed maximum serum concentration (Cₘₐₓ) of orally administered Compound 2 free base was lower than intravenous administration, which may limit toxicity.

Except for the lowest orally administered dose, the bioavailability as measured by Cₘₐₓand AUC_{0_last} were comparable for Compound 2 free base prepared in ORA-Plus® drinking solution and for Compound 2 free base prepared in citrate buffer and Captisol® (Table 26).

As shown in Table 25, although intravenous administration resulted in higher bioavailability (e.g., higher Cₘₐₓ and higher AUC₀₋ₗₐₛₜ) of Compound 1 2HCl compared to the bioavailability at lower oral doses (24 mg/kg or 36 mg/kg), bioavailability of orally administered Compound 1 2HCl could be increased by using higher oral doses (48 mg/kg or 60 mg/kg). This trend was observed regardless of whether Compound 1 2HCl was dissolved in ORA-Plus® drinking solution or in methylcellulose in water. Mean AUC₀₋ₗₐₛₜ for higher oral doses of Compound 1 2HCl (48 mg/kg or 60 mg/kg) was about 1.5 to about 2.6 times higher than the mean AUC₀₋ₗₐₛₜ for lower doses of Compound 1 2HCl (24 mg/kg or 36 mg/kg). Furthermore, the mean AUC₀₋ₗₐₛₜ for some of the higher oral doses is comparable to the mean AUC₀₋ₗₐₛₜ for the maximum tolerated dose of intravenously administered Compound 1 2HCl (24 mg/kg IV) (see, e.g., Groups 13 and 14 compared to Group 19 and Groups 16-17 compared to Group 19 in Table 25). A comparison of PK parameters of oral formulations of Compound 1 2HCl relative to the intravenous dose at 24 mg/kg is provided in Table 28.

The bioavailability as measured by Cₘₐₓ and AUC_{0_last} were comparable for Compound 1 2HCl prepared in ORA-Plus® drinking solution and for Compound 1 2HCl prepared in methylcellulose (Table 27).

This example demonstrates that Compound 1 2HCl and Compound 2 free base may be administered at higher oral doses to achieve a similar bioavailability compared to the maximum tolerated intravenous dose of each compound.

TABLE 24
Comparison of group mean pharmacokinetic parameters calculated for Compound 2
among the different doses and formulations administered to Sprague Dawley rats.

| | Dose (mg/kg) | Tmax (hr) | Cmax (ng/mL) | AUC₀₋ₗₐₛₜ (hr*ng/mL) |
|---|---|---|---|---|
| Group | Route - Vehicle | Mean ± StdDev | Mean ± StdDev | Mean ± StdDev |
| 1 | 24 | 0.90 ± 0.22 | 320.52 ± 111.14 | 975.25 ± 304.03 |
| | PO | | | |
| | ORA-Plus | | | |
| 5 | 24 | 1.80 ± 0.45 | 684.96 ± 109.43 | 2013.57 ± 175.74 |
| | PO | | | |
| | 60 mM Citric acid buffer + | | | |
| | 30% Captisol ® | | | |
| 2 | 36 | 1.50 ± 1.41 | 747.37 ± 237.98 | 2683.67 ± 810.69 |
| | PO | | | |
| | ORA-Plus | | | |
| 6 | 36 | 2.40 ± 2.07 | 830.87 ± 618.10 | 2943.34 ± 1571.78 |
| | PO | | | |
| | 60 mM Citric acid buffer + | | | |
| | 30% Captisol ® | | | |
| 3 | 48 | 2.70 ± 1.79 | 1243.87 ± 519.08 | 5217.04 ± 2764.37 |
| | PO | | | |
| | ORA-Plus | | | |
| 7 | 48 | 1.40 ± 0.55 | 1396.89 ± 626.48 | 5506.00 ± 2592.20 |
| | PO | | | |
| | 60 mM Citric acid buffer + | | | |
| | 30% Captisol ® | | | |
| 4 | 60 | 3.00 ± 2.74 | 909.29 ± 302.21 | 3555.64 ± 905.93 |
| | PO | | | |
| | ORA-Plus | | | |
| 8 | 60 | 4.40 ± 1.67 | 1082.51 ± 583.74 | 4745.09 ± 3072.21 |
| | PO | | | |
| | 60 mM Citric acid buffer + | | | |
| | 30% Captisol ® | | | |
| 9 | 12 | 0.25 ± 0.00 | 2355.16 ± 92.71 | 3390.71 ± 402.22 |
| | IV | | | |
| | 5 mM Citric acid buffer + | | | |
| | 15% Captisol ® | | | |
| 10 | 24 | 0.25 ± 0.00 | 5109.40 ± 415.58 | 7497.50 ± 551.76 |
| | IV | | | |
| | 5 mM Citric acid buffer + | | | |
| | 15% Captisol ® | | | |

TABLE 25
Comparison of group mean pharmacokinetic parameters calculated for Compound 1
among the different doses and formulations administered to Sprague Dawley rats.

| | Dose (mg/kg) | Tmax (hr) | Cmax (ng/mL) | AUC₀₋ₗₐₛₜ (hr*ng/mL) |
|---|---|---|---|---|
| Group | Route - | Mean ± StdDev | Mean ± StdDev | Mean ± StdDev |
| 11 | 24 | 2.00 ± 0.00 | 807.41 ± 213.51 | 2704.22 ± 461.53 |
| | PO | | | |
| | ORA-Plus | | | |
| 12 | 36 | 2.00 ± 0.00 | 853.02 ± 193.37 | 3215.68 ± 870.00 |
| | PO | | | |
| | ORA-Plus | | | |
| 15 | 36 | 2.20 ± 1.10 | 811.74 ± 269.81 | 2854.03 ± 919.15 |
| | PO | | | |
| | 0.5% | | | |
| | Methylcellulose | | | |
| | in water | | | |
| 13 | 48 | 2.40 ± 0.89 | 1420.03 ± 469.82 | 6502.71 ± 2027.82 |
| | PO | | | |
| | ORA-Plus | | | |
| 16 | 48 | 1.40 ± 0.55 | 1645.26 ± 270.63 | 6503.64 ± 1688.97 |
| | PO | | | |
| | 0.5% | | | |
| | Methylcellulose | | | |
| | in water | | | |
| 14 | 60 | 3.00 ± 2.00 | 1119.69 ± 174.94 | 4866.92 ± 1415.66 |
| | PO | | | |
| | ORA-Plus | | | |
| 17 | 60 | 1.50 ± 0.71 | 1761.92 ± 457.97 | 7322.91 ± 2442.50 |
| | PO | | | |
| | 0.5% | | | |
| | Methylcellulose | | | |
| | in water | | | |
| 18 | 12 | 0.25 ± 0.00 | 1277.23 ± 325.03 | 2466.18 ± 572.93 |
| | IV | | | |
| | 0.9% Saline | | | |
| 19 | 24 | 0.31 ± 0.13 | 2080.52 ± 79.32 | 5503.84 ± 2800.58 |
| | IV | | | |
| | 0.9% Saline | | | |

TABLE 26
Comparison of Cₘₐₓ and AUC₀₋ₗₐₛₜ of oral solutions prepared
in ORA-plus ® relative to those prepared in citrate
buffer- Captisol ®combination for Compound 2 from
the different doses to Sprague Dawley rats. Calculations
were based on values from the animals in ORA-plus ® groups
relative to the values from animals receiving citrate buffer-
Captisol ®groups.

| | | | % Cₘₐₓ | % AUC₀₋ₗₐₛₜ |
|---|---|---|---|---|
| Group # for | Group # for | Dose | (ng/mL) | (hr*ng/mL) |
| test | reference | (mg/ | ORA vs Citrate | ORA vs Citrate |
| formulation | formulation | kg) | Mean ± StdDev | Mean ± StdDev |
| 1 | 5 | 24 | 47.65 ± 16.52 | 48.63 ± 15.03 |
| 2 | 6 | 36 | 89.97 +/− 82.63 | 91.18 +/− 72.46 |
| 3 | 7 | 48 | 99.04 +/− 80.01 | 94.75 +/− 64.83 |
| 4 | 8 | 60 | 83.99 +/− 73.13 | 75.93 +/− 63.12 |

TABLE 27
Comparison of % of Cₘₐₓ and AUC₀₋ₗₐₛₜ of oral solutions
prepared in ORA-plus ® relative to methylcellulose
for Compound 1 from the different doses to Sprague Dawley
rats. Calculations were based on values from the animals
in ORA-plus ® groups relative to the values from
animals receiving methylcellulose groups.

| | | | % Cₘₐₓ | % AUC₀₋ₗₐₛₜ |
|---|---|---|---|---|
| | | | (ng/mL) | (hr*ng/mL) |
| Group # for | Group # for | Dose | ORA vs | ORA vs |
| test | reference | (mg/ | methylcellulose | methylcellulose |
| formulation | formulation | kg) | Mean ± StdDev | Mean ± StdDev |
| 12 | 15 | 36 | 118.75 ± 64.56 | 119.11 ± 45.91 |
| 13 | 16 | 48 | 89.30 ± 37.98 | 111.81 ± 69.18 |
| 14 | 17 | 60 | 66.16 ± 17.44 | 70.33 ± 23.05 |

TABLE 28
Comparison of % Cₘₐₓ and AUC₀₋ₗₐₛₜ of oral (PO) solutions
prepared in ORA-plus ® and methylcellulose for
Compound 1 relative to the intravenous dose (IV) at 24
mg/kg (0.9% saline) administered to Sprague Dawley rats.
Calculations were based on values from the animals in PO
groups relative to the values from animals in IV groups.

| | | | % Cₘₐₓ | % AUC₀₋ₗₐₛₜ |
|---|---|---|---|---|
| | | | (ng/mL) | (hr*ng/mL) |
| Group # for | Group # for | Dose | Oral vs IV | Oral vs IV |
| test | reference | (mg/ | (24 mg/kg) | (24 mg/kg) |
| formulation | formulation | kg) | Mean ± StdDev | Mean ± StdDev |
| 11 | Group 19 - | 24 | 38.81 ± 10.26 | 49 ± 8.39 |
| 12 | IV | 36 | 41.00 ± 9.29 | 54.83 ± 15.81 |
| 13 | 24 mg/kg | 48 | 68.25 ± 22.58 | 118.15 ± 36.84 |
| 14 | 0.9% Saline | 60 | 53.82 ± 8.41 | 88.43 ± 25.72 |
| 15 | | 36 | 39.02 ± 12.97 | 137.18 ± 44.18 |
| 16 | | 48 | 79.08 ± 13.01 | 118.16 ± 8.18 |
| 17 | | 60 | 84.69 ± 22.01 | 133.03 ± 17.4 |

### Example 6

This Example examined and compared the pharmacokinetic (PK) parameters after a single administration in rats of Compound 2 free base and Compound 2 2HCl prepared in ORA-Plus® or SyrSpend® drinking solution. Similarly, PK parameters of Compound 1 2HCl prepared in ORA-Plus® solution was compared to SyrSpend® SF Cherry solution.

### Materials and Methods

The animals used in this study were female Sprague Dawley Rats physiologically normal with Jugular vein cannulas (JVC) supplied by Envigo. At the time of receipt, mice were 200-224 g in weight. Seventy total animals were used and animals were not replaced during the course of the study. The animals were identified by indelible markings. The animals were housed in individually ventilated microisolator cages and allowed to acclimate 11-12 days post-surgery and 7-8 days in-house. The animals were maintained under pathogen-free conditions and given Teklad Global Diet® 2920x irradiated pellets for food and autoclaved water ad libitum.

Compound 2 provided in free base form was stored at −20° C., protected from light. For oral administration of Compound 2 free base in ORA-Plus® drinking solution, Compound 2 free base was suspended in drinking solution ORA-Plus® (Perrigo; Minneapolis, Minn.). First, a mortar and pestle was used to smooth out the Compound 2 free base powder, then a small amount of ORA-Plus® was added, and next, the mixture was triturated to a thick, smooth paste. The remainder of the ORA-Plus® was added by geometric dilution. The Compound 2 free base and ORA-Plus® mixture was dispensed in a tight, light resistant amber bottle with appropriate labeling. This mixture was shaken well before using, protected from light and this formulation appeared to be in suspension. For oral administration of Compound 2 free base in SyrSpend® SF Cherry solution (Fagron Inc.; St. Paul, Minn.), a mortar and pestle was used to smooth out the Compound 2 free base powder and a small amount of SyrSpend® SF was added and the mixture was triturated to a thick, smooth paste. The remainder of the SyrSpend® SF was added by geometric dilution. The SyrSpend® and Compound 2 free base mixture was dispensed in a tight, light resistant amber bottle with appropriate labeling. This mixture was shaken well before use and protected from light. This formulation appeared to be a suspension. Compound 2 free base in SyrSpend® SF Cherry solution and in ORA-Plus® solution were made fresh immediately prior to use.

Compound 2 provided in 2HCl form was stored at −20° C., protected from light. For oral administration of Compound 2 HCl in ORA-Plus® drinking solution, a mortar and pestle was used to smooth out the Compound 2 2HCl powder and a small amount of ORA-Plus® was added and the mixture was triturated to a thick, smooth paste. The remainder of the ORA-Plus® was added by geometric dilution. The Compound 2 HCl and ORA-Plus® mixture was dispensed in a tight, light resistant amber bottle with appropriate labeling. This mixture was shaken well before using and protected from light. This formulation appeared to be a suspension. For oral administration of Compound 2 HCl in SyrSpend® SF Cherry solution, a mortar and pestle was used to smooth out the Compound 2 2HCl powder and a small amount of SyrSpend® SF was added and the mixture was triturated to a thick, smooth paste. The remainder of the SyrSpend® SF was added by geometric dilution. The mixture of Compound 2 2HCl in SyrSpend® SF Cherry was dispensed in a tight, light resistant amber bottle with appropriate labeling. This mixture was shaken well before using and protected from light.

The salt:base ratio is 1.14:1 (a correction factor of 1.14 was applied to the Compound 2 dihydrochloride salt to obtain the correct amount of Compound 2 free base). Dose levels of Compound 2 were based on the free base, not the salt. Solubility at ˜20-25 mg/ml was achieved for the 2HCl salt at pH˜2.5. pH will drop as 2HCl is added into the SyrSpend® SF Solution. Dosage forms of Compound 2 2HCl in ORA-Plus® and in SyrSpend® SF Cherry appeared to be suspension instead of clear solutions. Final physical appearance matched that of the vehicle used. Due to opaque properties of vehicles, full solubility could not be confirmed. However, resultant dosing material appeared homogenous. Dosage forms of Compound 2 2HCl in ORA-Plus® and in SyrSpend® SF Cherry were made fresh immediately prior to use.

Compound 1 dihydrochloride (2HCl) was provided as a crystalline powder and stored at 4° C. protected from light. The administered form of Compound 1 2HCl was a suspension. Dosage form of Compound 1 2HCl appeared to be a suspension instead of a clear solution as indicated in the protocol. Final physical appearance matched that of the vehicle used. Due to opaque properties of vehicles, full solubility could not be confirmed. However, resultant dosing material appeared homogenous. For oral administration of Compound 1 2HCl suspended in ORA-Plus® drinking solution, a mortar and pestle was used to smooth out the powder and a small amount of ORA-Plus® was added and the mixture was triturated to a think, smooth paste. The remainder of the ORA-Plus® was added by geometric dilution. The Compound 1 2HCl and ORA-Plus® mixture was dispensed in a tight, light resistant amber bottle with appropriate labeling. This mixture was shaken well before using, protected from light. This formulation appeared to be a suspension. For oral administration of Compound 1 2HCl in SySpend® SF Cherry, a mortar and pestle was used to smooth out the Compound 1 2HCl powder. A small amount of SyrSpend® SF was added and the mixture was triturated to a thick, smooth paste. The reaminder of the SyrSpend® SF was added by geometric dilution. The mixture of Compound 1 2HCl and SyrSpend® SF was dispensed in a tight, light resistant amber bottle with appropriate labeling. This mixture was shaken well before using and protected from light. This formulation appeared to be a suspension.

Dosage forms of Compound 1 2HCl in ORA-Plus® and in SyrSpend® SF Cherry appeared to be suspensions instead of clear solutions. Final physical appearance matched that of the vehicle used. Due to opaque properties of vehicles, full solubility could not be confirmed. However, resultant dosing material appeared homogenous. The salt:base ratio is 1.14:1 (A correction factor of 1.14 was applied to the Compound 1 dihydrochloride salt to obtain the correct amount of Compound 1 free base). Dose levels of Compound 1 were based on the free base, not the salt. Dosage forms of Compound 1 2HCl in ORA-Plus® solution and in SyrSpend® SF solution were made fresh immediately prior to use.

500 μl of each dosing mixture at each concentration was retained at time of preparation for concentration confirmation. Each dosing mixture was stored at 4° C. for 5-10 minutes prior to analysis.

Animals were randomized using random equilibration of body weights on Day 1 into one of 14 study groups, as shown in Table 29 (Groups 1-14), with 5 animals in each group, Body weights were collected Days 1, 2, 3, and/or 4 to accommodate data collection of staggered groups. Gross observations were noted during the course of the study. Treatment initiation was staggered by group to accommodate collections, resulting in multiple treatment initiation days. Therefore, treatment was initated on Day 1, 2, 3 or 4. The study endpoint followed the final collected timepoint for each group.

TABLE 29
Study Groupings.

| | | | Compound 2 | Compound 2 | Compound 1 | Compound 2 | Compound 2 | Compound 1 |
|---|---|---|---|---|---|---|---|---|
| | | | Free Base | 2HCl | 2HCl | Free Base | 2HCl | 2HCl |
| | | | (ORAPlus ®) | (ORAPlus ®) | (ORAPlus ®) | (SyrSpend ® SF) | (SyrSpend ® SF) | (SyrSpend ® SF) |
| Group | | N | [Single Dose] | [Single Dose] | [Single Dose] | [Single Dose] | [Single Dose] | [Single Dose] |
| 1. | Compound 2 | 5 | X | | | | | |
| | Free Base in | | | | | | | |
| | ORA-Plus ® 24 | | | | | | | |
| | mg/kg (PO) | | | | | | | |
| 2. | Compound 2 | 5 | X | | | | | |
| | Free Base in | | | | | | | |
| | ORA-Plus ® 48 | | | | | | | |
| | mg/kg (PO) | | | | | | | |
| 3. | Compound 2 | 5 | | X | | | | |
| | 2HCl in ORA- | | | | | | | |
| | Plus ® 24 | | | | | | | |
| | mg/kg (PO) | | | | | | | |
| 4. | Compound 2 | 5 | | X | | | | |
| | 2HCl in ORA- | | | | | | | |
| | Plus ® 48 | | | | | | | |
| 5. | Compound 1 | 5 | | | X | | | |
| | 2HCl in ORA- | | | | | | | |
| | Plus ® 24 | | | | | | | |
| | mg/kg (PO) | | | | | | | |
| 6. | Compound 1 | 5 | | | X | | | |
| | 2HCl in ORA- | | | | | | | |
| | Plus ® 48 | | | | | | | |
| | mg/kg (PO) | | | | | | | |
| 7. | Compound 2 | 5 | | | | X | | |
| | Free Base in | | | | | | | |
| | SyrSpend ® SF | | | | | | | |
| | 24 mg/kg (PO) | | | | | | | |
| 8. | Compound 2 | 5 | | | | X | | |
| | Free Base in | | | | | | | |
| | SyrSpend ® SF | | | | | | | |
| | 48 mg/kg (PO) | | | | | | | |
| 9. | Compound 2 | 5 | | | | | X | |
| | 2HCl in | | | | | | | |
| | SyrSpend ® SF | | | | | | | |
| | 24 mg/kg (PO) | | | | | | | |
| 10. | Compound 2 | 5 | | | | | X | |
| | 2HCl in | | | | | | | |
| | SyrSpend ® SF | | | | | | | |
| | 48 mg/kg (PO) | | | | | | | |
| 11. | Compound 2 | 5 | | | | | X | |
| | 2HCl | | | | | | | |
| | SyrSpend ® SF | | | | | | | |
| | 60 mg/kg (PO) | | | | | | | |
| 12. | Compound 1 | 5 | | | | | | X |
| | 2HCl in | | | | | | | |
| | SyrSpend ® SF | | | | | | | |
| | 24 mg/kg (PO) | | | | | | | |
| 13. | Compound 1 | 5 | | | | | | X |
| | 2HCl in | | | | | | | |
| | SyrSpend ® SF | | | | | | | |
| | 48 mg/kg (PO) | | | | | | | |
| 14. | Compound 1 | 5 | | | | | | X |
| | 2HCl in | | | | | | | |
| | SyrSpend ® SF | | | | | | | |
| | 60 mg/kg (PO) | | | | | | | |

Groups 1-2 received a single dose of Compound 2 Free base in ORA-Plus® solution at an administered volume of 10 mL/kg via oral gavage at the dose indicated in Table 29.

Groups 3-4 received a single dose of Compound 2 2HCl in ORA-Plus® solution at an administered volume of 10 mL/kg via oral gavage at the dose indicated in Table 29.

Groups 5-6 received a single dose of Compound 1 2HCl in ORA-Plus® solution at an administered volume of 10 mL/kg via oral gavage at the dose indicated in Table 29.

Groups 7-8 received a single dose of Compound 2 Free Base in SyrSpend® SF solution at an administered volume of O1 mL/kg via oral gavage at the dose indicated in Table 29.

Groups 9-11 received a single dose of Compound 2 2HCl in SyrSpend® SF solution at an administered volume of O1 mL/kg via oral gavage at the dose indicated in Table 29.

Groups 12-14 received a single dose of Compound 1 2HCl in SyrSpend® SF solution at an administered volume of O1 mL/kg via oral gavage at the dose indicated in Table 29.

Whole Blood was collected from all rats in all groups via jugular vein cannulas pre-dose (T=0), and at 0.5, 1,2, 4, 6, 8, and 24 hours post dose. Blood was placed in li-heparin microtainers (Becton, Dickinson & Co; Franklin Lakes, N.J.), centrifuged at 4° C., and processed for plasma. Plasma was removed and placed into a cryovial (Thermo Scientific; Rochester, N.Y.), snap frozen in liquid nitrogen, and stored at −80° C. A sufficient amount of blood was collected from all rats to yield enough plasma for PK analysis.

### Pharmacokinetic Analysis

Samples were analyzed for levels of Compound 2 Free Base, Compound 2 2HCl and Compound 1 2HCl by LC-MS/MS.

### Standards

Provided Compound 2 free base, Compound 2 2HCl and Compound 1 2HCl and Compound 2 d4 (internal standard) was weighed out for preparation of stocks solutions in DMSO. These solutions were used to spike into plasma for preparation of appropriate standard curves.

### Data Collection

MassLynx software (Waters corp.): Raw data generated.

### Methods: LCMS Analysis and Pharmacokinetic Analysis

For Compound 2 samples, methods were used described in Example 5, except minor adjustments were made to provided bioanalytical methods as needed.

### Bioanalytical Methods-Compound 2 & Compound 1

Plasma samples were processed for extraction of compounds using protein precipitation and centrifugation. Supernatant from samples were then analyzed against standard calibrators similarly prepared in blank plasma, using a Xevo-TQS mass spectrometer coupled to Acquity UPLC system. Separation was conducted using the appropriate analytical column with analytes monitored in MRM mode. Calibration curve was used to calculate concentration of quality control samples by interpolation and accuracy assessed.

### Pharmacokinetic Analysis

Calculated concentrations per time points were used for noncompartmental pharmacokinetic analysis using Phoenix WinNonLin software (v. 6.4). Parameters such as maximal concentration achieved (Cₘₐₓ), time to Cₘₐₓ (Tₘₐₓ), area under the curve (AUC), half-life (t½), volume of distribution and clearance were reported. For some animals, no clear terminal phase was available, therefore extrapolated values were not included and noted when relevant.

Plasma PK parameters for individual animals in all groups were calculated. PK parameters were labeled as N/A to indicate that one or more of the selection criteria (outlined in Table 35) were not met by the plasma distribution of the individual animal to allow accurate calculations of the value. Samples collected previous to compound dosing and labeled as “0” had no plasma Compound 2 levels and were reported as below limit of quantitation (BLQ).

### Results

Compound 2 free base in ORA-Plus® or in SyrSpend® showed similar PK values for the respective doses tested. Summaries of PK parameters calculated for Compound 2 free base and 2HCl in ORA-Plus® or SyrSpend® are shown in Tables 30 to 32. Likewise, Compound 2 2HCl PK parameters are also comparable for each preparation. Results also showed that, overall, PK parameters between Compound 2 free base and Compound 2 2HCl in either drinking solution were comparable (Table 36).

All animals had quantifiable plasma levels of Compound 2 up to the 8-hour time point and some animals showed levels remaining at 24-hour time point as presented in the tables.

Table 36 is a comparison of AUC_{0_last} for Compound 2 free base or 2HCl salt prepared in ORA-Plus® or SyrSpend® at different doses. Calculations were based on the ratio of the values from average calculations obtained in the test formulation groups relative to the average values from reference groups as indicated. In brief, AUC₀₋ₗₐₛₜ for Compound 2 free base at 24 mg/kg in ORA-Plus® (Group 1) is 123.40% of that in SyrSpend® (Group 7) and 121.69% of Compound 2 2HCl (Group 3). AUC_{0_last} for COMPOUND 2 2HCl at similar dose in ORA-Plus® (Group 3) is 109.55% of that in SyrSpend® (Group 9). AUC₀₋ₗₐₛₜ for Compound 2 free base in SyrSpend® (Group 8) is 94.91% of COMPOUND 2 2HCL in SyrSpend® (Group 10). Compound 2 2HCl exposure expressed as AUC_{0_last} for the SyrSpend® dosed groups at 24, 48 and 60 mg/kg (Group 9, 10 and 11), showed increase in overall exposure although less than linear (r2=0.43, data not shown).

The second part of this study was to compare PK parameters in ORA-Plus® and SyrSpend® solution for Compound 1 2HCl. The results indicate that the exposure from these two formulations are similar. All animals had quantifiable plasma levels of Compound 1 2HCl up to the 8-hour time point and some animals showed remaining plasma levels up to the 24-hour time points (data not shown). Tables 33 to 34, shows the summary data of the PK parameters for groups 5 and 6, and 12 to 14 receiving Compound 1 2HCl, prepared in ORA-Plus® or SyrSpend®.

Table 37 is a comparison of AUC_{0_last} for Compound 1 2HCl prepared in ORA-Plus® or SyrSpend® solutions at all concentrations tested. Calculations were based on the ratio of the values from average calculations of AUC₀₋ₗₐₛₜ obtained in the test formulation groups relative to the average values from reference groups as indicated. AUC_{0_last} for the 24 mg/kg dose group in ORA-Plus® (Group 5) is 84.12% of SyrSpend® (Group 12), while the AUC₀₋ₗₐₛₜ for the 48 mg/kg dose group in ORA-Plus® (Group 6) is 298.14% of that in SyrSpend® (Group 13). However, examination of the exposure expressed as AUC_{0_last} for the SyrSpend® dosed groups (Group 12, 13 and 14) shows increase in overall exposure for COMPOUND 1 with dose for the groups receiving 24 and 60 mg/kg, although the increase is less than linear (r2=0.35, data not shown) when considering the group receiving 48 mg/kg. Indeed, a comparison of the AUC_{0_last} of the 48 mg/kg group in ORA-Plus® to the 60 mg/kg group in SyrSpend®, after correcting for the 1.25 increase in dose, indicates that the exposure from these two preparations are similar.

All groups exhibited weight gain or minimal group body weight loss that was not impactful to the study (data not shown). No negative clinical observations were recorded throughout the study. The lack of clinical observations combined with no appreciable body weight loss indicates that the doses were well-tolerated within the short timeframe of this study.

This Example showed that both Compound 1 (2HCl) and Compound 2 (free base or 2HCl), when prepared in either drinking solution, are able to achieve comparable exposure with minimal toxicity, while administered orally to rats.

TABLE 30
Summary of pharmacokinetic parameters calculated for Compound
2 (free base or 2HCl) from plasma analysis following single oral
dose of 24 or 48 mg/kg administered to Sprague Dawley rats.

| | Group Dose (mg/kg) Vehicle | | |
|---|---|---|---|
| | G1 | G2 | G3 |
| | Free base | Free base | 2HCl |
| | 24 mg/kg | 48 mg/kg | 24 mg/kg |
| Parameter Name | ORA-plus | ORA-plus | ORA-plus |
| Half-life (hr) | 2.36 ± 1.98 | 2.61 ± 0.44* | 2.34 ± 1.76 |
| Tmax (hr) | 1.40 ± 0.55 | 2.80 ± 1.64 | 1.60 ± 0.55 |
| Cmax (ng/mL) | 578.93 ± 107.89 | 803.30 ± 278.16 | 407.22 ± 277.53 |
| AUC0-last (hr*ng/mL) | 1568.22 ± 152.42 | 4456.29 ± 2109.02 | 1288.69 ± 665.76 |
| AUC0-∞ (hr*ng/mL) | 1612.81 ± 155.88 | 5060.82 ± 2069.08* | 1336.87 ± 678.44 |
| AUC % Extrap | 2.77 ± 0.30 | 6.22 ± 6.79* | 4.00 ± 1.84 |
| Vz_F (L/kg) | 50.18 ± 39.85 | 38.84 ± 10.64* | 81.00 ± 80.22 |
| Cl_F (L/hr/kg) | 14.99 ± 1.45 | 10.63 ± 3.93* | 21.44 ± 9.28 |

*n = 4

TABLE 31
Summary of pharmacokinetic parameters calculated for Compound
2 (free base or 2HCl) from plasma analysis following single oral
dose of 24 or 48 mg/kg administered to Sprague Dawley rats.

| | Group Dose (mg/kg) Vehicle | | |
|---|---|---|---|
| | G4 | G7 | G8 |
| | 2HCl | Free base | Free base |
| | 48 mg/kg | 24 mg/kg | 48 mg/kg |
| Parameter name | ORA-plus | SyrSpend | SyrSpend |
| Half-life (hr) | 2.18 ± 0.35 | 2.78 ± 1.13* | 3.56 ± 1.03 |
| Tmax (hr) | 2.20 ± 1.10 | 1.00 ± 0.00 | 3.40 ± 1.95 |
| Cmax (ng/mL) | 1120.02 ± 428.84 | 370.51 ± 195.86 | 1034.02 ± 420.84 |
| AUC₀₋ₗₐₛₜ (hr*ng/mL) | 6324.80 ± 3214.15 | 1270.85 ± 523.90 | 8144.51 ± 3551.90 |
| AUC_{0-∞} (hr*ng/mL) | 6369.07 ± 3168.14 | 1177.62 ± 450.81* | 8081.58 ± 4089.12 |
| AUC % Extrap | 1.32 ± 2.64 | 6.89 ± 3.49* | 1.71 ± 1.37 |
| Vz_F (L/kg) | 28.42 ± 13.10 | 88.96 ± 44.19* | 44.20 ± 37.62 |
| Cl_F (L/hr/kg) | 9.25 ± 4.59 | 22.18 ± 6.32* | 8.18 ± 6.30 |

*n = 4

TABLE 32
Summary of pharmacokinetic parameters calculated for Compound 2
(free base or 2HCl) from plasma analysis following single oral
dose of 24, 48 or 60 mg/kg administered to Sprague Dawley rats.

| | Group Dose (mg/kg) Vehicle | | |
|---|---|---|---|
| | G9 | G10 | G11 |
| | 2HCl | 2HCl | 2HCl |
| | 24 mg/kg | 48 mg/kg | 60 mg/kg |
| Parameter name | SyrSpend | SyrSpend | SyrSpend |
| Half-life (hr) | 3.41 ± 1.24 | 4.28 ± 0.04** | 2.84 ± 0.83* |
| Tmax (hr) | 1.20 ± 0.45 | 4.00 ± 1.41 | 2.60 ± 1.34 |
| Cmax (ng/mL) | 269.84 ± 184.40 | 1137.73 ± 310.53 | 824.82 ± 246.53 |
| AUC₀₋ₗₐₛₜ (hr*ng/mL) | 1176.34 ± 688.15 | 8580.90 ± 2221.06 | 4890.68 ± 1309.78 |
| AUC_{0-∞} (hr*ng/mL) | 1236.21 ± 716.55 | 6564.19 ± 1221.72** | 4948.57 ± 1779.21* |
| AUC % Extrap | 4.72 ± 4.49 | 2.05 ± 0.35** | 0.68 ± 0.89* |
| Vz_F (L/kg) | 129.96 ± 81.45 | 45.95 ± 8.11** | 55.47 ± 25.73* |
| Cl_F (L/hr/kg) | 24.35 ± 12.26 | 7.44 ± 1.38** | 13.15 ± 4.35* |

**n = 2;
*n = 4

TABLE 33
Summary of pharmacokinetic parameters calculated for Compound
1 (2HCl) from plasma analysis following single oral dose
of 24 or 48 mg/kg administered to Sprague Dawley rats.

| | Group Dose (mg/kg) Vehicle | | |
|---|---|---|---|
| | G5 | G6 | G12 |
| | 2HCl | 2HCl | 2HCl |
| | 24 mg/kg | 48 mg/kg | 24 mg/kg |
| Parameter Name | ORA-plus | ORA-plus | SyrSpend |
| Half-life (hr) | 1.80 ± 0.47* | 3.50 ± 0.53 | 2.91^{a} |
| Tmax (hr) | 2.40 ± 0.89 | 2.80 ± 1.10 | 3.20 ± 1.10 |
| Cmax (ng/mL) | 1065.48 ± 221.20 | 1117.11 ± 428.61 | 1031.28 ± 151.22 |
| AUC₀₋ₗₐₛₜ (hr*ng/mL) | 4203.02 ± 1115.77 | 7928.74 ± 2380.84 | 4996.27 ± 1263.26 |
| AUC_{0-∞} (hr*ng/mL) | 4252.91 ± 1227.57* | 8040.09 ± 2409.87 | 5754.70^{a} |
| AUC % Extrap | 5.00 ± 4.92* | 1.42 ± 0.79 | 0.47^{a} |
| Vz_F (L/kg) | 15.12 ± 3.53* | 33.74 ± 16.37 | 17.51^{a} |
| Cl_F (L/hr/kg) | 6.14 ± 2.32* | 6.63 ± 2.82 | 4.17^{a} |

*n = 4;
^{a}n = 1;

TABLE 34
Summary of pharmacokinetic parameters calculated for Compound
1 (2HCl) from plasma analysis following single oral dose of
24, 48 or 60 mg/kg administered to Sprague Dawley rats.

| | Group Dose (mg/kg) Vehicle | |
|---|---|---|
| | G13 | G14 |
| | 2HCl | 2HCl |
| | 48 mg/kg | 60 mg/kg |
| Parameter Name | SyrSpend | SyrSpend |
| Half-life (hr) | 4.42 ± 1.52** | 3.20 ± 0.40*** |
| Tmax (hr) | 0.70 ± 0.27 | 2.40 ± 2.07 |
| Cmax (ng/mL) | 1989.85 ± 786.96 | 1705.33 ± 314.17 |
| AUC₀₋ₗₐₛₜ (hr*ng/mL) | 2659.41 ± 945.87 | 12626.51 ± 5096.26 |
| AUC_{0-∞} (hr*ng/mL) | 2170.01 ± 547.52** | 11494.10 ± 4436.27*** |
| AUC % Extrap | 6.76 ± 5.82** | 0.69 ± 0.34*** |
| Vz_F (L/kg) | 139.42 ± 13.22** | 27.15 ± 11.86*** |
| Cl_F (L/hr/kg) | 22.85 ± 5.76** | 5 72 ± 1.99*** |

**n = 2;
***n = 3

TABLE 35
Summary table of pharmacokinetic parameters used,
its definition and criteria for data analysis.

| PK parameters | Criteria | |
|---|---|---|
| Rsq-adjusted | | ≥0.85 |
| (R²) | | |
| Data Points | | 3 or more |
| Tmax (hr) | 1. | Cannot be included in the regression |
| | 2. | Optimal between 1-3 hr |
| C₀ | | In the case of IV dosing, C₀ must be greater than |
| | | Cₘₐₓ |
| Half-life (hr) | 1. | The time required for the concentration to fall to |
| | | 50% of its initial value |
| | 2. | ≥half the last time point for which data is available |
| AUC_{0-∞} | | Must be greater than AUC₀₋ₗₐₛₜ |
| AUC % Extrap | | 25-30% or less |
| Vd (Vss or | | >10 L/kg = High; <1 L/kg = Low |
| Vz/F) | | |
| Cl | | >4.0 L/hr/kg = High; <1.2 L/hr/kg = Low |
| % F | | >50% = high; <20% = low |

TABLE 36
Comparison of AUC₀₋ₗₐₛₜ of oral solutions prepared in ORA-plus ®
or SyrSpend ® for Compound 2, free base or 2HCl salt, from the different
doses to Sprague Dawley rats. Calculations were based on the ratio of the
values from average calculations obtained in the test formulation groups
relative to the average values from reference groups as indicated.

| Group # for | Group # for | Dose - Vehicle for | Dose - Vehicle for | % AUC0-last |
|---|---|---|---|---|
| test | reference | test formulation | reference formulation | (hr*ng/mL) |
| formulation | formulation | (mg/kg) | (mg/kg) | test vs reference |
| 1 | 3 | 24 - FB ORA-plus | 24 - 2HCl ORA-plus | 121.69 |
| 1 | 4 | 24 - FB ORA-plus | 48 - 2HCl ORA-plus | 24.79 |
| 1 | 7 | 24 - FB ORA-plus | 24 - FB - SyrSpend | 123.40 |
| 1 | 9 | 24 - FB ORA-plus | 24 - 2HCl SyrSpend | 133.31 |
| 2 | 4 | 48 - FB ORA-plus | 48 - 2HCl ORA-plus | 70.46 |
| 2 | 8 | 48 - FB ORA-plus | 48 - FB SyrSpend | 54.72 |
| 2 | 10 | 48 - FB ORA-plus | 48 - 2HCl SyrSpend | 51.93 |
| 3 | 9 | 24 - 2HCl ORA-plus | 24 - 2HCl SyrSpend | 109.55 |
| 4 | 10 | 48 - 2HCl ORA-plus | 48 - 2HCl SyrSpend | 73.71 |
| 7 | 9 | 24 -FB SyrSpend | 24 - 2HCl SyrSpend | 108.03 |
| 8 | 10 | 48 - FB SyrSpend | 48 - 2HCl SyrSpend | 94.91 |
| 9 | 10 | 24 - 2HCl SyrSpend | 48 - 2HCl SyrSpend | 13.71 |
| 9 | 11 | 24 - 2HCl SyrSpend | 60 - 2HCl SyrSpend | 24.05 |
| 10 | 11 | 48 - 2HCl SyrSpend | 60 - 2HCl SyrSpend | 175.45 |

FB = free base
2HCl = salt form

TABLE 37
Comparison of AUC₀₋ₗₐₛₜ of oral solutions prepared in ORA-plus ® or SyrSpend ® for
Compound 1 2HCl salt, and dosed at 24, 48 or 60 mg/kg to Sprague Dawley rats. Calculations
were based on the ratio of the values from average calculations obtained in the test formulation
groups relative to the average values from reference groups as indicated.

| Group # for | Group # for | Dose - Vehicle for | Dose - Vehicle for | % AUC₀₋ₗₐₛₜ |
|---|---|---|---|---|
| test | reference | test formulation | reference formulation | (hr*ng/mL) |
| formulation | formulation | (mg/kg) | (mg/kg) | test vs reference |
| 5 | 6 | 24 - 2HCl ORA-plus | 48 - 2HCl ORA-plus | 53.01 |
| 5 | 12 | 24 - 2HCl ORA-plus | 24 - 2HCl SyrSpend | 84.12 |
| 6 | 13 | 48 - 2HCl ORA-plus | 48 - 2HCl SyrSpend | 298.14 |
| 6 | 14 | 48 - 2HCl ORA-plus | 60 - 2HCl SyrSpend | 62.79 |
| 12 | 13 | 24 - 2HCl SyrSpend | 48 - 2HCl SyrSpend | 187.87 |
| 12 | 14 | 24 - 2HCl SyrSpend | 60 - 2HCl SyrSpend | 39.57 |
| 13 | 14 | 48 - 2HCl SyrSpend | 60 - 2HCl SyrSpend | 21.06 |

2HCL = salt form

### Example 7

This Example examined drinking solution vehicles for Compound 1 2HCl. Initially Orasweet® Sugar Free options were explored as a vehicle for Compound 1 2HCl.

### Materials and Methods

ORA-Sweet®, commerically available from Perrigo, comprises purified water, sucrose, glycerine, sorbitol, and flavouring. ORA-Sweet® is buffered with citric acid and sodium phosphate and preserved with methylparaben and potassium sorbate.

ORA-Sweet® Sugar Free, commerically available from Perrigo, comprises purified water, glycerine, sorbitol, sodium saccharin, xanthan gum, and flavouring. It is buffered with citric acid and sodium citrate and preserved with methylparaben (0.03%), potassium sorbate (0.1%), and propylparaben (0.008%).

SyrSpend® SF Cherry, commercially available from Fargon, comprises purified water, modified food starch, sodium citrate, citric acid, sucralose, sodium benzoate (<0.1% preservative), sorbic acid, malic acid and simethicone.

SyrSpend® SF Alka, commercially available from Fargon, comprises modified starch, calcium carbonate and sucralose.

ORA-Blend®, commerically available from Perrigo, comprises purified water, sucrose, glycerin, sorbitol, flavoring, microcrystalline cellulose, carboxymethylcellulose sodium, xanthan gum, carrageenan, calcium sulfate, trisodium phosphate, citric acid and sodium phosphate as buffers, dimethicone antifoam emulsion and preserved with methylparaben and potassium sorbate.

ORA-Plus®, commerically available from Perrigo, comprises purified water, microcrystalline cellulose, carboxymethylcellulose sodium, xanthan gum, carrageenan, calcium sulfate, trisodium phosphate, citric acid and sodium phosphate as buffers, dimethicone antifoam emulsion and preserved with methylparaben and potassium sorbate.

### Results

Experimental results revealed an incompatibility of Compound 1 2HCl with the Orasweet® Sugar Free formulations due to the excipient xanthan gum. Product formed an almost protein-like matrix that wraps around the stir bar and extracted the dye (data not shown). Solubility testing results for Orasweet® Sugar Free formulation and ingredient solubility testing are shown in Tables 38 and 39 respectively. This observation only occurred in Orasweet® Sugar Free options, possibly from xanthan gum. Syrspend® Sugar Free (SF) formulation does not contain xanthan gum and was used for the final vehicle for the stability studies and clinical formulation.

This Example showed that ORA-Sweet® Sugar Free is likely incompatible with Compound 1 2HCl, possibly due to the excipient xanthan gum.

TABLE 38
Solubility Testing Results - Sugar Free.

| | | | API in |
|---|---|---|---|
| API in Flavor | API in 50% Flavor | API in | 50% Versa |
| Sweet | Sweet SF/H₂O | Versa Free | Free/H₂O |
| Precipitate | Precipitate | Precipitate | Precipitate |
| at <5 mg/mL | at <5 mg/mL | at <5 mg/mL | at <5 mg/mL |

TABLE 39
Ingredient Solubility Testing.

| Glycerin in 50% Water | Glycerin |
|---|---|
| >10 mg/mL | >6 mg/mL |

### Example 8

This Example examined the effect of jet milling on particle size distribution of batches of Compound 2 2HCl. In particular, a 51 mm collection loop and a 146 mm collection loop were evaluated.

### Materials and Methods

### Particle Size Distribution (PSD)

Compound 2 API ‘as-received’ (Lots #2064-118-8, #2064-146-9, and # BPR-WS 1828-194D(2HCl)—B1-19) were analyzed for PSD on a Cilas 1180 particle size analyzer. Subsequently jet milled API batches B # L0441-20-JM51mmP1, B # L0441-20-JM51mmP2, B # L0441-20-JM51mmP3, and B # L0441-84-JM146mmP1 were also analyzed for PSD Approximately 50 mg Compound 2-2HCl was dispersed into 40 mL 0.2% (w/w) span 80 in n-hexanes (dispersant) and allowed to mix for 60 minutes. API was kept suspended in dispersant via stirring and sonication during test.

### Jet Milling Studies

A jet milling study was performed on a batch of Compound 2 2HCl with jet mill Fluid Energy Asset #00170 outfitted with a 51 mm collection loop. Batches B # L0441-29-JM51mmP1, B # L0441-29-JM51mmP2, and B # L0441-29-JM51mmP3 were created from ˜10 g of Compound 2 lot # BPR-WS1828-194D(2HCl)-B1-19 subjected to 3 passes. Jet mill settings for grinder nozzle and pusher nozzle as follows: Pass 1 grinder nozzle=60 psi & pusher nozzle=80 psi, Pass 2 and 3 grinder nozzle=50 psi & pusher nozzle=70 psi.

After successfully jet milling on the R&D scale, B # L0441-84-JM146mmP1 was created from Compound 2-2HCl lot # BPR-17-87-B1-21d which was processed with a single pass to confirm GMP scale up conditions in the R&D laboratory by passing 85 g through the GMP jet mill Jet-O-Mizer Asset #01 16 Model 0101 outfitted with 146 mm collection loop using a standard nylon 4×48-inch collection sock inside a PTFE 4×48-inch sock to minimize fines loss. The pressure settings for the grinder and pusher nozzle were: Grinder nozzle 60 psi, Pusher nozzle 70 psi.

### Results

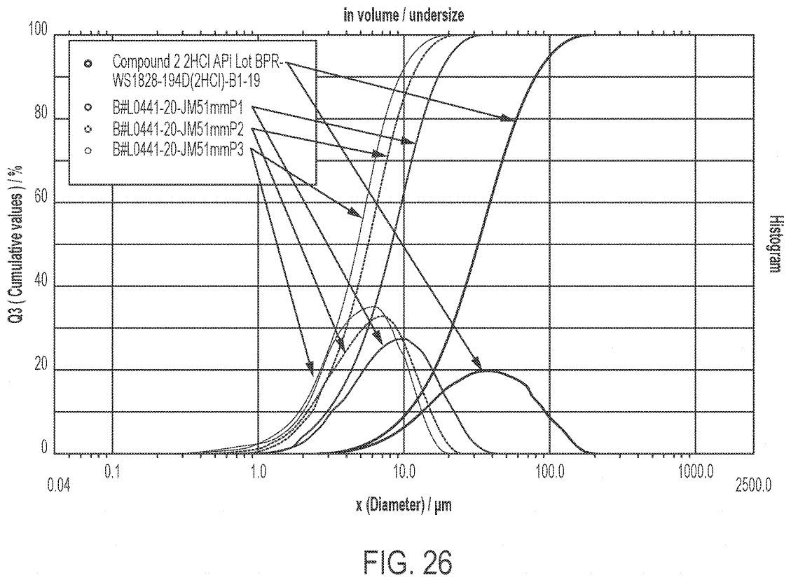

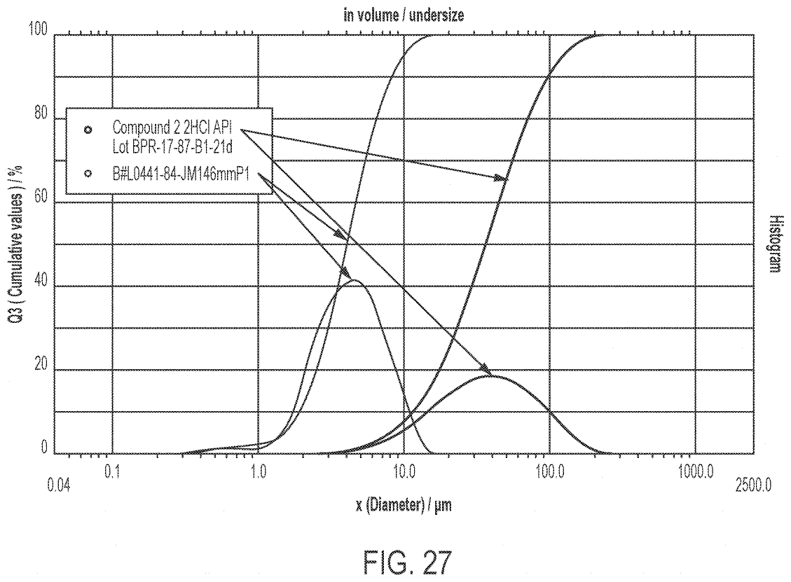

B #132-L0441-20-(12 mg/mL) Triturated was shown to fall out of suspension after 6 days on stability. This was determined to be due to PSD. Two jet milling studies were conducted: (1) R&D Jet Mill outfitted with a 51 mm collection loop, (2) GMP Jet Mill outfitted with 146 mm collection loop. As shown in FIGS. 26-27 and Table 40, jet milling effectively modulated the particle size distribution of Compound 2 2HCl. Table 40 includes the PSD for batches of Compound 2-2HCl API as received (Lot #2064-118-8, 2064-146-9, BPR-WS1828-194D(2HCL)-B1-19, and BPR-17-87-B1-21d) and after jet milling of indicated lots.

TABLE 40
Particle Size Distribution Compound 2-2HCl.

| | | | | d10 | d50 | d90 |
|---|---|---|---|---|---|---|
| API | Lot | Description | n | (μm) | (μm) | (μm) |
| Com- | 2064-118-8 | API as | 2 | 12.0 | 42.8 | 131.6 |
| pound | | received | | | | |
| 2-2HCl | 2064-146-9 | API as | 2 | 8.4 | 23.0 | 57.7 |
| | | received | | | | |
| | BPR-WS1828- | API as | 2 | 11.0 | 33.1 | 83.0 |
| | 194D(2HCL)-B1-19 | received | | | | |
| | BPR-WS1828- | B#L0441-20- | 3 | 3.1 | 7.9 | 17.3 |
| | 194D(2HCL)-B1-19 | JM51mmP1 | | | | |
| | BPR-WS1828- | B#L0441-20- | 3 | 2.3 | 5.6 | 11.7 |
| | 194D(2HCL)-B1-19 | JM51mmP2 | | | | |
| | BPR-WS1828- | B#L0441-20- | 3 | 2.0 | 4.8 | 10.1 |
| | 194D(2HCL)-B1-19 | JM51mmP3 | | | | |
| | BPR-17-87-B1-21d | API as | 3 | 11.6 | 35.7 | 98.3 |
| | | received | | | | |
| | BPR-17-87-B1-21d | B#L0441-84- | 3 | 1.9 | 3.9 | 8.0 |
| | | JM146mmP1 | | | | |

Batches B #132-L0441-20-JM51mmP1, B #132-L0441-20-JM51mmP2, and B #132-L0441-20-JM51mmP3 were created with Compound 2-2HCl API Lot (BPR-WS 1828-194D(2HCl)-B 1-19) and were passed though the jet mill in 3 passes. Table 41 lists the amounts jet milled and their losses for each pass. The small collection loop and back-pressure issues resulted in higher % loss of API. Jet mill passes are described in detail below.

TABLE 41
Jet Mill 51 mm Collector Loop Results.
Jet-Mill 51 mm Loop Compound 2-2HCl
Lot#BPR-WS-1828-194D(2HCl)-B1-19

| | | Start | Collected | Loss | % |
|---|---|---|---|---|---|
| Process | Batch# | (g) | (g) | (g) | Loss |
| Jet Mill 51 mm | B#L0441-20- | 10.0 | 8.155 | 1.845 | 18.5 |
| Loop Pass 1 | JM51mmP1 | | | | |
| Jet Mill 51 mm | B#L0441-20- | 6.155 | 1.68 | 4.475 | 72.7 |
| Loop Pass 2 (a) | JM51mmP2* | | | | |
| Jet Mill 51 mm | B#L0441-20- | 5.0 | 4.44 | 0.56 | 11.2 |
| Loop Pass 2 (b) | JM51mmP2* | | | | |
| Jet Mill 51 mm | B#L0441-20- | 4.12 | 2.53 | 1.59 | 38.6 |
| Loop Pass 3 | JM51mmP3 | | | | |

*Pass a&b combined into one batch.

### Jet Mill (51 mm Collector Loop) Pass 1 B #132-L0441-20-JM51mmP1

Jet Mill Pass 1 created batch B #132-L0441-20-JM51mmP1. Initially 10 g Compound 2-2HCl was jet milled and 8.155 g collected after the first pass. 2.0 grams of pass 1 was retained for testing. Pass 1 had a loss of 18.5%. Settings: pusher jet 80 psi, grinder jet 70 psi.

The first jet mill pass produced the greatest reduction in particle size achieving a d10, d50, d90 (3.1, 7.9, 17.3 μm) with the span of 14.2 μm.

### Jet Mill (51 mm Collector Loop) Pass 2 B #132-L0441-20-JM51mmP2

Jet Mill Pass 2 created batch B #132-L0441-20-JM51mmP2. The second pass 2(A) started with 6.155 g Compound 2-2HCl and encountered severe backpressure, resulting in a loss of 4.475 g with 1.68 g collected. The pusher and grinder jet pressures were changed to 70 and 50 psi respectively to prevent clogging. Due to insufficient material to retain for testing 5.0 g of initial Compound 2-2HCl API Lot (BPR-WS 1828-194D(2HCl)-B 1-19) was passed through the system 2(B) two times which collected 4.44 g using the new settings. The collected Compound 2-2HCl of jet mill passes 2A and 2B were combined (6.12 g). 2.0 grams of combined runs 2A and 2B was retained for testing. Run 2(A) had a loss of 72.7%, but after correcting the back-pressure issue Run 2(B) had a total loss after two passes of 11.2%.

The second jet mill pass modestly reduced particle size further achieving a d10 d50 d90 (2.3, 5.6, 11.7 μm) with the span of 9.4 μm. The second pass tightened the PSD distribution.

Jet Mill (51 mm Collector Loop) Pass 3 B #132-L0441-20-JM51 mmP3

Jet Mill Pass 3 created batch B #132-L0441-20-JM51mmP3. 4.12 g Compound 2-2HCl was jet milled and 2.53 grams was collected for a loss of 38.6%.

The third jet mill pass slightly reduced particle size and span resulting with a d10 d50 d90 (2.0, 4.8, 10.1 μm) with the span of 8.1 μm. The third pass did not significantly change PSD distribution nor PSD span.

### GMP Jet Mill Study (146 mm Collector Loop)

Batch B #132-L0441-84-JM146mmP1 was created with Compound 2-2HCl API Lot BPR-17-87-B 1-21d by a single jet mill pass. 85 g Compound 2-2HCl was passed through a Jet-Mill for a single pass over two days. The overall % loss was 14.1% (73 g obtained from 85 g). Table 42 lists the amounts jet milled and losses for each pass.

TABLE 42
GMP Jet Mill 146 mm Collector Loop Results.
Jet-Mill GMP 146 mm Loop Compound 2-2HCl
Lot#BPR-17-87-B1-21d (Scale Up Test)

| | | Start | Collected | Loss | % |
|---|---|---|---|---|---|
| Process | Batch# | (g) | (g) | (g) | Loss |
| Jet Mill 146 mm | B#L0441-84- | 37.0 | 27 | 10.0 | 27.0 |
| Loop Pass 1 Day 1 | JM146mmP1* | | | | |
| Jet Mill 146 mm | B#L0441-84- | 48.0 | 46 | 2.0 | 4.2 |
| Loop Pass 1 Day 2 | JM146mmP1* | | | | |
| | Total | 85.0 | 73.0 | 12.0 | 14.1 |

*(Pass 1 from Day 1 & 2 combined into one batch)

### GMP Jet Mill Results Day 1 (146 mm Collector Loop)

Day 1 resulted in high losses after single pass through GMP Jet Mill at scale in the R&D laboratory. Day one milled 37 g Compound 2-2HCl with a recovery of 27 g (27% loss). The collection sock used was a standard collection sock. The situation was evaluated revealing the larger collection loop 146 mm produced smaller particles than anticipated <2 μm fines that resulted in higher losses on day one of the single jet mill pass. A change to the collection sock was implemented. The change incorporated the use of a second PTFE lined sock which covered the primary standard collection sock. All other parameters were kept the same.

### GMP Jet Mill Results Day 2 (146 mm Collector Loop)

Day 2 resulted in low losses after a single pass. Day 2 milled 48 g Compound 2-2HCl with a recovery of 46 g (4.2% loss). The incorporation of a second PTFE lined collection sock covering the primary standard collection sock stopped the losses seen previously.

FIG. 27 and Table 40 show the PSD distribution results for the GMP jet mill study.

This Example demonstrates that the particle size distribution for batches of Compound 2-2HCl can be modified using jet milling.

### Example 9

### 7-Day Suspendability-Stability Study of Compound 2-2HCl in Syrspend® SF Cherry

This study evaluated stability & suspendability of Compound 2-2HCl in Syrspend® SF at (12 mg/mL) using 2 jet milled batches of Compound 2-2HCl B # L0441-20-JM51mmP1 (d90 17 um) and B # L0441-20-JM51mmP2 (d90 11 um). The study was conducted for seven days, with samples stored at 25° C. and 40° C./75% RH.

### Materials and Methods

Four batches of 12 mg/mL Compound 2-2HCl/Syrspend® SF Cherry were prepared with two different d90 particle sizes (11 and 17 μm). Samples were tested over 7 days at two stress conditions 25° C. and 40° C./75% RH. Appearance was taken with care as not to disturb the sample on test. HPLC analysis was performed on T=0 and T=7D samples. At T=7D the samples were prepped twice: (1) Settled and (2) Mixed to ascertain suspendability of Compound 2-2HCl in Syrspend® SF Cherry.

### Results

All samples exhibited as a homogenous white/off white suspension for the duration of the test, no indication of Compound 2-2HCl falling out of suspension was observed.

Table 43 lists the % Assay for each timepoint tested. All formulations maintained Compound 2-2HCl in suspension. Two discrepancies occurred with a root cause related to air bubbles remaining during analytical prep transfer resulting from the use of a positive displacement pipette. The first discrepancy was observed in sample B #132-18003-17-(12 mg/mL)-25° C. T=7D settled, where 89.7% Assay was reported. This is not connected with settling as the B #132-18001-17-(12 mg/mL)- at a greater stress level 40° C./75% RH T=7D settled sample had % Assay of 97.8%. The second discrepancy occurred with B #132-18004-11-(12 mg/mL) 40° C./75% RH T=7D mixed. This sample reported a % Assay value of 78.4%. Air bubbles were observed in the quantitative transfer during sample prep due to vigorous mixing. The settled sample prepared prior to agitation (B #132-18004-11-(12 mg/mL)-40° C./75% RH) had an % Assay of 102.2%.

TABLE 43
HPLC Analysis Results.

| | | | T = 7 D | T = 7 D |
|---|---|---|---|---|
| | | T = 0 | Settled | Mixed |
| Sample | Condition | % Assay | % Assay | % Assay |
| B#132-18001-17- | 25° C. | 99.1 | 89.7 | 101.1 |
| (12 mg/mL) | | | | |
| B#132-18001-17- | 40° C./75% RH | 102.9 | 97.8 | 99.6 |
| (12 mg/mL) | | | | |
| B#132-18002-11- | 25° C. | 101 | 96.9 | 97.8 |
| (12 mg/mL) | | | | |
| B#132-18004-11- | 40° C./75% RH | 100.8 | 102.2 | 78.4 |
| (12 mg/mL) | | | | |

This Example demonstrates that jet milling can be used to reduce particle size of batches of Compound 2-2HCl and improve suspendability of Compound 2-2HCl in SyrSpend® SF solution. Jet milled Compound 2 2HCl was also stable.

### ASPECTS AND EMBODIMENTS OF THE INVENTION

Aspects and embodiments of the invention include the subject matter of the following clauses:

Clause 1. A minitablet comprising
an Hsp90 inhibitor,
a binder/diluent, optionally microcrystalline cellulose,
a disintegrant, optionally crospovidone,
an anti-tack agent/flow aid, optionally colloidal silicon dioxide, and
a lubricant, optionally magnesium stearate,
   optionally wherein the minitablet is a delayed release minitablet further comprising

a delayed release coating comprising
a delayed release polymer, optionally methacrylic acid copolymer
a plasticizer, optionally triethyl citrate, and
anti-tack agent/flow aids, optionally colloidal silicon dioxide and/or talc, optionally wherein the delayed release minitablet is a slow release, medium release or fast release minitablet.

Clause 2. A delayed release capsule (or capsular formulation) comprising

one or more minitablets, each comprising
an Hsp90 inhibitor,
a binder/diluent, optionally microcrystalline cellulose,
a disintegrant, optionally crospovidone,
an anti-tack agent/flow aid, optionally colloidal silicon dioxide, and
a lubricant, optionally magnesium stearate, and

a delayed release coating comprising
a delayed release polymer, optionally methacrylic acid copolymer
a plasticizer, optionally triethyl citrate,
anti-tack agent/flow aids, optionally colloidal silicon dioxide and/or talc, and

a capsule, optionally an HMPC capsule.

Clause 3. The delayed release capsule (or capsular formulation) of clause 2, comprising as a w/w percentage of the total weight of the capsule,

in the minitablet,
about 70-80% Hsp90 inhibitor,
about 3-4% binder/diluent, optionally microcrystalline cellulose,
about 4-5% disintegrant, optionally crospovidone,
about 1-2% anti-tack agent/flow aid, optionally colloidal silicon dioxide, and
about 0.1-2% lubricant, optionally magnesium stearate, and

in the delayed release coating,
about 8-9% delayed release polymer, optionally methacrylic acid copolymer
about 1-2% plasticizer, optionally triethyl citrate,
about 1-2% anti-tack agent/flow aid, optionally colloidal silicon dioxide and/or talc.

Clause 4. The delayed release capsule (or capsular formulation) of clause 2 or 3, comprising one or more minitablets.

Clause 5. A minitablet comprising
an Hsp90 inhibitor,
a binder/diluent, optionally microcrystalline cellulose,
a disintegrant, optionally crospovidone,
an anti-tack agent/flow aid, optionally colloidal silicon dioxide, and
a lubricant, optionally magnesium stearate,
   optionally wherein the minitablet is an extended release minitablet and further comprises

a delayed release coating comprising
a delayed release polymer, optionally methacrylic acid copolymer
a plasticizer, optionally triethyl citrate,
anti-tack agent/flow aids, optionally colloidal silicon dioxide and/or talc, and

an extended release coating comprising
a plasticizer, optionally triethyl citrate,
anti-tack agent/flow aids, optionally colloidal silicon dioxide and/or talc, and
a rate controlling polymer, optionally ammonio methacrylate copolymer.

Clause 6. An extended release capsule (or capsular formulation) comprising

a minitablet comprising
an Hsp90 inhibitor,
a binder/diluent, optionally microcrystalline cellulose,
a disintegrant, optionally crospovidone,
an anti-tack agent/flow aid, optionally colloidal silicon dioxide, and
a lubricant, optionally magnesium stearate,

a delayed release coating comprising
a delayed release polymer, optionally methacrylic acid copolymer
a plasticizer, optionally triethyl citrate,
anti-tack agent/flow aids, optionally colloidal silicon dioxide and/or talc,

an extended release coating comprising
a plasticizer, optionally triethyl citrate,
anti-tack agent/flow aids, optionally colloidal silicon dioxide and/or talc, and
a rate controlling polymer, optionally ammonio methacrylate copolymer, and

a capsule, optionally an HMPC capsule.

Clause 7. The extended release capsule (or capsular formulation) of clause 6, comprising as a w/w percentage of the total weight of the capsule

in the minitablet,
about 70-80% Hsp90 inhibitor,
about 3-4% binder/diluent, optionally microcrystalline cellulose,
about 4-5% disintegrant, optionally crospovidone,
about 1-2% anti-tack agent/flow aid, optionally colloidal silicon dioxide, and
about 0.1-2% lubricant, optionally magnesium stearate,

in the delayed release coating,
about 7-10% delayed release polymer, optionally methacrylic acid copolymer
about 1-2% plasticizer, optionally triethyl citrate,
about 2-4% anti-tack agent/flow aids, optionally colloidal silicon dioxide and/or talc,

in the extended release coating,
about 0.5-2% plasticizer, optionally triethyl citrate,
about 0.1-1.5% anti-tack agent/flow aids, optionally colloidal silicon dioxide and/or talc, and
about 0.01-1% rate controlling polymer, optionally ammonio methacrylate copolymer.

Clause 8. The extended release capsule (or capsular formulation) of clause 6 or 7, wherein the capsule is a slow release, medium release or fast release capsule.

Clause 9. A capsule (or capsular formulation) comprising

an Hsp90 inhibitor,

a diluent, optionally microcrystalline cellulose,

a disintegrant, optionally croscarmellose sodium,

a lubricant, optionally magnesium stearate, and

a capsule, optionally a gelatin capsule.

Clause 10. The capsule (or capsular formulation) of clause 9, comprising as a w/w percentage of the total weight of the capsule

about 20-30% Hsp90 inhibitor,

about 70-80% diluent, optionally microcrystalline cellulose,

about 0.1-1% disintegrant, optionally croscarmellose sodium,

about 0.1-1% lubricant, optionally magnesium stearate, and

a capsule, optionally a gelatin capsule.

Clause 11. A capsule (or capsular formulation) comprising

an Hsp90 inhibitor,

povidone or povidone derivative, methacrylic acid copolymer, amino methacrylate copolymer hypromellose acetate succinate or hypromellose,

microcrystalline cellulose,

croscarmellose sodium,

magnesium stearate, and

a capsule,

optionally wherein components of the capsule are prepared using hot melt extrusion.

Clause 12. The capsule (or capsular formulation) of clause 11, comprising as a w/w percentage of the total weight of the capsule

about 5-15% Hsp90 inhibitor,

about 20-30% povidone, or povidone derivative, methacrylic acid copolymer, amino methacrylate copolymer hypromellose acetate succinate or hypromellose,

about 50-65% microcrystalline cellulose,

about 5-15% croscarmellose sodium, and

about 0.5-1.5% magnesium stearate.

Clause 13. A capsule (or capsular formulation) comprising

a Hsp90 inhibitor,

a binder, optionally Gelucire 50/13,

a diluent, optionally lactose monohydrate,

a disintegrant, optionally croscarmellose sodium, and

a capsule,

optionally wherein components of the capsule are prepared using hot melt granulation.

Clause 14. The capsule (or capsular formulation) of clause 13, comprising as a w/w percentage of the total weight of the capsule

about 1-44% Hsp90 inhibitor,

about 10-30% binder, optionally Gelucire 50/13,

about 30-73% diluent, optionally lactose monohydrate, and

about 1-10% disintegrant, optionally croscarmellose sodium.

Clause 15. A capsule (or capsular formulation) comprising

an Hsp90 inhibitor, and
a disintegrant, optionally croscarmellose sodium.

Clause 16. A capsule (or capsular formulation) comprising

an Hsp90 inhibitor, and
sodium starch glycolate.

Clause 17. A capsule (or capsular formulation) comprising

a hot melt micronized Hsp90 inhibitor, and
Glycerol Monostearate.

Clause 18. A capsule (or capsular formulation) comprising

a hot melt micronized Hsp90 inhibitor, and
Gelucire.

Clause 19. A capsule (or capsular formulation) comprising

a hot melt micronized Hsp90 inhibitor, and
Vitamin E TPGS.

Clause 20. A capsule (or capsular formulation) comprising

a hot melt Hsp90 inhibitor, and
Glycerol Monostearate.

Clause 21. A capsule (or capsular formulation) comprising

a hot melt Hsp90 inhibitor, and
Gelucire.

Clause 22. A capsule (or capsular formulation) comprising

a hot melt Hsp90 inhibitor, and
Vitamin E TPGS.

Clause 23. A capsule (or capsular formulation) comprising

micronized Hsp90 inhibitor.

Clause 24. A capsule (or capsular formulation) comprising

micronized blend of Hsp90 inhibitor.

Clause 25. A spray dry dispersion tablet comprising an Hsp90 inhibitor and one or more excipients as provided in Table 10, and wherein the PVP VA can be substituted with HPMC AS or PVP K30, and wherein Compound 1 can be substituted with another Hsp90 inhibitor such as but not limited to Compound 1a, Compound 2, and Compound 2a.

Clause 26. The spray dry dispersion tablet of clause 25, wherein the ratio of PVP VA to Compound 1, as provided in Table 10, can be substituted with 1:1 or 2:1.

Clause 27. A tablet comprising
an Hsp90 inhibitor
one or more fillers/bulking agents, optionally lactose, microcrystalline cellulose, mannitol, and/or povidone,
one or more disintegrants, optionally hydroxypropyl cellulose and/or croscarmellose sodium,
an eluant, optionally fumed silica, and
one or more lubricants, optionally magnesium stearate and/or sodium stearyl fumarate,
optionally wherein the tablet is prepared using a wet granulation-dry blend (WG-DB) method.

Clause 28. The tablet of clause 27, further comprising an immediate release coating.

Clause 29. The tablet of clause 27, further comprising a delayed release coating.

Clause 30. A capsule (or capsular formulation) comprising

an Hsp90 inhibitor,

cornstarch,

microcrystalline cellulose,

fumed silicon dioxide,

polysorbate 80

gelatin,

water,

magnesium stearate, and

a capsule,

optionally wherein components of the capsule are prepared using wet granulation.

Clause 31. An oral disintegrating tablet comprising

an Hsp90 inhibitor,

a filler or binder, optionally mannitol (e.g., Pearlitol 300DC), sucrose, silicified microcrystalline cellulose (e.g., prosolv HD90), or lactose,

a disintegrant, optionally crospovidone (e.g., polyplasdone XL), L-HPC, Pharmaburst, PanExcea, or F-Melt,

a lubricant, optionally Pruv or Lubripharm, and/or

a glidant, optionally fumed silica, and/or

a dispersion agent, optionally calcium silicate.

Clause 32. The minitablet, capsule (or capsular formulation) or tablet of any one of the foregoing clauses, wherein the Hsp90 inhibitor has a structure of any one of Formulae I-XIV.

Clause 33. The minitablet, capsule (or capsular formulation) or tablet of any one of the foregoing clauses, wherein the Hsp90 inhibitor is Compound 1 or Compound 1a, optionally in a salt form, further optionally in a dihydrochloride form.

Clause 34. The minitablet, capsule (or capsular formulation) or tablet of any one of the foregoing clauses, wherein the Hsp90 inhibitor is Compound 2 or Compound 2a, optionally in a free base form or a salt form, further optionally wherein the salt form is a dihydrochloride form.

Clause 35. The minitablet, capsule (or capsular formulation) or tablet of any one of the following clauses, comprising a dosage strength of at least 0.1 mg, at least 0.5 mg, at least 1 mg, at least 5 mg, at least 10 mg, at least 50 mg, or at least 100 mg of the Hsp90 inhibitor, or a 0.1 mg, 0.5 mg, 1 mg, 5 mg, 10 mg, 50 mg, or 100 mg dosage strength of the Hsp90 inhibitor.

Clause 36. The minitablet, capsule (or capsular formulation) or tablet of any one of the following clauses, provided as a plurality in a container.

Clause 37. The minitablet, capsule (or capsular formulation) or tablet of any one of the following clauses, provided in a container with a dessicant.

Clause 38. An orally administered solution comprising an Hsp90 inhibitor.

Clause 39. An orally administered suspension comprising an Hsp90 inhibitor.

Clause 40. The orally administered solution or suspension of clause 38 or 39, wherein the Hsp90 inhibitor has a structure of any one of Formulae I-XIV, and may be in a salt or free base form.

Clause 41. The orally administered solution or suspension of clause 38 or 39, wherein the Hsp90 inhibitor is Compound 1 or Compound 1a, optionally in a salt form, further optionally in a dihydrochloride form.

Clause 42. The orally administered solution or suspension of clause 38 or 39, wherein the Hsp90 inhibitor is Compound 2 or Compound 2a, optionally in a free base form or a salt form, further optionally wherein the salt form is a dihydrochloride form.

Clause 43. The orally administered solution or suspension of any one of clauses 38-42, comprising a dosage strength of at least 0.1 mg, at least 0.5 mg, at least 1 mg, at least 5 mg, at least 10 mg, at least 50 mg, or at least 100 mg of the Hsp90 inhibitor, or a 0.1 mg, 0.5 mg, 1 mg, 5 mg, 10 mg, 50 mg, or 100 mg dosage strength of the Hsp90 inhibitor.

Clause 44. The orally administered solution or suspension of any one of clauses 38-43, further comprising methylcellulose.

Clause 45. The orally administered solution or suspension of any one of clauses 38-43, further comprising Captisol®.

Clause 46. The orally administered solution or suspension of any one of clauses 38-43, further comprising water, modified food starch(es), sodium citrate, sucralose, buffer(s), anti-foaming agent(s), and preservatives(s), optionally wherein the buffer(s) are citric acid, sorbic acid, and malic acid and/or optionally wherein the anti-foaming agent(s) is simethicone and/or optionally wherein the preservative(s) is sodium benzoate (e.g., <0.1% sodium benzoate).

Clause 47. The orally administered solution or suspension of any one of clauses 38-46, further comprising buffer(s) and preservative(s).

Clause 48. The orally administered solution or suspension of any one of clauses 38-47, free of xanthan gum.

Clause 49. A method for treating a subject having a condition characterized by abnormal Hsp90 activity, presence of mis-folded proteins, or responsiveness to Hsp90 inhibition, comprising

administering one or more capsules or tablets or orally administered solutions or suspensions of any one of the foregoing clauses in an effective amount.

Clause 50. The method of clause 49, wherein the condition is a cancer, optionally pancreatic or breast cancer, melanoma, B cell lymphoma, Hodgkin's lymphoma, or non-Hodgkin's lymphoma.

Clause 51. The method of clause 49, wherein the condition is a myeloproliferative neoplasm, optionally myelofibrosis, polycythemia vera (PV) or essential thrombrocythemia (ET).

Clause 52. The method of clause 49, wherein the condition is a neurodegenerative disorder, optionally chronic traumatic encephalopathy, acute traumatic brain injury, ALS, Alzheimer's disease, or Parkinson disease.

Clause 53. The method of clause 49, wherein the condition is an inflammatory condition, optionally a cardiovascular disease such as atherosclerosis, or an autoimmune disease.

Clause 54. The method of any one of clauses 49-53, further comprising administering a secondary therapeutic agent to the subject.

Clause 55. The method of any one of clauses 49-54, wherein the capsules or tablets or orally administered solutions or suspensions are administered daily, every 2 days, every 3 days, every 4 days, every 5 days, every 6 days, every week, every 2 weeks, every 3 weeks, every 4 weeks, every month, every 2 months, every 3 months, every 4 months, every 6 months, or every year, optionally with a non-treatment period between any two consecutive treatment periods.

Clause 56. The method of any one of clauses 49-54, wherein the capsules or tablets or orally administered solutions or suspensions are administered once a day, twice a day, or thrice a day.

Clause 57. The method of any one of clauses 49-54, wherein the capsules or tablets or orally administered solutions or suspensions are administered every 3 hours, every 4 hours, every 6 hours, every 12 hours, or every 24 hours.

Clause 58. A method for treating a subject having a condition characterized by abnormal Hsp90 activity, presence of mis-folded proteins, or responsiveness to Hsp90 inhibition, comprising

administering one or more capsules or tablets or orally administered solutions or suspensions comprising one or more Hsp90 inhibitors of any one of Formulae I-XIV and one or more secondary therapeutic agents in a therapeutically effective amount.

Clause 59. The method of clause 58, wherein the one or more Hsp90 inhibitors are co-administered with the one or more secondary therapeutic agents.

Clause 60. The method of any one of clauses 49-59, wherein the capsules or tablets or orally administered solutions or suspensions comprise Compound 1, Compound 1a, Compound 2 or Compound 2a, in free base or salt form.

Clause 61. The method of clause 60, wherein the salt form is a dihydrochloride form.

### OTHER EMBODIMENTS AND EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A minitablet comprising
an Hsp90 inhibitor,
a binder/diluent, optionally microcrystalline cellulose,
a disintegrant, optionally crospovidone,
an anti-tack agent/flow aid, optionally colloidal silicon dioxide, and
a lubricant, optionally magnesium stearate,
optionally wherein the minitablet is a delayed release minitablet and further comprises a delayed release coating comprising
a delayed release polymer, optionally methacrylic acid copolymer
a plasticizer, optionally triethyl citrate, and
anti-tack agent/flow aids, optionally colloidal silicon dioxide and/or talc.

2. A delayed release capsule formulation comprising
a minitablet comprising
an Hsp90 inhibitor,
a binder/diluent, optionally microcrystalline cellulose,
a disintegrant, optionally crospovidone,
an anti-tack agent/flow aid, optionally colloidal silicon dioxide, and
a lubricant, optionally magnesium stearate, and
a delayed release coating comprising
a delayed release polymer, optionally methacrylic acid copolymer
a plasticizer, optionally triethyl citrate,
anti-tack agent/flow aids, optionally colloidal silicon dioxide and/or talc, and
a capsule, optionally an HMPC capsule.

3. A minitablet comprising
an Hsp90 inhibitor,
a binder/diluent, optionally microcrystalline cellulose,
a disintegrant, optionally crospovidone,
an anti-tack agent/flow aid, optionally colloidal silicon dioxide, and
a lubricant, optionally magnesium stearate,
optionally wherein the minitablet is an extended release minitablet and further comprises
a delayed release coating comprising
a delayed release polymer, optionally methacrylic acid copolymer
a plasticizer, optionally triethyl citrate,
anti-tack agent/flow aids, optionally colloidal silicon dioxide and/or talc, and
an extended release coating comprising
a plasticizer, optionally triethyl citrate,
anti-tack agent/flow aids, optionally colloidal silicon dioxide and/or talc, and
a rate controlling polymer, optionally ammonio methacrylate copolymer.

4. An extended release capsule formulation comprising
a minitablet comprising
an Hsp90 inhibitor,
a binder/diluent, optionally microcrystalline cellulose,
a disintegrant, optionally crospovidone,
an anti-tack agent/flow aid, optionally colloidal silicon dioxide, and
a lubricant, optionally magnesium stearate,
a delayed release coating comprising
a delayed release polymer, optionally methacrylic acid copolymer
a plasticizer, optionally triethyl citrate,
anti-tack agent/flow aids, optionally colloidal silicon dioxide and/or talc,
an extended release coating comprising
a plasticizer, optionally triethyl citrate,
anti-tack agent/flow aids, optionally colloidal silicon dioxide and/or talc, and
a rate controlling polymer, optionally ammonio methacrylate copolymer, and
a capsule, optionally an HMPC capsule.

5. An capsule formulation comprising
an Hsp90 inhibitor,
a diluent, optionally microcrystalline cellulose,
a disintegrant, optionally croscarmellose sodium,
a lubricant, optionally magnesium stearate, and
a capsule, optionally a gelatin capsule.

6. A capsule formulation comprising
an Hsp90 inhibitor,
povidone or povidone derivative, methacrylic acid copolymer, amino methacrylate copolymer hypromellose acetate succinate or hypromellose,
microcrystalline cellulose,
croscarmellose sodium,
magnesium stearate, and
a capsule,
optionally wherein components of the capsule are prepared using hot melt extrusion.

7. A capsule formulation comprising
a Hsp90 inhibitor,
a binder, optionally Gelucire 50/13,
a diluent, optionally lactose monohydrate,
a disintegrant, optionally croscarmellose sodium, and
a capsule,
optionally wherein components of the capsule are prepared using hot melt granulation.

8. A capsule formulation comprising
an Hsp90 inhibitor, and
(a) a disintegrant, optionally croscarmellose sodium, or
(b) sodium starch glycolate.

9. A capsule formulation comprising
a hot melt Hsp90 inhibitor, and
(a) Glycerol Monostearate, or
(b) Gelucire, or
(c) Vitamin E TPGS,
optionally wherein the hot melt Hsp90 inhibitor is a hot melt micronized Hsp90 inhibitor

10. A capsule formulation comprising
(a) micronized Hsp90 inhibitor or
(b) micronized blend of Hsp90 inhibitor.

11. A spray dry dispersion tablet comprising an Hsp90 inhibitor and one or more excipients as provided in Table 10, and wherein the PVP VA can be substituted with HPMC AS or PVP K30, and wherein Compound 1 can be substituted with another Hsp90 inhibitor.

12. A tablet comprising
an Hsp90 inhibitor,
one or more fillers/bulking agents, optionally lactose, microcrystalline cellulose, mannitol, and/or povidone,
one or more disintegrants, optionally hydroxypropyl cellulose and/or croscarmellose sodium,
an eluant, optionally fumed silica, and
one or more lubricants, optionally magnesium stearate and/or sodium stearyl fumarate,
optionally wherein the tablet is prepared using a wet granulation-dry blend (WG-DB) method.

13. A capsule formulation comprising
an Hsp90 inhibitor,
cornstarch,
microcrystalline cellulose,
fumed silicon dioxide,
polysorbate 80
gelatin,
water,
magnesium stearate, and
a capsule,
optionally wherein components of the capsule are prepared using wet granulation.

14. An oral disintegrating tablet comprising
an Hsp90 inhibitor,
a filler or binder, optionally mannitol (e.g., Pearlitol 300DC), sucrose, silicified microcrystalline cellulose (e.g., prosolv HD90), or lactose,
a disintegrant, optionally crospovidone (e.g., polyplasdone XL), L-HPC, Pharmaburst, PanExcea, or F-Melt,
a lubricant, optionally Pruv or Lubripharm, and/or
a glidant, optionally fumed silica, and/or
a dispersion agent, optionally calcium silicate.

15. The capsule formulation or tablet or minitablet of any one of the foregoing claims, wherein the Hsp90 inhibitor has a structure of any one of Formulae I-XIV.

16. The capsule formulation or tablet or minitablet of any one of the foregoing claims, wherein the Hsp90 inhibitor is Compound 1.

17. The capsule formulation or tablet or minitablet of any one of the foregoing claims, wherein the Hsp90 inhibitor is Compound 2.

18. An orally administered solution or suspension comprising an Hsp90 inhibitor.

19. A method for treating a subject having a condition characterized by abnormal Hsp90 activity, presence of mis-folded proteins, or responsiveness to Hsp90 inhibition, comprising
administering one or more capsule formulations or tablets of any one of the foregoing claims in an effective amount.

20. A method for treating a subject having a condition characterized by abnormal Hsp90 activity, presence of mis-folded proteins, or responsiveness to Hsp90 inhibition, comprising
administering one or more capsule formulations or tablets comprising one or more Hsp90 inhibitors of any one of Formulae I-XIV and one or more secondary therapeutic agents in a therapeutically effective amount.
